(19) United States
(12) Patent Application Publication
(10) Pub. No.: US 2024/0294586 A1
(43) Pub. Date: Sep. 5, 2024

(54) TENASCIN-C AUTOANTIGENIC EPITOPES IN RHEUMATOID ARTHRITIS

(71) Applicant: Benaroya Research Institute at Virginia Mason, Seattle, WA (US)

(72) Inventors: Jane Buckner, Seattle, WA (US); Eddie Arthur James, Monroe, WA (US); Jing Song, Seattle, WA (US)

(21) Appl. No.: 18/251,368

(22) PCT Filed: Nov. 3, 2021
(86) PCT No.: PCT/US2021/057943

§ 371 Date: May 1, 2023

Related U.S. Application Data
(60) Provisional application No. 63/109,768, filed on Nov. 4, 2020.

(51) Int. Cl. C07K 14/47; A61K 39/00; G01N 33/564

(52) U.S. Cl. CPC C07K 14/47; A61K 39/0008; A61K 39/4611; A61K 39/4622; A61K 39/4634; A61K 39/46433; G01N 33/564; A61K 2039/5158; G01N 2440/18; G01N 2800/102

(57) ABSTRACT

Compositions and methods are disclosed that relate to the discovery of citrullinated oligopeptides of 9-14 amino acids or 9-18 amino acids, comprising peptide sequences derived from the polypeptide sequence of human tenascin-C (TNC) containing citrullination-dependent immunological epitopes that, remarkably, are antigen-specifically recognized by both autoantibodies and CD4+ T cells from human subjects having rheumatoid arthritis (RA). The citrullinated TNC (citTNC) oligopeptides are useful for isolating autoantibodies from, and detecting autoantibodies in, biological fluids from subjects having or suspected of having RA. Also described are uses of citTNC oligopeptides to prepare compositions for induction of citTNC autoantigen-specific immunological tolerance, including tolerogens and citTNC-specific Treg cells.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 140160_405WO_SEQUENCE_LISTING.txt. The text file is 41.5 KB, was created on Oct. 31, 2021, and is being submitted electronically via EFS-Web.

### BACKGROUND

### Technical Field

The present disclosure relates generally to immunogenic tenascin-C epitopes targeted by autoreactive T-cells and autoantibodies in patients with rheumatoid arthritis. The disclosed epitopes are recognized by both CD4+ T-cells and B cells and may play a role in amplifying autoimmunity to promote the development and progression of rheumatoid arthritis. More specifically, compositions and methods are described pertaining to the use of citrullinated tenascin-C oligopeptides for the diagnosis and treatment of rheumatoid arthritis.

### Description of the Related Art

Autoimmune disease is characterized by a specific adaptive immune response directed against self-antigens. During the course of an immune response, the normal outcome is clearance of a foreign antigen. When an immune response is directed against self-antigens, however, elimination is improbable, creating a sustained response. This persistent response leads to a chronic condition characterized by inflammation and subsequent tissue injury. The majority of adaptive immune lymphoid cells bearing self-reactive antigen-specific receptors are eliminated during development, however, given the broad specificity of T- and B-cell receptors, escape of low-affinity cells still occurs. Further, the triggers for breakdown of tolerance in the periphery and initiation of autoimmunity are not known, although both genetic and environmental factors may contribute (Theofilopoulos et al., 2017 Nat. Immunol. 18:716). Standard treatment for autoimmune disease has relied on immunosuppressive medications to broadly dampen immune responses. Long-term use of immunosuppressive drugs, however, can render patients susceptible to infections and cancer, as well as create significant toxicities and side effects. More recently, immunotherapy has received increased attention in the treatment of disorders characterized by immune system dysregulation including autoimmune disease, with promising outcomes. These therapies have focused on rebalancing the immune system using regulatory (immunosuppressive) antigen-specific T-cells, antigen-specific helper (immunopotentiating) T-cells, tolerogenic dendritic cells, and monoclonal antibodies targeting cytokines and immune checkpoint molecules.

Rheumatoid arthritis (RA) is a chronic autoimmune disease that primarily affects the synovial lining of joints and is associated with cartilage and bone damage as well as progressive disability. Autoantibodies are a hallmark of RA and contribute to the initiation and maintenance of RA-associated inflammation. Rheumatoid factor (RF) and anti-citrullinated protein antibodies (ACPAs) are the two most prevalent autoantibodies identified in rheumatoid arthritis (RA). RF contributes to the formation of large immune complexes in synovial tissues by binding the Fc region of IgG1 antibodies. ACPAs target autoantigenic polypeptide epitopes that have undergone post-translational modification in which an arginine has been deiminated to citrulline by peptidyl arginine deiminases. To date, a diverse array of citrullinated antigens have been reported including α-enolase, cartilage intermediate-layer protein (CILP), fibrinogen, vimentin, type II collagen and aggrecan (Quirk et al., 2011 FEBS Lett. 585:3681; Kinloch et al., 2005 Arthritis Res. Ther. 7:R1421; Du et al., 2005 Rheumatol. Int. 26:35; Takizawa et al., 2006 Ann. Rheum. Dis. 65:1013; Steendam et al., 2010 Arth. Res. Ther. 12:R132; Yoshida et al., 2006 Mod. Rheumatol. 16:276; von Delwig et al., 2010 Arth. Rheum. 62:143). ACPAs have also been shown to be predictive of developing RA in the at-risk population (Rantapss-Dahlqvist et al., 2003 Arth. Rheum. 48:2741).

Tenascin-C is an extracellular matrix protein of considerable clinical interest due to its association with autoimmune diseases, including ulcerative colitis, Crohn's disease, systemic lupus erythematosus, ankylosing spondylitis and rheumatoid arthritis (Riedl et al., 2001; Audeh et al., 2010; Page et al., 2012; Gupta et al.). In clinical studies of RA patients, tenascin-C protein levels are elevated and correlate with disease activity in serum as well as in the synovium of the knee joint (Cutolo et al., 1992; Salter et al., 1993; Goh et al., 2010). Mechanistic studies in mice have further demonstrated that tenascin-C is required for maintenance of synovial inflammation through interaction with toll-like receptor 4 in an arthritis model (Midwood et al., 2009; Zuliani-Alvarez et al., 2017). Additionally, tenascin-C expression in dendritic cells promotes Th17 cell polarization in an antigen-induced arthritis mouse model (Ruhmann et al, 2012). Collectively, these findings suggest that tenascin-C is an important autoantigen in RA, however, the key immunodominant epitopes targeted by autoantibodies and autoreactive T-cells are yet to be determined.

In addition to autoantibodies, autoreactive CD4+ T-cells play a significant role in RA disease pathogenesis. Infiltrating CD4+ T-cells traffic to the joint as part of the inflammatory process and such cells also exhibit an increased likelihood of expressing T-cell receptors (TCR) that-specifically recognize antigens that are present within joint tissues. Importantly, the high level of somatic mutation in RA autoantibodies is suggestive of ongoing T-cell help (Elliott et al., 2018). Consequently, CD4+ T-cells may serve as an important therapeutic target for ameliorating RA. Despite recognition in the art of autoimmune processes that underlie the disease mechanism in RA, including implication of tenascin-C as an autoantigen, detailed understanding of the immunological specificity of pro-arthritogenic autoantigen recognition remains elusive. Clearly, there remains a need in the art for improved understanding of RA pathogenesis, including characterization of the roles of self-protein citrullination and tenascin-C in the induction of autoreactive T-cell and autoantibody responses, and further including definition with fine specificity of autoantigenic epitope structures that may be immunologically significant in RA. The present disclosure addresses this need by describing compositions and methods including tenascin-C autoantigenic epitope-containing peptides and their use in the detection and treatment of RA, and provides other related advantages.

### BRIEF SUMMARY

According to certain embodiments of the present invention, there is provided an isolated citrullinated oligopeptide of no more than 14, 13, 12, 11, 10, or 9 amino acids, comprising a polypeptide of general formula (I) which comprises at least one arginine residue (R) that is citrullinated ([Cit]):

N—X—C (I)

said citrullinated oligopeptide being capable of eliciting an antigen-specific T cell response to human tenascin-C, wherein [R/Cit] is either arginine or citrullinated arginine and wherein: (a) X comprises an amino acid sequence that is selected from: IS[R/Cit][R/Cit]GDMSS (SEQ ID NO: 1) [Peptide 17A] (TNC 874-882/Cit 876-877), SLIS[R/Cit][R/Cit]GDMSSNPA (SEQ ID NO: 2) [Peptide17] (TNC 872-885/Cit 876-877), GQYEL[R/Cit]VDL[R/Cit]DHGE (SEQ ID NO: 3) Peptide 56] (TNC 2068-2081/Cit 2073, 2077), and YEL[R/Cit]VDL[R/Cit]D (SEQ ID NO: 4) [Peptide 56B] (TNC 2070-2078/Cit 2073, 2077); (b) N is an amino terminus of the oligopeptide and consists of 0, 1, 2, 3, 4, or 5 amino acids that are independently selected from natural amino acids and non-natural amino acids; and (c) C is a carboxy terminus of the oligopeptide and consists of 0, 1, 2, 3, 4, or 5 amino acids that are independently selected from natural amino acids and non-natural amino acids.

In certain further embodiments the polypeptide of general formula (I) comprises at least two arginine residues that are citrullinated, and in certain still further embodiments the amino acid sequence of X in general formula (I) comprises said at least two arginine residues that are citrullinated. In certain other further embodiments binding affinity of said oligopeptide of general formula (I) to a MHC class II molecule is greater than the binding affinity to the MHC class II molecule of a hypocitrullinated polypeptide comprising the oligopeptide of general formula (I) in which said at least one arginine residue is non-citrullinated. In certain embodiments said MHC class II molecule is an HLA-DRB1*04:01 molecule. In certain embodiments binding affinity of said oligopeptide of general formula (I) to a MHC class II molecule is greater than the binding affinity to the MHC class II molecule of a hypocitrullinated polypeptide comprising the oligopeptide of general formula (I) in which said at least two arginine residues in the amino acid sequence of X are non-citrullinated. In certain further embodiments said MHC class II molecule is an HLA-DRB1*04:01 molecule.

In certain embodiments the antigen-specific T cell response is a CD4+ T cell response. In certain further embodiments the CD4+ T cell is a regulatory T (Treg) cell. In certain embodiments there is provided a composition comprising the above described isolated citrullinated oligopeptide and a carrier. In certain embodiments the isolated citrullinated oligopeptide is bound to the carrier noncovalently or covalently. In certain embodiments the carrier is a solid carrier, which in certain further embodiments comprises a nanoparticle, a microparticle, a macroparticle, or a magnetic bead, or the composition comprises an immunoassay substrate in which the solid carrier is a tube, an assay plate, a well of a multi-well plate, a membrane, a nanoparticle, a microparticle, a macroparticle, or a magnetic bead.

Turning to another embodiment there is provided a method of detecting presence in a biological sample of an antibody that specifically binds to citrullinated human tenascin C, comprising: (a) contacting (i) a biological sample obtained from a subject having or suspected of having one or more antibodies that specifically bind to human tenascin C, with (ii) an isolated citrullinated oligopeptide of no more than 14, 13, 12, 11, 10, or 9 amino acids, comprising a polypeptide of general formula (I) which comprises at least one arginine residue (R) that is citrullinated ([Cit]): N—X—C(I) wherein [R/Cit] is either arginine or citrullinated arginine, under conditions and for a time sufficient for specific binding of said one or more antibodies to the citrullinated oligopeptide; and (b) determining a test level of specific binding of said one or more antibodies to the citrullinated oligopeptide that is greater than a control level of specific binding of said one or more antibodies to a non-citrullinated polypeptide comprising the oligopeptide of general formula (I) in which there is no citrullinated arginine residue, and therefrom detecting presence in the biological sample of an antibody that specifically binds to citrullinated human tenascin C, wherein in general formula (I): (1) X comprises an amino acid sequence that is selected from: IS[R/Cit][R/Cit]GDMSS (SEQ ID NO: 1) [Peptide 17A] (TNC 874-882/Cit 876-877), SLIS[R/Cit][R/Cit]GDMSSNPA (SEQ ID NO: 2) [Peptide 17] (TNC 872-885/Cit 876-877), GQYEL[R/Cit]VDL[R/Cit]DHGE (SEQ ID NO: 3) [Peptide 56] (TNC 2068-2081/Cit 2073, 2077), and YEL[R/Cit]VDL[R/Cit]D (SEQ ID NO: 4) [Peptide 56B](TNC 2070-2078/Cit 2073, 2077); (2) N is an amino terminus of the oligopeptide and consists of 0, 1, 2, 3, 4, or 5 amino acids that are independently selected from natural amino acids and non-natural amino acids; and (3) C is a carboxy terminus of the oligopeptide and consists of 0, 1, 2, 3, 4, or 5 amino acids that are independently selected from natural amino acids and non-natural amino acids.

In certain embodiments, in the citrullinated oligopeptide the amino acid sequence of X in general formula (I) comprises two arginine residues that are citrullinated. In certain embodiments the subject has or is suspected of having rheumatoid arthritis. In certain embodiments the biological sample comprises blood, plasma, serum, lymph, synovial fluid, saliva, sputum, stool, or bronchoalveolar lavage (BAL) fluid. In certain embodiments the isolated citrullinated oligopeptide further comprises a carrier. In certain further embodiments the isolated citrullinated oligopeptide is bound to the carrier noncovalently or covalently. In certain embodiments the carrier is a solid carrier. In certain embodiments the isolated citrullinated oligopeptide comprises an immunoassay substrate in which the solid carrier is selected from a tube, an assay plate, a well of a multi-well plate, a membrane, a nanoparticle, a microparticle, a macroparticle, or a magnetic bead.

In certain embodiments there is provided an antigen-specific immunomodulatory composition comprising the above described isolated citrullinated oligopeptide and a pharmaceutically acceptable carrier, wherein the immunomodulatory composition is capable of inducing human tenascin C-specific immunological tolerance following administration to a human subject. In certain embodiments the isolated citrullinated oligopeptide is bound to the carrier noncovalently or covalently. In certain embodiments the carrier is a solid carrier. In certain embodiments the solid carrier comprises a nanoparticle. In certain embodiments the composition induces tenascin C-specific immunological tolerance that is MHC class II molecule-restricted, wherein the MHC class II molecule is HLA-DRB1*04:01.

In certain embodiments there is provided a method of isolating one or a plurality of tenascin C-specific antibodies from a biological sample, comprising: (a) incubating a reaction mixture that is formed by contacting (i) a biological sample obtained from a subject having or suspected of having one or more antibodies that specifically bind to human tenascin C, with (ii) an artificial antigen that comprises an isolated citrullinated oligopeptide of no more than 14, 13, 12, 11, 10, or 9 amino acids, comprising a polypeptide of general formula (I) which comprises at least one arginine residue (R) that is citrullinated ([Cit]): N—X—C(I) wherein [R/Cit] is either arginine or citrullinated arginine, under conditions and for a time sufficient for specific binding of said one or more antibodies from the biological sample to the citrullinated oligopeptide to form one or more immune complexes comprising said antibodies specifically bound to the oligopeptide; and (b) removing from the reaction mixture antibodies from the biological sample that are not specifically bound to the citrullinated oligopeptide, to recover said one or more immune complexes from the reaction mixture, and thereby isolating one or a plurality of tenascin-C-specific antibodies, wherein in general formula (I): (1) X comprises an amino acid sequence that is selected from: IS[R/Cit][R/Cit]GDMSS (SEQ ID NO: 1) [Peptide 17A] (TNC 874-882/Cit 876-877), SLIS[R/Cit][R/Cit]GDMSSNPA (SEQ ID NO: 2) [Peptide 17] (TNC 872-885/Cit 876-877), GQYEL[R/Cit]VDL[R/Cit]DHGE (SEQ ID NO: 3) [Peptide 56] (TNC 2068-2081/Cit 2073, 2077), and YEL[R/Cit]VDL[R/Cit]D (SEQ ID NO: 4) [Peptide 56B](TNC 2070-2078/Cit 2073, 2077); (2) N is an amino terminus of the oligopeptide and consists of 0, 1, 2, 3, 4, or 5 amino acids that are independently selected from natural amino acids and non-natural amino acids; and (3) C is a carboxy terminus of the oligopeptide and consists of 0, 1, 2, 3, 4, or 5 amino acids that are independently selected from natural amino acids and non-natural amino acids.

In certain embodiments the method further comprises, following step (b) of removing, (c) quantifying the one or more immune complexes that have been recovered. In certain embodiments step (c) of quantifying comprises determining a test level of specific binding of said one or more antibodies to the citrullinated oligopeptide relative to a control level of specific binding of said one or more antibodies to a non-citrullinated polypeptide comprising the oligopeptide of general formula (I) in which there is no citrullinated arginine residue. In certain embodiments, in the citrullinated oligopeptide the amino acid sequence of X in general formula (I) comprises two arginine residues that are citrullinated. In certain embodiments the subject has or is suspected of having rheumatoid arthritis. In certain embodiments the biological sample comprises blood, plasma, serum, lymph, synovial fluid, saliva, sputum, stool, or bronchoalveolar lavage (BAL) fluid.

In certain embodiments there is provided a method for in vitro preparation of antigen-pulsed antigen-presenting cells that are immunocompatible with a subject, comprising: contacting in vitro, under conditions and for a time sufficient for antigen processing and presentation by antigen-presenting cells to take place, (i) a population of antigen-presenting cells that are immunocompatible with the subject, and (ii) the above described isolated citrullinated oligopeptide, thereby obtaining antigen-pulsed antigen-presenting cells capable of eliciting an antigen-specific T cell response to human tenascin-C. In certain embodiments the antigen-specific T cell response is a CD4+ T cell response. In certain embodiments the CD4+ T cell is a regulatory T (Treg) cell. In certain further embodiments the Treg cell responds antigen-specifically to a citrullinated oligopeptide which comprises at least one arginine residue (R) that is citrullinated ([Cit]), said citrullinated oligopeptide comprising an amino acid sequence that is selected from: IS[R/Cit][R/Cit]GDMSS (SEQ ID NO: 1) [Peptide 17A] (TNC 874-882/Cit 876-877), SLIS[R/Cit][R/Cit]GDMSSNPA (SEQ ID NO: 2) [Peptide 17] (TNC 872-885/Cit 876-877), GQYEL[R/Cit]VDL[R/Cit]DHGE (SEQ ID NO: 3) [Peptide 56] (TNC 2068-2081/Cit 2073, 2077), and YEL[R/Cit]VDL[R/Cit]D (SEQ ID NO: 4) [Peptide 56B] (TNC 2070-2078/Cit 2073, 2077). In certain embodiments the antigen-specific T cell response is MHC class II molecule-restricted and the MHC class II molecule is HLA-DRB1*04:01.

In certain embodiments there is provided an antigen-pulsed antigen-presenting cell comprising an antigen-presenting cell having an antigen-presenting cell surface on which is presented the above described citrullinated oligopeptide polypeptide capable of being specifically recognized by tenascin C-specific T cells.

In certain embodiments there is provided a method of generating tenascin C-specific T cells, the method comprising contacting in vitro the above described antigen-pulsed antigen-presenting cell with one or a plurality of immunocompatible T-cells, under conditions and for a time sufficient to generate tenascin C-specific T-cells. In certain embodiments the tenascin C-specific T cells are CD4+ T cells. In certain embodiments the CD4+ T cells are regulatory T (Treg) cells. In certain embodiments the method further comprises expanding in vitro the tenascin C-specific CD4+ Treg-cells to obtain one or more clones of said Treg cells in amounts sufficient for T-cell receptor structural characterization; and determining a T-cell receptor (TCR) polypeptide-encoding nucleic acid sequence for one or more of said one or more clones. In certain embodiments the method further comprises transfecting or transducing a T-cell population in vitro with a polynucleotide having said T-cell receptor polypeptide-encoding nucleic acid sequence so-determined, thereby obtaining a population of engineered tenascin C-specific T-cells in an amount effective to adoptively transfer an antigen-specific T-cell response to human tenascin C when administered to the subject. In certain embodiments the T-cell population that is transfected or transduced comprises Treg cells and the antigen-specific T-cell response comprises antigen-specific immunosuppression. In certain embodiments the Treg cells comprise T cells that have been artificially induced to express a Treg phenotype. In certain embodiments the T cells that have been artificially induced to express a Treg phenotype have been engineered to express FOXP3.

In certain other embodiments there is provided a method for treating a condition characterized by a tenascin C-specific autoimmune response, comprising administering to a subject in need thereof a therapeutically effective amount of the engineered tenascin C-specific Treg cells generated according to the above described method. In certain embodiments the Treg cells comprise T cells that have been artificially induced to express a Treg phenotype. In certain embodiments the T cells that have been artificially induced to express a Treg phenotype have been engineered to express FOXP3. In certain embodiments the condition characterized by a tenascin C-specific autoimmune response is rheumatoid arthritis.

In certain embodiments there is provided an engineered tenascin C-specific Treg cell comprising at least one recombinant expression vector encoding a T-cell receptor polypeptide that specifically binds in a human class II HLA-restricted manner to a citrullinated oligopeptide of no more than 18, 17, 16, 15, 14, 13, 12, 11, 10, or 9 amino acids which comprises at least one arginine residue (R) that is citrullinated ([Cit]), said citrullinated oligopeptide comprising an amino acid sequence that is selected from: IS[R/Cit][R/Cit]GDMSS (SEQ ID NO: 1) [Peptide 17A] (TNC 874-882/Cit 876-877), SLIS[R/Cit][R/Cit]GDMSSNPA (SEQ ID NO: 2) [Peptide 17] (TNC 872-885/Cit 876-877), GQYEL[R/Cit]VDL[R/Cit]DHGE (SEQ ID NO: 3) [Peptide 56]

(TNC 2068-2081/Cit 2073, 2077), and YEL[R/Cit]VDL[R/Cit]D (SEQ ID NO: 4) [Peptide 56B] (TNC 2070-2078/Cit 2073, 2077). In certain embodiments there is provided a method for treating a condition characterized by a tenascin C-specific autoimmune response in a subject, comprising adoptively transferring to the subject an effective amount of the above described engineered tenascin C-specific Treg cell.

In certain embodiments there is provided a method for detecting human tenascin C-specific T cell-mediated immune response activity in a subject, comprising: (a) incubating in vitro (i) a biological sample obtained from the subject, said sample comprising T lymphocytes and antigen-presenting cells, with (ii) an isolated citrullinated oligopeptide of no more than 14, 13, 12, 11, 10, or 9 amino acids, comprising a polypeptide of general formula (I) which comprises at least one arginine residue (R) that is citrullinated ([Cit]): N—X—C(I) wherein [R/Cit] is either arginine or citrullinated arginine, under conditions and for a time sufficient for specific recognition by one or more T cell receptors (TCR) that are present in said T lymphocytes of the citrullinated oligopeptide to stimulate a T cell response activity; and (b) determining a test level of the T cell response activity that is stimulated by specific TCR recognition of the citrullinated oligopeptide and that is greater than a control level of T cell response activity that is stimulated by incubating the biological sample with a non-citrullinated polypeptide comprising the oligopeptide of general formula (I) in which there is no citrullinated arginine residue, and therefrom detecting presence in the biological sample of human tenascin C-specific T cell-mediated immune response activity, wherein in general formula (I): (a) X comprises an amino acid sequence that is selected from: IS[R/Cit][R/Cit]GDMSS (SEQ ID NO: 1) [Peptide 17A] (TNC 874-882/Cit 876-877), SLIS[R/Cit][R/Cit]GDMSSNPA (SEQ ID NO: 2) [Peptide 17] (TNC 872-885/Cit 876-877), GQYEL[R/Cit]VDL[R/Cit]DHGE (SEQ ID NO: 3) [Peptide 56] (TNC 2068-2081/Cit 2073, 2077), and YEL[R/Cit]VDL[R/Cit]D (SEQ ID NO: 4) [Peptide 56B] (TNC 2070-2078/Cit 2073, 2077); (b) N is an amino terminus of the oligopeptide and consists of 0, 1, 2, 3, 4, or 5 amino acids that are independently selected from natural amino acids and non-natural amino acids; and (c) C is a carboxy terminus of the oligopeptide and consists of 0, 1, 2, 3, 4, or 5 amino acids that are independently selected from natural amino acids and non-natural amino acids. In certain embodiments, in the citrullinated oligopeptide the amino acid sequence of X in general formula (I) comprises two arginine residues that are citrullinated. In certain embodiments the subject has or is suspected of having rheumatoid arthritis. In certain embodiments the biological sample comprises blood, plasma, serum, lymph, synovial fluid, saliva, sputum, stool, or bronchoalveolar lavage (BAL) fluid. In certain embodiments the T cell response activity is a T regulatory (Treg) cell activity. In certain embodiments the Treg cell activity is selected from Treg cell proliferation, Treg suppression of antigen-stimulated T cell proliferation, Treg suppression of antigen-independent T cell proliferation, release of at least one Treg cytokine, and CD25 (IL2R) upregulation. In certain embodiments the Treg cytokine is selected from IFNγ, IL-10, IL-17, and TGFβ.

In certain embodiments there is provided an isolated citrullinated oligopeptide of no more than 18, 17, 16, 15, 14, 13, 12, 11, 10, or 9 amino acids, comprising a polypeptide of general formula (I) which comprises at least one arginine residue (R) that is citrullinated ([Cit]): N—X—C(I) said citrullinated oligopeptide being capable of eliciting an antigen-specific T cell response to human tenascin-C, wherein [R/Cit] is either arginine or citrullinated arginine and wherein: (a) X comprises an amino acid sequence that is selected from: IS[R/Cit][R/Cit]GDMSS (SEQ ID NO: 1) [Peptide 17A] (TNC 874-882/Cit 876-877), SLIS[R/Cit][R/Cit]GDMSSNPA (SEQ ID NO: 2) [Peptide 17] (TNC 872-885/Cit 876-877), GQYEL[R/Cit]VDL[R/Cit]DHGE (SEQ ID NO: 3) [Peptide 56] (TNC 2068-2081/Cit 2073, 2077), and YEL[R/Cit]VDL[R/Cit]D (SEQ ID NO: 4) [Peptide 56B] (TNC 2070-2078/Cit 2073, 2077); (b) N is an amino terminus of the oligopeptide and consists of 0, 1, 2, 3, 4, or 5 amino acids that are independently selected from natural amino acids and non-natural amino acids; and (c) C is a carboxy terminus of the oligopeptide and consists of 0, 1, 2, 3, 4, or 5 amino acids that are independently selected from natural amino acids and non-natural amino acids.

In certain embodiments there is provided an isolated polynucleotide comprising a nucleic acid sequence that encodes the above described polypeptide of no more than 14, 13, 12, 11, 10, or 9 amino acids of general formula (I) wherein [R/Cit] is arginine, or an isolated polynucleotide comprising a nucleic acid sequence that encodes the above described polypeptide of no more than 18, 17, 16, 15, 14, 13, 12, 11, 10, or 9 amino acids of general formula (I) wherein [R/Cit] is arginine. In another embodiment there is provided an expression vector comprising any of the above described polynucleotides. In certain further embodiments there is provided a host cell transformed or transfected with the expression vector. In certain embodiments there is provided a method of producing either (i) the above described polypeptide of no more than 14, 13, 12, 11, 10, or 9 amino acids of wherein [R/Cit] is arginine or (ii) the above described polypeptide of no more than 18, 17, 16, 15, 14, 13, 12, 11, 10, or 9 amino acids of wherein [R/Cit] is arginine, said method comprising the steps of: a) culturing the above described host cell under conditions and for a time sufficient to permit expression of the polypeptide; and b) isolating the polypeptide from the cultured host cell. In certain embodiments there is provided an isolated polynucleotide that is selected from: (a) an isolated antisense polynucleotide comprising a nucleic acid sequence that is complementary to the above described polynucleotide, (b) an isolated small interfering RNA (siRNA) polynucleotide that is capable of substantially silencing, and is complementary to a region of at least 18 and no more than 42 contiguous nucleotides in, a nucleic acid which encodes any of the above described polypeptides of general formula (I), and (c) an isolated ribozyme that specifically binds to the above described polynucleotide.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE SEVERAL VIEWS OF THE DRAWINGS

FIG. 1 provides a schematic for a systematic discovery approach to identify novel citrullinated tenascin C epitopes that elicit T and B cell responses in patients with RA.

FIG. 2 shows amino acid sequences of cit-TNC peptides (SEQ ID NOS: 3, 17-24) tested for their capacity to bind HLA-DRB1*04:01 in a peptide competition assay.

FIG. 3A shows populations of tetramer+ T cells following expansion in response to eight cit-TNC peptides or the positive control influenza peptide MP 97-116 in a single RA subject. In vitro cultures with greater than 0.1% tetramer positive CD4+ T cells were considered positive. FIG. 3B shows representative positive staining from five different RA subjects for each of the five cit-TNC peptides that were found to be immunogenic.

FIG. 4A shows a peptide competition assay between cit-TNC and arg-TNC peptides to evaluate the role of citrulline residues in immunogenicity and HLA-DRB1*04:01 binding. The EC50 cutoff for measurable binding was 50 μM. X stands for one citrullinated amino acid in cit-TNC, R stands for one arginine amino acid in arg-TNC. FIG. 4B shows a Chi-square test and representative plots showing a representative CD4 T-cell response to cit-TNC or arg-TNC peptides in RA patients or healthy controls (HC) subjects.

FIG. 5 shows the effect of citrullination on the ability of TNC peptides (SEQ ID NOS: 25-40) to bind HLA-DRB1*04:01 and to elicit an immune response in vitro.

FIG. 6 shows clinical characteristics of cohorts referenced in the experimental data, including the T cell cohort, Autoantibody Cohort 1, Autoantibody cohort 2, and Synovial fluid cohort.

FIG. 7 shows frequency and phenotype of tetramer+ cit-TNC-specific T cells as determined ex vivo using a multiplex HLA class II tetramer approach including cell surface marker antibodies to define the phenotype. Frequencies are expressed as the number of antigen-specific cells per million memory CD4+ T cells. Each symbol represents an individual subject (n=7 for HC, n=9 for RA), and the horizontal bar shows the median. FIG. 7A shows the frequency of combined cit-TNC-specific CD4+ memory T cells in patients with RA compared to HC subjects and as positive control, influenza-specific memory CD4+ T cells. FIG. 7B shows the frequency of cit-TNC-specific CD4 memory T cells for each of five cit-TNC peptides. FIG. 7C shows the cit-TNC T cell frequency for each epitope in each subject. Each row stands for a single epitope and each column stands for one RA subject; intensity is based on the number of cit-TNC T cells per million memory CD4 T cells. FIG. 7D shows the frequency of CCR4+, CCR6+ and CD38+ cit-TNC-specific CD4 memory T cells in patients with RA compared to HC subjects. FIG. 7E shows the percent of CD38+ cells among total cit-TNC-specific CD4 memory T in patients with RA compared to HC subjects. FIG. 7F shows the lineage of cit-TNC-specific memory CD4+ T cells in RA subjects, cell populations are defined as: Th2 (CCR4+CCR6-CXCR3−), Th17 (CCR4+CCR6+CXCR3−), and Th1 (CXCR3+CCR4-CCR6−). P-values were calculated using an unpaired non-parametric Mann-Whitney test.

FIG. 8 shows the frequency of cTNC-specific CD4+ memory T-cells as measured directly ex vivo from the peripheral blood of patients with RA and healthy subjects.

FIG. 9 shows sequences of citrullinated peptides (SEQ ID NOS: 41-50) previously reported as RA autoantigens. Peptides derived from α-enolase, fibrinogen, vimentin and CILP that were included as reference epitopes in the FluoroSpot assay are shown.

FIG. 10 shows IFN-γ production from RA patient-derived SFMCs following stimulation with cit-TNC peptides for 48 hours. Synovial fluid mononuclear cells (SFMC) from patients with RA (n=7) were stimulated with cit-TNC peptides or their arginine (arg) counterparts, or other known cit-epitopes: cit-enolase (eno), cit-fibrinogen (Fib), cit-vimentin (vim), cit-cartilage intermediate layer protein (CILP) for 48 hours. Influenza peptide (MP97-116) and anti-CD3 were used as positive controls. IFN-γ was measured by a three-color fluorospot. Number of spots were normalized to spots per million cells and spots seen in the unstimulated wells were subtracted from count in the stimulated wells prior to further analyses. Each symbol represents an individual subject, and the horizontal line shows the median. FIG. 10A shows IFN-γ produced in response to cit-TNC17, cit-TNC22, cit-TNC45 and cit-TNC56 peptides in SFMC isolated from patients with RA. FIG. 10B shows levels of IFN-γ produced by SFMC isolated from patients with RA in response to cit-eno, cit-vim and cit-CILP. FIG. 10C shows the effect of HLA-DR blocking antibodies (DR), and HLA-DQ blocking antibodies (DQ) on cit-TNC-stimulated induction of IFN-γ secretion by SFMC from RA patients (n=3). P-values were calculated using a Wilcoxon matched pairs signed ranked test.

FIG. 11 shows IL-10 and IL-17 production from RA patient-derived SFMCs following stimulation with cit-TNC peptides for 48 hours. Synovial fluid mononuclear cells (SFMC) from patients with RA (n=7) were stimulated with cit-TNC peptides or their arginine counterparts for 48 hours. Influenza peptide (MP54) and CD3 (not shown) were used as positive controls. IL-10 (FIG. 11A) and IL-17 (FIG. 11B) production was measured by a three-color fluorospot. Number of spots were normalized to spots per million cells and spots seen in the unstimulated wells were subtracted from count in the stimulated wells prior to further analyses. Each symbol represents an individual subject, and the horizontal line shows the median. FIG. 11C shows a predominant IL-17 response in a RA subject in which four of five cit-TNC peptides induced SFMC to secrete high levels of IL-17, but all peptides failed to induce IFNγ or IL-10 secretion.

FIG. 12 shows amino acid sequences of peptides (SEQ ID NOS: 51-62) used for detection of anti-citrulline peptide antibodies in ELISA.

FIG. 13 shows antibody responses to cit-TNC peptides or their arginine counterparts in serum from patients with RA and HC subjects as measured by ELISA. Numbers below x-axis indicate percentage of each cohort that were seropositive. Each symbol represents an individual subject, and the horizontal line shows the median. FIG. 13A shows detection of antibodies to cit-TNC17, cit-TNC56 and cit-TNC5 in cyclic citrullinated peptide (CCP) positive RA (n=17) and HC subjects (n=24). Each peptide was run on one plate and each peptide pair (arg/cit) was run at the same day. P-values were calculated using a Mann-Whitney test. (**p<0.01, ***p<0.001 and ****p<0.001). FIG. 13B shows correlation of CCP2 antibody levels with cit-TNC17, cit-TNC5, and cit-TNC56 antibody levels (Spearman r test *p<0.05). FIG. 13C shows antibody reactivity to cit-TNC peptides in polyclonal CCP− pools from synovial fluid than plasma. Cutoff for positivity is 1,000, and P-values were calculated using a paired two tailed Student t test. FIG. 13D shows cit-TNC antibody reactivity over time in paired serum and synovial fluid samples from a single RA subject ten years apart. T=time point. The different colors (red, green, blue) in FIG. 13C and FIG. 13D indicate the specific peptides tested in the microarray and correlate with FIG. 14.

FIG. 14 shows sequences of peptides (SEQ ID NOS: 63-71) tested in microarray used in FIG. 13C and FIG. 13D.

FIG. 15 shows cross-reactivity of anti-cit-TNC antibodies. Sera from subjects that were double-reactive for cit-TNC17 and cit-TNC56 antibodies were pre-incubated with increasing concentrations of the indicated peptides, and the IgG response to cit-TNC17, cit-TNC56 and cit-TNC5 were measured. FIG. 15A shows cross-reactivity inhibition data between anti-cit-TNC17 and both anti-cit-TNC56 and anti-cit-TNC5; FIG. 15B shows cross-reactivity inhibition data between anti-cit-TNC56 and both anti-cit-TNC17 and anti-cit-TNC5.

FIG. 16 shows antibody responses to cit-TNC peptides or their arginine counterparts in serum from patients with RA and healthy control (HC) subjects as measured by ELISA. FIG. 16A shows cit-TNC17 and cit-TNC56 seropositivity in CCP+RA (n=55) and CCP− RA subjects (n=43). Each symbol represents an individual subject, and the horizontal line shows the median. Dotted lines indicate cut-off for positivity. Numbers below x-axis indicate percentage of each cohort that were seropositive. Left panel, cit-TNC17, cut off AU=29.51; Right panel, cit-TNC56, cut off AU=91.98. P-values were calculated using a Kruskal Wallis test with Dunn's multiple comparison test; (*p<0.05 and ****p<0.001). Heatmaps in FIG. 16B show cit-TNC antibody seropositivity and its association with the HLA shared epitope (SE) in CCP+RA subjects (n=72; Autoantibody Cohorts 1 and 2 combined) and CCP− RA patients (n=43).

FIG. 17 shows the association of cit-TNC antibodies with clinical measures and smoking in seropositive rheumatoid arthritis patients (FIG. 17A), and in only those positive for the shared epitope (FIG. 17B).

FIG. 18 shows the full-length human tenascin-C (TNC) amino acid sequence (www-ncbi-nlm-nih-gov/protein/CAA55309.1) (SEQ ID NO: 72) and (SEQ ID NOS: 73-78).

### DETAILED DESCRIPTION

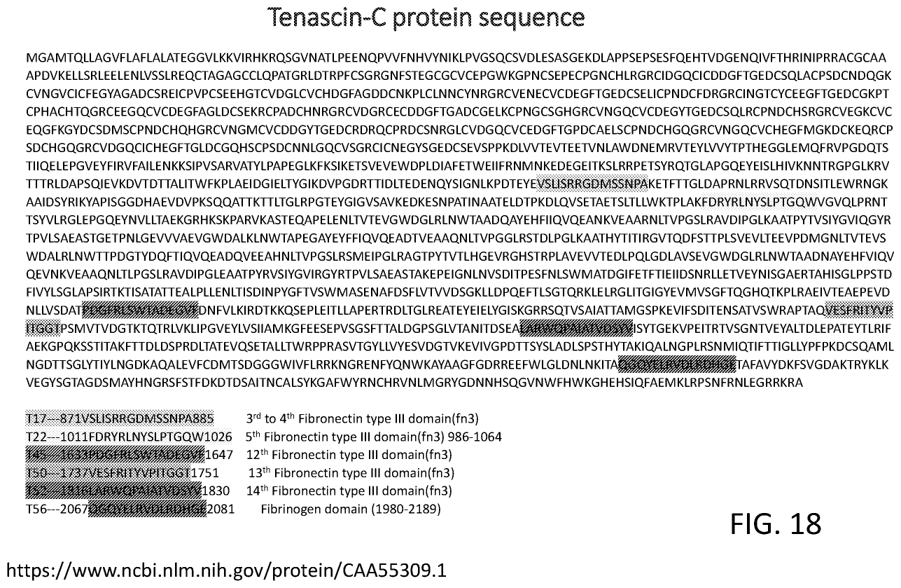

Embodiments of the present invention, as disclosed herein, relate to oligopeptides from the human extracellular matrix protein tenascin-C (TNC, FIG. 18; GenBank: CAA55309.1; EMBL Acc. X78565.1; Gherzi et al., 1995 J. Biol. Chem. 270:3429; cf. NP_002151.2) that specifically bind to MHC class II molecules, and in particular, to citrullinated forms of such TNC-derived oligopeptides that are capable of eliciting autoimmune responses in subjects with rheumatoid arthritis (RA). A subset of the disclosed oligopeptides display increased immunogenicity when citrullinated and surprisingly, are recognized by both CD4+ T-cells and circulating antibodies derived from subjects with RA. Affinity maturation and somatic maturation are known to occur in RA autoantibodies (e.g., antibodies that react with self-antigens), and moreover, infiltrating T-cells are commonly found in joints of RA patients. Together, these findings suggest collaboration between T-cells and B-cells to amplify autoimmunity in RA pathogenesis. The oligopeptides described herein are thus remarkable for establishing a link between the failure of T-cell and B-cell tolerance in RA.

Tenascin-C (TNC) has previously been shown to be associated with disease pathogenesis in RA patients, however, prior to the present disclosure the key immunodominant epitopes targeted by autoantibodies and autoreactive T-cells in RA had not been determined. As described in greater detail below, a previously described algorithm (James et al., 2014 Arthritis Rheumatol. 66:1712; James et al., 2010 Arthritis & Rheumatism. 62:2909) was used to scan the entirety of the full-length human tenascin-C protein sequence in silico. Sixty-four candidate oligopeptides were identified, each with a length of 15 amino acids and predicted to bind HLA-DRB1*04:01, a MHC allele associated with RA risk (Stastny et al., 1978 N Engl J Med. 298:869; Eyre et al., 2012 Nat Genet. 44:1336). Candidate oligopeptides were next evaluated for their capacity to bind HLA-DRB1*04:01 in both non-citrullinated native form and citrullinated form. Eight of sixty-four citrullinated TNC (cit-TNC) peptides bound HLA-DRB1*04:01 with a moderate to high affinity (less than 50 M as measured by a peptide competition binding assay) and were evaluated in vitro for their capacity to elicit antigen-specific immune responses from T-cells. Five oligopeptides were determined to be immunogenic, and of note, two of these oligopeptides contained two arginine sites that were available for potential citrullination, while the remaining three oligopeptides each contained one arginine site. Interestingly, when two arginine sites were available, citrullination of both sites was required in order to render the oligopeptides immunogenic. In subjects with RA, the frequency of CD4+ T-cells specifically reactive with the herein identified immunogenic cit-TNC oligopeptides was increased in peripheral blood as compared to the frequency of such cells in healthy control individuals. More remarkably, the frequency in RA subjects of CD4+ T-cells specific for cit-TNC peptides was higher than the previously reported frequencies of CD4+ T-cells specifically reactive with other citrullinated antigens (e.g., Rims et al., 2019 Arthritis Rheum. 71:518). A significant proportion of cit-TNC-reactive T-cells from RA patients expressed CD38 (a marker of CD4+ T-cell activation), indicating that in RA, cit-TNC-specific CD4+ T-cells are activated and proliferating in vivo. In addition, the cytokine response to cit-TNC oligopeptides in immune cells derived from RA patient synovial fluid was an order of magnitude greater than that which was observed for other citrullinated autoantigens (e.g., self-derived antigens that activate immune effector pathways) that were previously implicated in RA pathogenesis (e.g., Law et al., 2012 Arthritis Res. Ther. 14:R118). Collectively, the present disclosure demonstrates the importance of citrullinated tenascin-C as an autoantigen in RA and establishes several cit-TNC oligopeptides as a link between epitopes driving T and B cell responses in the context of the HLA risk allele DRB1*04:01.

The compositions and methods described herein will, in certain embodiments, have therapeutic utility for the detection, characterization, and treatment of diseases and conditions associated with the expression of cit-TNC autoantigenic peptides (e.g., detectable cit-TNC autoantigenic peptides and/or detectable cit-TNC-specific autoantibodies or cit-TNC-specific autoreactive T cells, detected at a level that is greater in magnitude, in a statistically significant manner, than the level, respectively, of cit-TNC auto-antigenic peptides, cit-TNC-specific autoantibodies and/or cit-TNC-specific autoreactive T cells that is detectable in a normal or disease-free subject). Such diseases include rheumatoid arthritis (RA), ulcerative colitis, Crohn's disease, systemic lupus erythematosus, ankylosing spondylitis, and other autoimmune diseases and include without limitation, diseases that arise from the presence of citrullinated tenascin-C autoantigenic peptides. Non-limiting examples of these and related uses are described herein.

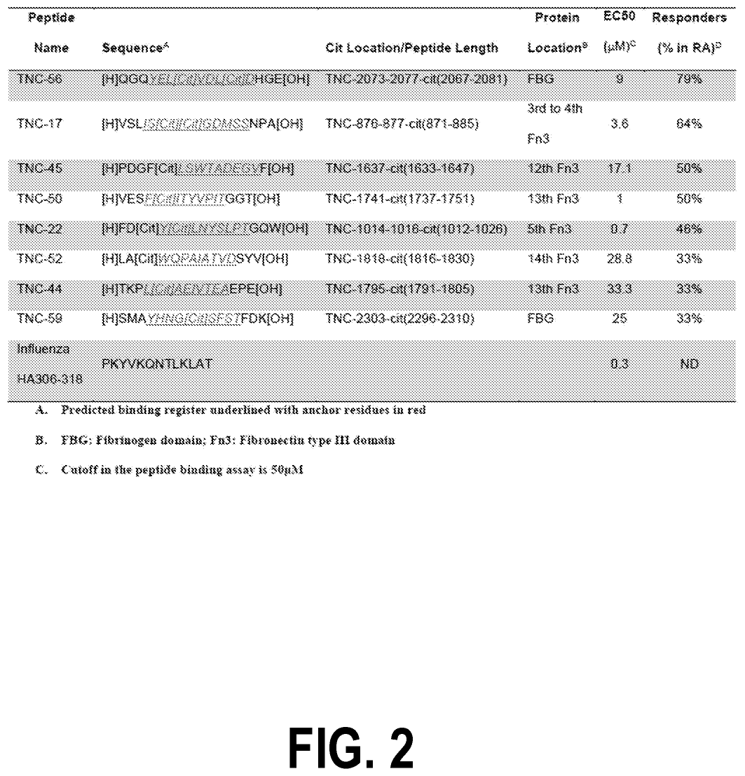

According to certain embodiments described herein, the presently disclosed compositions and methods may be used for in vitro and in vivo stimulation of cit-TNC antigen-specific T-cell responses. Certain embodiments thus contemplate, for example, the use of the herein disclosed immunogenic cit-TNC peptides in peptide-based vaccines, in particle-based peptide vaccines, and in antigen-pulsed antigen-presenting cells (APCs) for APC-based vaccines, such as tolerogenic vaccines. Certain other embodiments, for example, contemplate the use of the herein disclosed immunogenic cit-TNC peptides or larger cit-TNC fragments (e.g., FIGS. 2, 5, 9, 12, 14) or of the whole cit-TNC protein (e.g., FIG. 18) to induce T-cell responses in vitro or in vivo. Antigen-specific T cells, for example, immunomodulatory antigen-specific regulatory (Treg) T-cells induced by in vitro stimulation with cit-TNC oligopeptides, may also according to certain embodiments be directly administered as an immunotherapeutic agent by adoptive transfer. Also contemplated are embodiments in which functional (e.g., productively rearranged) cit-TNC-recognizing T-cell receptor (TCR)-encoding genes may be identified and cloned for use in transfecting/transducing an immunotherapeutic, cit-TNC-specific T-cell population (e.g., Treg cells) for adoptive transfer into subjects having RA or other conditions characterized by an inappropriate or deleterious tenascin C-specific autoimmune response. The TNC peptides of the current disclosure, including in certain preferred embodiments citrullinated TSC peptides, also contain epitopes that drive B cell responses and thus may be utilized for the detection and quantification of antibodies that specifically bind tenascin-C peptides (including in certain preferred embodiments antibodies specific for citrullinated TSC peptides) in biological samples from subjects having, suspected of having, or being at risk for having an autoimmune disease (e.g., RA) or other condition characterized by a TNC-specific autoimmune response.

Self-peptide vaccination to suppress autoimmunity is described, for example, in Ruiz et al., 1999 J. Immunol. 162: 3336-41; O'Herrin et al., 2001 J.

Immunol. 167: 2555-60; Gabrysova et al., 2010 Eur. J. Imunol. 40: 1386-95; Wegmann et al., 2008 J. Immunol. 181: 3301-9; Muller et al., 1998 J. Allergy Clin. Immunol. 101(6 Pt 1): 747-54. Accordingly and as disclosed herein, certain contemplated embodiments relate to uses of the presently described compositions and methods to induce tolerance in an antigen-specific manner, for example, by antigen-specifically suppressing T cell and/or antibody responses to cit-TNC or to any of the herein described TNC oligopeptides including citrullinated TNC oligopeptides.

In certain embodiments, presently disclosed immunogens for use in inducing, eliciting or detecting cit-TNC-specific immune responses (including immunomodulatory responses), or in inducing, eliciting or detecting cit-TNC-specific binding of an antibody (including an autoantibody) directed against the herein described cit-TNC polypeptides, may include a citrullinated oligopeptide of no more than 14, 13, 12, 11, 10, or 9 amino acids wherein the polypeptide comprises a sequence of at least 9, 10, 11, 12, 13, or 14 continuous amino acids from the TNC sequence set forth in SEQ ID NO: 72 (FIG. 18). According to certain presently disclosed embodiments, an isolated citrullinated oligopeptide of no more than 14, 13, 12, 11, 10, or 9 amino acids, comprises a polypeptide of general formula (I) which comprises at least one arginine residue (R) that is citrullinated ([Cit]):

N—X—C (I)

said citrullinated oligopeptide being capable of eliciting an antigen-specific T-cell response to human tenascin-C specific or specifically binding an antibody, wherein [R/Cit] is either arginine or citrullinated arginine and wherein,
(a) X comprises an amino acid sequence that is selected from:

| | [Peptide 17A] |
|---|---|
| (SEQ ID NO: 1) | |
| | IS[R/Cit][R/Cit]GDMSS |
| | (TNC 874-882/Cit 876-877), |
| | [Peptide 17] |
| (SEQ ID NO: 2) | |
| | SLIS[R/Cit][R/Cit]GDMSSNPA |
| | (TNC 872-885/Cit 876-877), |
| | [Peptide 56] |
| (SEQ ID NO: 3) | |
| | GQYEL[R/Cit]VDL[R/Cit]DHGE |
| | (TNC 2068-2081/Cit 2073, 2077), |
| | and |
| | [Peptide 56B] |
| (SEQ ID NO: 4) | |
| | YEL[R/Cit]VDL[R/Cit]D |
| | (TNC 2070-2078/Cit 2073, 2077); |

(b) N is an amino terminus of the oligopeptide and consists of 0, 1, 2, 3, 4, or 5 amino acids that are independently selected from natural amino acids and non-natural amino acids; and
(c) C is a carboxy terminus of the oligopeptide and consists of 0, 1, 2, 3, 4, or 5 amino acids that are independently selected from natural amino acids and non-natural amino acids.

Table 1 discloses exemplary cit-TNC oligopeptides having the general formula (I) N—X—C and a total length of 14 amino acids. Representative species of isolated citrullinated oligopeptides of no more than 14, 13, 12, 11, 10, or 9 amino acids comprising a polypeptide of general formula (I) N—X—C are contained within the sequences disclosed in Table 1.

TABLE 1
Exemplary Cit-TNC oligopeptides having
a general formula N-X-C and a length of
14 amino acids. Component X is denoted
by underlined, bolded text.

| | oligo- | | | |
|---|---|---|---|---|
| SEQ | peptide | | | Citrul- |
| ID | size | N-X-C | Location | lination |
| 3 | 14mer | GQYEL[R/Cit]VDL | 2068-2081 | 2073, 2077 |
| | | [R/Cit]DHGE | | |
| 5 | 14mer | [R/Cit]VDL[R/Cit] | 2073-2086 | 2073, 2077 |
| | | DHGETAFAV | | |
| 6 | 14mer | ITAQGQYEL[R/Cit] | 2064-2077 | 2073, 2077 |
| | | VDL[R/Cit] | | |
| 7 | 14mer | QYEL[R/Cit]VDL | 2069-2082 | 2073, 2077 |
| | | [R/Cit9 DHGET | | |
| 8 | 14mer | VSLIS[R/Cit][R/Cit] | 871-884 | 876, 877 |
| | | GDMSSNP | | |
| 9 | 14mer | [R/Cit][R/Cit] | 876-889 | 876, 877 |
| | | GDMSSNPAKETF | | |
| 10 | 14mer | DTEYEVSLIS[R/Cit] | 866-879 | 876, 877 |
| | | [R/Cit]GD | | |
| 11 | 14mer | VSLIS[R/Cit][R/Cit] | 871-884 | 876, 877 |
| | | GDMSSNP | | |

In certain other embodiments, presently disclosed immunogens for use in inducing, eliciting or detecting cit-TNC-specific immune responses (including immunomodulatory responses), or in inducing, eliciting or detecting cit-TNC-specific binding of an antibody (including an autoantibody) directed against the herein described cit-TNC polypeptides, may include an oligopeptide of no more than 18, 17, 16, 15, 14, 13, 12, 11, 10, or 9 amino acids wherein the polypeptide comprises a sequence of at least 9, 10, 11, 12, 13, 14, 15, 16, 17 or 18 continuous amino acids from the TNC sequence set forth in SEQ ID NO: 72 (FIG. 18) [CAA55309.1]. According to certain presently disclosed embodiments an isolated citrullinated oligopeptide of no more than 18, 17, 16, 15, 14, 13, 12, 11, 10, or 9 amino acids, comprises a polypeptide of general formula (I) which comprises at least one arginine residue (R) that is citrullinated ([Cit]):

N—X—C (I)

said citrullinated oligopeptide being capable of eliciting an antigen-specific T-cell response to human tenascin-C specific or specifically binding an antibody, wherein [R/Cit] is either arginine or citrullinated arginine and wherein,
(a) X comprises an amino acid sequence that is selected from:

| | [Peptide 17A] |
|---|---|
| (SEQ ID NO: 1) | |
| | IS[R/Cit][R/Cit]GDMSS |
| | (TNC 874-882/Cit 876-877), |
| | [Peptide 17] |
| (SEQ ID NO: 2) | |
| | SLIS[R/Cit][R/Cit]GDMSSNPA |
| | (TNC 872-885/Cit 876-877), |
| | [Peptide 56] |
| (SEQ ID NO: 3) | |
| | GQYEL]R/Cit]VDL[R/Cit]DHGE |
| | (TNC 2068-2081/Cit 2073, 2077), |
| | and |
| | [Peptide 56B] |
| (SEQ ID NO: 4) | |
| | YEL[R/Cit]VDL[R/Cit]D |
| | (TNC 2070-2078/Cit 2073, 2077); |

(b) N is an amino terminus of the oligopeptide and consists of 0, 1, 2, or 3 amino acids that are independently selected from natural amino acids and non-natural amino acids; and
(c) C is a carboxy terminus of the oligopeptide and consists of 0, 1, 2, or 3, amino acids that are independently selected from natural amino acids and non-natural amino acids.

Table 2 discloses exemplary cit-TNC oligopeptides having a general formula N—X—C and a total length 18 amino acids. Representative species of isolated citrullinated oligopeptides of no more than 18, 17, 16, 15, 14, 13, 12, 11, 10, or 9 amino acids comprising a polypeptide of general formula (I) N—X—C are contained within the sequences disclosed in Table 2.

TABLE 2
Exemplary Cit-TNC oligopeptides having
a general formula N-X-C and a length of
18 amino acids. Component X is denoted
by underlined, bolded text.

| | oligo- | | | |
|---|---|---|---|---|
| SEQ | peptide | | | Citrul- |
| ID | size | N-X-C | Location | lination |
| 12 | 18mer | EYEVSLIS[Cit] | 868-885 | 876, 877 |
| | | [Cit]GDMSSNPA | | |
| 13 | 18mer | VSLIS[Cit][Cit] | 871-888 | 876, 877 |
| | | GDMSSNPAKET | | |
| 14 | 18mer | TAQQGQYEL[Cit] | 2064-2081 | 2073, 2077 |
| | | VDL[Cit]DHGE | | |
| 15 | 18mer | QGQYEL[Cit]VDL | 2067-2084 | 2073, 2077 |
| | | [Cit]DHGETAF | | |

In certain related embodiments a citrullinated oligopeptide of no more than 18, 17, 16, 15, 14, 13, 12, 11, 10, or 9 amino acids comprising a polypeptide of general formula (I) N—X—C may comprise any of the amino acid sequences shown in FIGS. 2, 5, 9, 12, and 14.

Accordingly, in these and other embodiments it will be appreciated that the amino terminus of certain TNC-derived peptides disclosed herein as comprising immunogenic epitopes may consist of 0-5 independently selected natural or non-natural amino acids, and/or that in certain embodiments the carboxy terminus of certain such peptides may consist of 0-5 independently selected natural or non-natural amino acids, where such amino and carboxy termini may have any sequence so long as the isolated peptide is of no more than 9-14 amino acids (or in certain other embodiments no more than 9-18 amino acids) and comprises N—X—C as recited herein, and is capable of specifically eliciting an antigen-specific T-cell response to human tenascin-C (preferably to citTNC, and preferably in a cit-dependent manner) or specifically binding to a human tenascin-C-specific antibody (which is preferably specific for citTNC, and preferably in a cit-dependent manner). Citrullination-dependent T-cell or antibody recognition of herein described TNC-derived oligopeptides may be demonstrated by detecting a level of T-cell or antibody recognition of a citrullinated TNC-derived oligopeptide as described herein, which detectable level is significantly increased (e.g., in a statistically significant manner relative to an appropriate control) for the citrullinated TNC-derived oligopeptide relative to the level that is detectable for a corresponding TNC-derived peptide that is hypocitrullinated (i.e., lacks at least one or more citrullinated arginines compared to the citTNC oligopeptide, and which may comprise no citrullinated argnines at all).

### Polypeptides and Oligopeptides

The terms “protein”, “peptide”, and “polypeptide” are used interchangeably and mean a polymer of amino acids not limited to any particular length. The terms “peptide” and “polypeptide” shall include “oligopeptides”. The term “oligopeptide” as used herein refers to a series of amino acid residues, which are usually connected to each other by a peptide bond between an alpha amino acid adjacent to the amino acid and a carbonyl group. Oligopeptides usually have a length of less than about 30 amino acid residues and may comprise, for example, 9, 10, 11, 12, 13, 14, 15, 16, 17, 18, 19, 20, 21, 22, 23, 24, 25, 26, 27, 28, or 29 amino acids. The terms “protein”, “peptide”, “polypeptide”, and “oligopeptide” do not exclude modifications such as myristylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having complete or partial sequences of native proteins, that is, proteins produced naturally, or natural or artificial sequences that are products of genetic engineering, such as may be expressed by recombinant host cells or in cell-free translation systems, or peptides (including oligopeptides) or proteins that are produced by wholly artificial chemical synthesis. A polypeptide or protein may comprise a molecule having the amino acid sequence of a native protein, or having deletions from, additions to, and/or substitutions of one or more amino acids of the native sequence. Thus, a “polypeptide” or a “protein” can comprise one (termed “a monomer”) or a plurality (termed “a multimer”) of amino acid chains, typically in peptide linkage. The terms “protein”, “peptide”, “polypeptide”, and “oligopeptide” specifically encompass the immunomodulatory polypeptides of the present disclosure (e.g., any of the amino acid sequences shown in FIGS. 2, 5, 9, 12, and 14, or any of SEQ ID NOS: 1-4 (Peptides 17, 17A, 56, 56B)).

In some embodiments, the peptides described here are post-translationally modified at arginine residues via a process termed “citrullination” in which a side chain of arginine, peptidyl arginine, is converted to peptidyl citrulline to produce citrulline by calcium-dependent peptidylarginine deiminases (e.g., Bickert et al., 2013 Biopolymers 99:155). As used herein, “citrullinated” and “citrullination” are used interchangeably to refer to the conversion of arginine to citrulline.

In certain preferred embodiments oligopeptides comprising a polypeptide of general formula (I) as disclosed herein in which [R/Cit] is arginine, or any of the amino acid sequences shown in FIGS. 2, 5, 9, 12, and 14 which comprises a citrullinated arginine residue, or any of SEQ ID NOS: 1-4 (Peptides 17, 17A, 56, 56B) that comprises a citrullinated arginine residue, are synthesized either by artificial chemical synthesis or by recombinant expression according to well known methodologies. Artificial citrullination of the polypeptide may then be achieved post-synthetically by reaction under suitable conditions with a protein arginine deiminase (PAD) enzyme as described, for example, by Bickert et al. (2013 Biopolymers 99: 155-163) to obtain a citrullinated oligopeptide in which at least one arginine residue is converted to citrullinated arginine and, in certain preferred embodiments, in which two arginine residues are citrullinated. PAD is readily available from commercial sources (e.g., Sigma-Aldrich, St. Louis, MO) for such purposes.

Alternatively and in certain other contemplated embodiments, isolated fragments of citrullinated tenascin C comprising the herein disclosed citrullinated oligopeptides may be obtained by biochemical isolation from biological samples. As described herein, the presently disclosed citrullinated oligopeptides exhibit markedly different structural and functional properties relative to intact, native tenascin C.

Disclosed herein are a number of representative TNC-derived oligopeptides that comprise N—X—C according to general formula [I] as recited herein, that are capable of specifically eliciting an antigen-specific T-cell response to human TNC and/or binding a TNC-specific antibody. The oligopeptide of general formula [I] N—X—C, may be citrullinated at one or two arginine residues and in a further embodiment, the amino acid sequence of X in general formula [I] may be citrullinated at two arginine residues. According to certain embodiments of the present disclosure, the isolated cit-TNC oligopeptides may have a greater binding affinity for a given MHC class II molecule as compared to the affinity by which its hypocitrullinated counterpart binds to the MHC class II molecule. The term “hypocitrullinated”, as used herein, refers to an oligopeptide wherein at least one or at least two arginine residues that are potentially available for citrullination are not citrullinated. One of skill in the art will recognize, from the present disclosure, that “binding affinity” may refer to the strength of the binding interaction between the TNC-derived oligopeptide and a MHC class II molecule, and may be quantified by conventional techniques, for example, those described by Scatchard et al. (Ann. N. Y. Acad. Sci. USA 51:660 (1949)), or by surface plasmon resonance (SPR; BIACORE™, Biosensor, Piscataway, N.J.), or by any of a number of other established methodologies described in references cited herein for determining the affinity binding constant of interacting, and preferably specifically interacting, components in protein-protein binding events. In certain embodiments, the TSC-derived peptides of the present disclosure, preferably as citrullinated oligopeptides as provided herein, for example, a citrullinated oligopeptide comprising any of the amino acid sequences shown in FIGS. 2, 5, 9, 12, and 14, or in Tables 1 or 2, or any of SEQ ID NOS: 1-4 (Peptides 17, 17A, 56, 56B), may bind specifically to a particular MHC class II allele, which may include HLA-DRB1*04:01. The presently contemplated invention embodiments, however, are not intended to be so limited such that in view of the present disclosure those familiar with the art will be readily able to make and use additional TNC peptides, cit-TNC peptides, and variants thereof that are immunogenic for T-cells, including such cit-TNC oligopeptides that engage in specific binding interactions with other allelic forms of MHC class II molecules.

For example, determination of the three-dimensional structures of representative immunogenic TNC-derived peptides bearing T-cell epitopes as described herein may be made through routine methodologies such that substitution of one or more amino acids with selected natural or non-natural amino acids can be virtually modeled for purposes of determining whether a so derived structural variant retains the space-filling, charge, hydrophilic and/or hydrophobic properties of presently disclosed species, including modeling of potential peptide affinity interactions with MHC peptide-binding grooves (e.g., BIMAS molecular modeling software, described by Parker et al. 1994 J. Immunol. 152:163; Tsites database, Feller et al. 1991 Nature 349:720; Rothbard et al. 1988 EMBO J. 7:93-100; Deavin et al. 1996 Mol. Immunol. 33:145-155; and other HLA peptide binding prediction analyses. See also, for instance, Donate et al., 1994 Prot. Sci. 3:2378; Bradley et al. 2005 Science 309: 1868-1871; Schueler-Furman et al. 2005 Science 310:638; Dietz et al. 2006 Proc. Nat. Acad. Sci. USA 103:1244; Dodson et al., 2007 Nature 450:176; Qian et al., 2007 Nature 450:259; and Raman et al. 2010 Science 327:1014-1018). These and other references describe computer algorithms that may be used for related embodiments, such as for rational design of variants of the TNC-derived peptides bearing T-cell epitopes as provided herein (e.g., SEQ ID NOS:1-4 (Peptides 17, 17A, 56, 56B)), for instance, by allowing for determination of atomic dimensions from space-filling models (van der Waals radii) of energy-minimized conformations.

In view of the present disclosure that the herein described cit-TNC oligopeptides contain immunogenic epitopes, e.g., the molecular structures that are specifically recognized by T-cells via the T-cell receptor (TCR) including via MHC-restricted T-cell recognition, it is thus expressly contemplated that alterations (e.g., increases or decreases that are detectable with statistical significance) in the immunogenicity of any given epitope-bearing cit-TNC peptide may be introduced by structural modification, for example, to obtain immunogenic cit-TNC peptide-derived variants. Means for enhancing the immunogenicity of a peptide-defined epitope are known in the art, and may include the altered peptide ligand (APL) approach by which structural modifications are made to a given peptide. Peptide variants of enhanced immunogenicity have been generated as APLs, as described in other antigen systems, for instance, by Abdul-Alim et al. 2010 J. Immunol. 184:6514; Douat-Casassus et al. 2007 J. Med. Chem. 50:1598; Carrabba et al. 2003 Canc. Res. 63:1560; and Shang et al. 2009 Eur. J. Immunol. 39:2248. Accordingly it will be appreciated from the present disclosure that TNC peptide sequences include a large number of immunogenic epitopes for T-cells, such that TNC fragments (e.g., in certain preferred embodiments, sequences of at least 9, 10, 11, 12, 13, or 14 contiguous amino acids comprising the TNC-derived amino acid sequences set forth in SEQ ID NOS:1-4 (Peptides 17, 17A, 56, 56B) or any of the amino acid sequences shown in FIGS. 2, 5, 9, 12, and 14 and Tables 1 and 2; and in certain other preferred embodiments sequences of at least 9, 10, 11, 12, 13, 14, 15, 16, 17, or 18 contiguous amino acids comprising the TNC-derived amino acid sequences set forth in SEQ ID NOS:1-4 (Peptides 17, 17A, 56, 56B) or any of the amino acid sequences shown in FIGS. 2, 5, 9, 12, and 14 and Tables 1 and 2, and/or variants as provided herein (including APLs) may be encompassed within certain embodiments.

Some additional non-limiting examples of computer algorithms that may be used for these and related embodiments, such as for rational design of variants of the herein described cit-TNC immunogenic peptide epitopes (e.g., SEQ ID NOS:1-4, peptides 17, 17A, 56, 56B), include NAMD, a parallel molecular dynamics code designed for high-performance simulation of large biomolecular systems, and VMD which is a molecular visualization program for displaying, animating, and analyzing large biomolecular systems using 3-D graphics and built-in scripting (see Phillips et al., 2005 Journal of Computational Chemistry 26:1781; Humphrey et al., “VMD—Visual Molecular Dynamics”, J. Molec. Graphics, 1996, vol. 14, pp. 33-38; see also the website for the Theoretical and Computational Biophysics Group, University of Illinois at Urbana-Champagne, at ks.uiuc.edu/Research/vmd/). Many other computer programs are known in the art and available to the skilled person and allow for determining atomic dimensions from space-filling models (van der Waals radii) of energy-minimized conformations; for example, GRID, which seeks to determine regions of high affinity for different chemical groups, thereby enhancing binding; Monte Carlo searches, which calculate mathematical alignment; and CHARMM (Brooks et al., 1983 J. Comput. Chem. 4:187) and AMBER (Weiner et al., 1981 J. Comput. Chem. 106: 765), which assess force field calculations, and analysis (see also, Eisenfield et al., 1991 Am. J. Physiol. 261:C376; Lybrand 1991 J. Pharm. Belg. 46:49; Froimowitz 1990 Biotechniques 8:640; Burbam et al., 1990 Proteins 7:99; Pedersen 1985 Environ. Health Perspect. 61:185; and Kini et al., 1991 J. Biomol. Struct. Dyn. 9:475). A variety of appropriate computational computer programs are also commercially available, such as from Schrödinger (Munich, Germany) or Cyrus Biotechnology (Seattle, WA).

“Natural or non-natural amino acid” includes any of the common naturally occurring amino acids which serve as building blocks for the biosynthesis of peptides, polypeptides and proteins (e.g., alanine, cysteine, aspartic acid, glutamic acid, phenylalanine, glycine, histidine, isoleucine, lysine, leucine, methionine, asparagine, proline, glutamine, arginine, serine, threonine, valine, tryptophan, tyrosine) and also includes modified, derivatized, enantiomeric, rare and/or unusual amino acids, whether naturally occurring or synthetic, for instance, hydroxyproline, hydroxylysine, desmosine, isodesmosine, ε-N-methyllysine, ε-N-trimethyllysine, methylhistidine, dehydrobutyrine, dehydroalanine, α-aminobutyric acid, β-alanine, γ-aminobutyric acid, homocysteine, homoserine, citrulline, ornithine and other amino acids that may be isolated from a natural source and/or that may be chemically synthesized, for instance, as may be found in Proteins, Peptides and Amino Acids Sourcebook (White, J. S. and White, D. C., 2002 Humana Press, Totowa, NJ) or in Amino Acid and Peptide Synthesis (Jones, J., 2002 Oxford Univ. Press USA, New York) or in Unnatural Amino Acids, ChemFiles Vol. 1, No. 5 (2001 Fluka Chemie GmbH; Sigma-Aldrich, St. Louis, MO) or in Unnatural Amino Acids II, ChemFiles Vol. 2, No. 4 (2002 Fluka Chemie GmbH; Sigma-Aldrich, St. Louis, MO). Additional descriptions of natural and/or non-natural amino acids may be found, for example, in Kotha, 2003 Acc. Chem. Res. 36:342; Maruoka et al., 2004 Proc. Nat. Acad. Sci. USA 101:5824; Lundquist et al., 2001 Org. Lett. 3:781; Tang et al., 2002 J. Org. Chem. 67:7819; Rothman et al., 2003 J. Org. Chem. 68:6795; Krebs et al., 2004 Chemistry 10:544; Goodman et al., 2001 Biopolymers 60:229; Sabat et al., 2000 Org. Lett. 2:1089; Fu et al., 2001 J. Org. Chem. 66:7118; and Hruby et al., 1994 Meths. Mol. Biol. 35:249. The standard three-letter abbreviations and 1-letter symbols are used herein to designate natural and non-natural amino acids.

Other non-natural amino acids or amino acid analogues are known in the art and include, but are not limited to, non-natural L or D derivatives (such as D-amino acids present in peptides), fluorescent labeled amino acids, as well as specific examples including O-methyl-L-tyrosine, L-3-(2-naphthyl)alanine, 3-methyl-phenylalanine, 3-idio-tyrosine, O-propargyl-tyrosine, homoglutamine, an O-4-allyl-L-tyrosine, a 4-propyl-L-tyrosine, a 3-nitro-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-acetyl-L-phenylalanine, an m-acetyl-L-phenylalanine, selenomethionine, telluromethionine, selenocysteine, an alkyne phenylalanine, an O-allyl-L-tyrosine, an O-(2-propynyl)-L-tyrosine, a p-ethylthiocarbonyl-L-phenylalanine, a p-(3-oxobutanoyl)-L-phenylalanine, a p-benzoyl-L-phenylalanine, an L-phosphoserine, a phosphonoserine, a phosphonotyrosine, homoproparglyglycine, azidohomoalanine, a p-iodo-phenylalanine, a p-bromo-L-phenylalanine, dihydroxy-phenylalanine, dihydroxyl-L-phenylalanine, a p-nitro-L-phenylalanine, an m-methoxy-L-phenylalanine, a p-iodo-phenylalanine, a p-bromophenylalanine, a p-amino-L-phenylalanine, and an isopropyl-L-phenylalanine, trifluoroleucine, norleucine (“Nie”), D-norleucine (“dNIe” or “D-Nie”), 5-fluoro-tryptophan, para-halo-phenylalanine, homo-phenylalanine (“homo-Phe”), seleno-methionine, ethionine, S-nitroso-homocysteine, thia-proline, 3-thienyl-alanine, homo-allyl-glycine, trifluoroisoleucine, trans and cis-2-amino-4-hexenoic acid, 2-butynyl-glycine, allyl-glycine, para-azido-phenylalanine, para-cyano-phenylalanine, para-ethynyl-phenylalanine, hexafluoroleucine, 1,2,4-triazole-3-alanine, 2-fluoro-histidine, L-methyl histidine, 3-methyl-L-histidine, β-2-thienyl-L-alanine, β-(2-thiazolyl)-DL-alanine, homoproparglyglycine (HPG) and azidohomoalanine (AHA) and the like.

In certain embodiments a natural or non-natural amino acid may be present that comprises an aromatic side chain, as found, for example, in phenylalanine or tryptophan or analogues thereof including in other natural or non-natural amino acids based on the structures of which the skilled person will readily recognize when an aromatic ring system is present, typically in the form of an aromatic monocyclic or multicyclic hydrocarbon ring system consisting only of hydrogen and carbon and containing from 6 to 19 carbon atoms, where the ring system may be partially or fully saturated, and which may be present as a group that includes, but need not be limited to, groups such as fluorenyl, phenyl and naphthyl.

In certain embodiments a natural or non-natural amino acid may be present that comprises a hydrophobic side chain as found, for example, in alanine, valine, isoleucine, leucine, proline, phenylalanine, tryptophan or methionine or analogues thereof including in other natural or non-natural amino acids based on the structures of which the skilled person will readily recognize when a hydrophobic side chain (e.g., typically one that is non-polar when in a physiological milieu) is present. In certain embodiments a natural or non-natural amino acid may be present that comprises a basic side chain as found, for example, in lysine, arginine or histidine or analogues thereof including in other natural or non-natural amino acids based on the structures of which the skilled person will readily recognize when a basic (e.g., typically polar and having a positive charge when in a physiological milieu) is present.

Polypeptides disclosed herein may include L- and/or D-amino acids so long as the biological activity (e.g., tenascin C (TNC)-specific immunogenicity for T-cells, preferably, and in certain embodiments citrullination-dependent citrullinated TNC (Cit-TNC) specific immunogenicity, and TNC-specific binding of antibodies, which in certain preferred embodiments may be Cit-TNC-specific antibody binding, more preferably antibody binding that is dependent on citrullination of the oligopeptide) of the polypeptide is maintained. The isolated TNC-derived polypeptides may comprise in certain embodiments any of a variety of known natural and artificial post-translational or post-synthetic covalent chemical modifications by reactions that may include glycosylation (e.g., N-linked oligosaccharide addition at asparagine residues, O-linked oligosaccharide addition at serine or threonine residued, glycation, or the like), fatty acylation, acetylation, PEGylation, and phosphorylation. Polypeptides herein disclosed may further include analogs, alleles and allelic variants which may contain amino acid deletions, or additions or substitutions of one or more amino acid residues with other naturally occurring amino acid residues or non-natural amino acid residues.

Peptide and non-peptide analogs may be referred to as peptide mimetics or peptidomimetics, and are known in the pharmaceutical industry (Fauchere 1986 J. Adv. Drug Res. 15:29; Evans et al. 1987 J. Med. Chem. 30:1229). These compounds may contain one or more non-natural amino acid residue(s), one or more chemical modification moieties (for example, glycosylation, pegylation, fluorescence, radioactivity, or other moiety), and/or one or more non-natural peptide bond(s) (for example, a reduced peptide bond: —CH₂—NH₂—). Peptidomimetics may be developed by a variety of methods, including by computerized molecular modeling, random or site-directed mutagenesis, PCR-based strategies, chemical mutagenesis, and others.

The term “isolated” means that the material is removed from its original environment (e.g., the natural environment if it is naturally occurring). For example, a naturally occurring nucleic acid or polypeptide present in a living animal is not isolated, but the same polypeptide or nucleic acid, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

Also contemplated is detectable labeling of the herein described peptides and polypeptides, including the presently disclosed citrullinated oligopeptide and the presently described polypeptide of general formula (I), with detectable indicator moieties (sometimes referred to as reporter moieties) such as fluorophores (e.g., FITC, TRITC, Texas Red, etc.). Examples of a broad range of detectable indicators (including colorimetric indicators) that may be selected for specific purposes are described in The Molecular Probes™ Handbook—Eleventh Edition, 2020 ThermoFisher Scientific/Invitrogen, Carlsbad, CA; Haugland, 2002 Handbook of Fluorescent Probes and Research Products—Ninth Ed., Molecular Probes, Eugene, OR; in Mohr, 1999 J. Mater. Chem., 9: 2259-2264; in Suslick et al., 2004 Tetrahedron 60:11133-11138; and in U.S. Pat. No. 6,323,039. (See also, e.g., Fluka Laboratory Products Catalog, 2001 Fluka, Milwaukee, WI; and Sigma Life Sciences Research Catalog, 2000, Sigma, St. Louis, MO.) A detectable indicator may be a fluorescent indicator, a luminescent indicator, a phosphorescent indicator, a radiometric indicator, a detectable particle, a dye, an enzyme, a substrate of an enzyme, an energy transfer molecule, or an affinity label.

Other detectable indicators for use in certain embodiments contemplated herein include affinity reagents such as antibodies, lectins, immunoglobulin Fc receptor proteins (e.g., Staphylococcus aureus protein A, protein G or other Fc receptors), avidin, biotin, other ligands, receptors or counterreceptors or their analogues or mimetics, and the like. For such affinity methodologies, reagents for immunometric measurements, such as suitably labeled antibodies or lectins, may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., 1987 Methods in Enzymology 135:30-65; Harlow and Lane, Antibodies: A Laboratory Manual, 1988 Cold Spring Harbor Laboratory, Cold Spring Harbor, NY; Haugland, Handbook of Fluorescent Probes and Research Products—Ninth Ed., 2002 Molecular Probes, Eugene, OR; The Molecular Probes™ Handbook—Eleventh Edition, 2020 ThermoFisher Scientific/Invitrogen, Carlsbad, CA; Scopes, R. K., Protein Purification: Principles and Practice, 1987, Springer-Verlag, NY; Hermanson, G. T. et al., Immobilized Affinity Ligand Techniques, 1992, Academic Press, Inc., NY; Luo et al., 1998 J. Biotechnol. 65:225 and references cited therein).

### Nucleic Acids and Polynucleotides

Tenascin C (TSC) polypeptides including TSC oligopeptides as provided herein, and encoding nucleic acid molecules and vectors, may be isolated and/or purified, e.g. from their natural environment, in substantially pure or homogeneous form, or, in the case of nucleic acid, free or substantially free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

The present disclosure thus further provides in certain embodiments an isolated nucleic acid encoding tenascin C (TSC) polypeptides including TSC oligopeptides as provided herein (comprising the amino acid sequence set forth in SEQ ID NOS:1-4 (TSC oligopeptides 17, 17A, 56, 56B) or a polypeptide of 9, 10, 11, 12, 13, 14, 15, 16, 17, or 18 amino acids that includes the polypeptide of general formula (I) as described herein which comprises SEQ ID NOS:1-4 (peptides 17, 17A, 56, 56B) in which [R/Cit] is arginine, for example, any of the polypeptides described in the Examples, or a polypeptide comprising any of the amino acid sequences shown in FIG. 2, 5, 9, 12, or 14 or in Tables 1 or 2 in which [R/Cit], Cit, or the amino acid indicated by “X” is arginine that is available for posttranslational citrullination.

Nucleic acids include DNA and RNA. These and related embodiments may include polynucleotides encoding TSC-derived (e.g., comprising a region of 9, 10, 11, 12, 13, or 14 contiguous amino acids from the TSC sequence shown in FIG. 18) oligopeptides and polypeptides as described herein. The term “isolated polynucleotide” as used herein shall mean a polynucleotide of genomic, cDNA, or synthetic origin or some combination thereof, wherein by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence.

Nucleic acid molecules encoding TSC-derived oligopeptides and polypeptides as provided herein, and vectors comprising such nucleic acid molecules, may be artificially generated synthetically from amino acid sequence information disclosed herein in view of the well-known genetic code, or nucleic acid molecules may be isolated and/or purified, e.g. from their natural environment, in substantially pure or homogeneous form, or, in the case of nucleic acid, free or substantially free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Such modifications may include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated, or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

Therefore, according to these and related embodiments, the present disclosure also provides polynucleotides encoding the polypeptides of general formula (I) in which X comprises at least one of SEQ ID NOS:1-4 (TSC peptides 17, 17A, 56, 56B) and in which [R/Cit] is arginine, or the polypeptides comprising any of the amino acid sequences shown in FIG. 2, 5, 9, 12, or 14 or Tables 1 or 2 in which [R/Cit], Cit, or the amino acid indicated by “X” is arginine that is available for posttranslational citrullination, as described herein. In certain embodiments, polynucleotides are provided that comprise some or all of a polynucleotide sequence encoding a peptide as described herein and complements of such polynucleotides.

In other related embodiments, polynucleotide variants may have substantial identity to a polynucleotide sequence encoding a TSC-derived polypeptide described herein. For example, a polynucleotide may be a polynucleotide comprising at least 70% sequence identity, preferably at least 75%, 80%, 85%, 90%, 95%, 96%, 97%, 98%, or 99% or higher, sequence identity compared to a reference polynucleotide sequence such as a sequence encoding a TSC-derived polypeptide described herein (e.g., a polypeptide of general formula (I) in which X is SEQ ID NO: 1-4 (Peptides 17, 17A, 56, 56B) and wherein [R/Cit] is arginine), using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the specific binding affinity of a T cell receptor (TCR) or antibody for a citrullinated form of the oligopeptide encoded by the variant polynucleotide is not substantially diminished relative to the specific binding affinity for the citrullinated form of an oligopeptide encoded by a polynucleotide sequence specifically set forth herein.

In certain other related embodiments, polynucleotide fragments may comprise or consist essentially of various lengths of contiguous stretches of sequence identical to or complementary to a sequence encoding a TSC-derived polypeptide as described herein. For example, polynucleotides are provided that comprise or consist essentially of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 60, 70, 80, 90, 100 or more contiguous nucleotides of a sequences the encodes a herein disclosed TSC-derived polypeptide, or variant thereof, disclosed herein as well as all intermediate lengths there between. It will be readily understood that “intermediate lengths”, in this context, means any length between the quoted values, such as 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of a polynucleotide encoding a TSC-derived polypeptide described herein or at both ends of a polynucleotide encoding such a polypeptide.

In another embodiment, polynucleotides are provided that are capable of hybridizing under moderate to high stringency conditions to a polynucleotide sequence encoding a herein disclosed TSC-derived polypeptide, or variant thereof, provided herein, or a fragment thereof, or a complementary sequence thereof. Hybridization techniques are well known in the art of molecular biology. For purposes of illustration, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60° C.-65° C. or 65° C.-70° C.

As described elsewhere herein, determination of the three-dimensional structures of representative polypeptides (e.g., polypeptides of 9-14 or 9-18 amino acids that comprise one of SEQ ID NOS:1-4 (TSC peptides 17, 17A, 56, or 56B) or any of the amino acid sequences shown in FIG. 2, 5, 9, 12, or 14 or Tables 1 or 2) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. A variety of computer programs are known to the skilled artisan for determining appropriate amino acid substitutions (or appropriate polynucleotides encoding the amino acid sequence) within polypeptide such that, for example, affinity for a cognate TCR or antibody is maintained, or stronger affinity is achieved.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., Unified Approach to Alignment and Phylogenes, pp. 626-645 (1990); Methods in Enzymology vol. 183, Academic Press, Inc., San Diego, CA; Higgins, D. G. and Sharp, P. M., CAB/OS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, CA (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and ×determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that may encode a TSC-derived peptide as described herein, for example, a polypeptide that comprises one of SEQ ID NOS:1-4 (TSC peptides 17, 17A, 56, or 56B), or any of the amino acid sequences shown in FIGS. 2, 5, 9, 12, or 14 or Tables 1 or 2. Some of these polynucleotides bear minimal sequence identity to the nucleotide sequences of the native or original polynucleotide sequences that encode the polypeptides described herein. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment of the invention, a mutagenesis approach, such as site-specific mutagenesis, may be employed for the preparation of variants and/or derivatives of the polypeptides described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the inventors contemplate the mutagenesis of the polynucleotide sequences that encode a TSC-derived polypeptide disclosed herein, or a variant thereof, to alter one or more properties of the encoded polypeptide, such as the binding affinity of the peptide or the variant thereof for a specific TCR or antibody that recognizes the polypeptide, preferably that recognizes such a polypeptide that is a citrullinated oligopepide in a citrullination-dependent manner. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants, recursive sequence recombination, as described in U.S. Pat. No. 5,837,458, may be employed. In this approach, iterative cycles of recombination and screening or selection are performed to “evolve” individual polynucleotide variants encoding polypeptides having, for example, increased binding affinity for specific TCR and/or antibodies, which in preferred embodiments bind to the citrullinated form of the polypeptide in a citrullination-dependent fashion. Certain embodiments also provide constructs in the form of plasmids, vectors, transcription or expression cassettes which comprise at least one polynucleotide as described herein.

According to certain related embodiments there is provided a recombinant host cell which comprises one or more constructs as described herein; a nucleic acid encoding the herein described TSC-derived polypeptide or variant thereof; and a method of producing of the encoded product, which method comprises expression from encoding nucleic acid therefor. Expression may conveniently be achieved by culturing under appropriate conditions recombinant host cells containing the nucleic acid. Following production by expression, an expressed TSC-derived polypeptide may be isolated and/or purified using any suitable technique, and then used as desired.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of peptides in prokaryotic cells such as E. coli is well established in the art. For a review, see for example Pluckthun, A. Bio/Technology 9: 545-551 (1991). Expression in eukaryotic cells in culture is also available to those skilled in the art as an option for production of polypeptides, see reviews, for example Ref, M. E. (1993) Curr. Opinion Biotech. 4: 573-576; Trill J. J. et al. (1995) Curr. Opinion Biotech 6: 553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The term “host cell” is used to refer to a cell into which has been introduced, or which is capable of having introduced into it, a nucleic acid sequence encoding one or more of the herein described TSC-derived polypeptides, and which further expresses or is capable of expressing a selected gene of interest, such as a gene encoding any herein described TSC-derived polypeptide. The term includes the progeny of the parent cell, whether or not the progeny are identical in morphology or in genetic make-up to the original parent, so long as the selected gene is present. Accordingly there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. In one embodiment, the nucleic acid is integrated into the genome (e.g. chromosome) of the host cell. Integration may be promoted by inclusion of sequences which promote recombination with the genome, in accordance—with standard techniques.

The present disclosure also provides, in certain embodiments, a method which comprises using a construct as stated above in an expression system in order to express a particular polypeptide such as a TSC-derived polypeptide (e.g., one comprising one of SEQ ID NOS:1-4, TSC peptides 17, 17A, 56, or 56B) as described herein. The term “transduction” is used to refer to the transfer of genes from one bacterium to another, usually by a phage. “Transduction” also refers to the acquisition and transfer of eukaryotic cellular sequences by retroviruses. The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, MOLECULAR CLONING, A LABORATORY MANUAL, Cold Spring Harbor Laboratories; Davis et al., 1986, BASIC METHODS IN MOLECULAR BIOLOGY, Elsevier; and Chu et al., 1981, Gene 13:197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “naturally occurring” or “native” when used in connection with biological materials such as nucleic acid molecules, polypeptides, host cells, and the like, refers to materials which are found in nature and are not manipulated by a human. Similarly, “non-naturally occurring” or “non-native” as used herein refers to a material that is not found in nature or that has been structurally modified or synthesized by a human.

Certain embodiments contemplated herein include antisense-based nucleic acid technologies that may be implemented in a manner that specifically alters (e.g., increases or decreases in a statistically significant manner) expression of a herein disclosed TSC-derived polypeptide (e.g., one of SEQ ID NOS: 1-4, TSC peptide 17, 17A, 56, or 56B)-encoding polynucleotide, or of a polynucleotide that encodes a polypeptide as provided herein which comprises the TSC-derived polypeptide amino acid sequence, such as a polynucleotide that encodes any of the polypeptides having amino acid sequences set forth in SEQ ID NOS: 1-4 (TSC peptides 17, 17A, 56, 56B). Such antisense-based technologies include RNA interference, ribozymes and antisense nucleic acids.

RNA interference (RNAi) is a polynucleotide sequence-specific, post-transcriptional gene silencing mechanism effected by double-stranded RNA that results in degradation of a specific messenger RNA (mRNA), thereby reducing the expression of a desired target polypeptide encoded by the mRNA (see, e.g., WO 99/32619; WO 01/75164; U.S. Pat. No. 6,506,559; Fire et al., Nature 391:806-11 (1998); Sharp, Genes Dev. 13:139-41 (1999); Elbashir et al. Nature 411:494-98 (2001); Harborth et al., J. Cell Sci. 114:4557-65 (2001)). RNAi is mediated by double-stranded polynucleotides as also described herein below, for example, double-stranded RNA (dsRNA), having sequences that correspond to exonic sequences encoding portions of the polypeptides for which expression is compromised. RNAi reportedly is not effected by double-stranded RNA polynucleotides that share sequence identity with intronic or promoter sequences (Elbashir et al., 2001). RNAi pathways have been best characterized in Drosophila and Caenorhabditis elegans, but “small interfering RNA” (siRNA) polynucleotides that interfere with expression of specific polypeptides in higher eukaryotes such as mammals (including humans) have also been described (e.g., Tuschl, 2001 Chembiochem. 2:239-245; Sharp, 2001 Genes Dev. 15:485; Bernstein et al., 2001 RNA 7:1509; Zamore, 2002 Science 296:1265; Plasterk, 2002 Science 296:1263; Zamore 2001 Nat. Struct. Biol. 8:746; Matzke et al., 2001 Science 293:1080; Scadden et al., 2001 EMBO Rep. 2:1107) and subsequently elaborated upon.

According to a current non-limiting model, the RNAi pathway is initiated by ATP-dependent, processive cleavage of long dsRNA into double-stranded fragments of about 18-27 (e.g., 19, 20, 21, 22, 23, 24, 25, 26, etc.) nucleotide base pairs in length, called small interfering RNAs (siRNAs) (see review by Hutvagner et al., Curr. Opin. Gen. Dev. 12:225-32 (2002); Elbashir et al., 2001; Nyksnen et al., Cell 107:309-21 (2001); Zamore et al., Cell 101:25-33 (2000); Bass, Cell 101:235-38 (2000)). In Drosophila, an enzyme known as “Dicer” cleaves the longer double-stranded RNA into siRNAs; Dicer belongs to the RNase III family of dsRNA-specific endonucleases (WO 01/68836; Bernstein et al., Nature 409:363-66 (2001)). Further according to this non-limiting model, the siRNA duplexes are incorporated into a protein complex, followed by ATP-dependent unwinding of the siRNA, which then generates an active RNA-induced silencing complex (RISC) (WO 01/68836). The complex recognizes and cleaves a target RNA that is complementary to the guide strand of the siRNA, thus interfering with expression of a specific protein (Hutvagner et al., supra).

In C. elegans and Drosophila, RNAi may be mediated by long double-stranded RNA polynucleotides (WO 99/32619; WO 01/75164; Fire et al., 1998; Clemens et al., Proc. Natl. Acad. Sci. USA 97:6499-6503 (2000); Kisielow et al., Biochem. J. 363:1-5 (2002); see also WO 01/92513 (RNAi-mediated silencing in yeast)). In mammalian cells, however, transfection with long dsRNA polynucleotides (i.e., greater than 30 base pairs) leads to activation of a non-specific sequence response that globally blocks the initiation of protein synthesis and causes mRNA degradation (Bass, Nature 411:428-29 (2001)). Transfection of human and other mammalian cells with double-stranded RNAs of about 18-27 nucleotide base pairs in length interferes in a sequence-specific manner with expression of particular polypeptides encoded by messenger RNAs (mRNA) containing corresponding nucleotide sequences (WO 01/75164; Elbashir et al., 2001; Elbashir et al., Genes Dev. 15:188-200 (2001)); Harborth et al., J. Cell Sci. 114:4557-65 (2001); Carthew et al., Curr. Opin. Cell Biol. 13:244-48 (2001); Mailand et al., Nature Cell Biol. Advance Online Publication (Mar. 18, 2002); Mailand et al. 2002 Nature Cell Biol. 4:317).

siRNA polynucleotides may offer certain advantages over other polynucleotides known to the art for use in sequence-specific alteration or modulation of gene expression to yield altered levels of an encoded polypeptide product. These advantages include lower effective siRNA polynucleotide concentrations, enhanced siRNA polynucleotide stability, and shorter siRNA polynucleotide oligonucleotide lengths relative to such other polynucleotides (e.g., antisense, ribozyme or triplex polynucleotides).

By way of a brief background, “antisense” polynucleotides bind in a sequence-specific manner to target nucleic acids, such as mRNA or DNA, to prevent transcription of DNA or translation of the mRNA (see, e.g., U.S. Pat. Nos. 5,168,053; 5,190,931; 5,135,917; 5,087,617; see also, e.g., Clusel et al., 1993 Nucl. Acids Res. 21:3405-11, describing “dumbbell” antisense oligonucleotides). “Ribozyme” polynucleotides can be targeted to any RNA transcript and are capable of catalytically cleaving such transcripts, thus impairing translation of mRNA (see, e.g., U.S. Pat. Nos. 5,272,262; 5,144,019; and 5,168,053, 5,180,818, 5,116,742 and 5,093,246; U.S. 2002/193579). “Triplex” DNA molecules refers to single DNA strands that bind duplex DNA to form a colinear triplex molecule, thereby preventing transcription (see, e.g., U.S. Pat. No. 5,176,996, describing methods for making synthetic oligonucleotides that bind to target sites on duplex DNA). Such triple-stranded structures are unstable and form only transiently under physiological conditions.

Because single-stranded polynucleotides do not readily diffuse into cells and are therefore susceptible to nuclease digestion, development of single-stranded DNA for antisense or triplex technologies often requires chemically modified nucleotides to improve stability and absorption by cells. siRNAs, by contrast, are readily taken up by intact cells, are effective at interfering with the expression of specific polypeptides at concentrations that are several orders of magnitude lower than those required for either antisense or ribozyme polynucleotides, and do not require the use of chemically modified nucleotides.

It will be appreciated that the practice of the several embodiments of the present invention will employ, unless indicated specifically to the contrary, conventional methods in virology, immunology, microbiology, molecular biology and recombinant DNA techniques that are within the skill of the art, and many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

### Cit-TNC Oligopeptide Detection and Immunogenicity

The cit-TNC oligopeptides that contain epitopes recognized by and immunogenic for T-cells, as described herein (e.g., SEQ ID NOS:1-4 TNC peptides 17, 17A, 56, 56B, and variants thereof), may elicit a T-cell response. As used herein, a specific “T-cell response” refers to proliferation and activation of effector functions induced, in a statistically significant manner as will be understood by those skilled in the art, by a peptide in vitro or in vivo following contact with a specific peptide that can be recognized by a given T-cell via its expressed T-cell receptor for antigen (TCR), but not by a distinct control peptide having an irrelevant structure that is not recognized by the TCR. T-cell responses may be characterized according to any of a large number of art-accepted methodologies for assaying T-cell activity, including determination of T-cell activation or induction and also including determination of T-cell responses that are antigen-specific. Examples include determination of T-cell proliferation, T-cell cytokine release, antigen-specific T-cell stimulation, MHC-restricted T-cell stimulation, cytotoxic activity (e.g., by detecting ⁵¹Cr release from pre-loaded target T-cells and/or by caspase-3 assay (e.g., Jerome et al. 2003 Apoptosis 8:563; He et al., 2005 J. Imm. Meth. 304:43), changes in T-cell phenotypic marker expression (e.g., cell surface marker molecule expression as may be readily determined using antibodies that bind specifically to particular surface markers, as may be detected by flow immunocytofluorimetry (e.g., fluorescence activated cell sorting, FACS) or immunofluorescence microscopy, or other immunochemical techniques), and other measures of T-cell functions. Procedures for performing these and similar assays are may be found, for example, in Lefkovits (Immunology Methods Manual: The Comprehensive Sourcebook of Techniques, 1998). See also Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein).

Levels of cytokines may be determined according to methods described herein and practiced in the art, including for example, ELISA, ELISPOT, bead-based microplexing (e.g., Luminex® xMAP® systems), intracellular cytokine staining, flow cytometry, and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from synovial fluid, stimulating the cells with antigen, and measuring cytokine production, cell proliferation and/or cell viability, such as by enzyme-linked immunosorbent assays (ELISA) for released cytokines, or by quantifying cellular incorporation of tritiated thymidine to assess proliferation or by non-radioactive assays, such as MTT cellular viability assays, and the like. The effect of an immunogen described herein on a T-cell may be examined, for example, by determining levels of, e.g., IFNγ, IL-22, IL-10, IL-17, TGF-β, IL-35, IL-2, or other Treg cytokines and/or granzyme B expressed in or released by T-cells that have been exposed to a suitably presented cognate antigen under assay conditions with which those skilled in the art will be familiar.

The level of a T-cell immune response thus may be determined by any one of numerous immunological methods described herein and routinely practiced in the art. The level of a T-cell immune response may be determined prior to and following administration of any one of the herein described TNC-derived polypeptides (e.g., citrullinated oligopeptides) that contain epitopes recognized by, and immunogenic for, T-cells (or administration of a composition comprising a polynucleotide encoding such a polypeptide).

Antigen-specific T-cell immune responses are typically determined by comparisons of observed T-cell responses according to any of the herein described T-cell functional parameters (e.g., proliferation, cytokine release, cytotoxic/cytolytic T lymphocyte (CTL) activity, altered cell surface marker phenotype, etc.). Antigen-specific immunoreactivity may be detected by comparing (i) T-cells that are exposed to a cognate (e.g., TCR-recognizable) antigen in an appropriate context (e.g., the antigen used to prime or activate the T-cells, when presented by immunocompatible antigen-presenting cells, for example, antigen-presenting cells sharing at least one allelic form of an MHC gene that is relevant for antigen presentation) to (ii) T-cells from the same source population that are exposed instead to a structurally distinct or irrelevant control antigen (e.g., not TCR-recognizable). A response to the cognate antigen that is greater, with statistical significance, than the response to the control antigen signifies antigen-specificity. By similar criteria, antigen-specific binding of an antibody to a cognate antigen (e.g., one of the herein described cit-TNC-derived citrullinated oligopeptides) may be determined as a level of detectable binding that is greater, with statistical significance, than the level of binding of the antibody to a structurally irrelevant control antigen.

In certain embodiments, the T-cell response referenced herein is a CD4+ T-cell response. A non-limiting example of a CD4+ T-cell is a lymphocyte of the following phenotype CD4+CD14−CD19−. CD4+ T cell phenotypes associated with autoimmune disease are well characterized in the art (see e.g., Salmon M et al., 1995 Br Med Bull. 51:332; Fonseka C Y et al., 2017 Curr Opin Immunol. 49:27; Kondo Y et al., 2018 Arthritis Rheumatol. 70:653). In certain further embodiments, a CD4+ T-cell response may include a regulatory T-cell (Treg) response, which may include, by way of non-limiting example, an antigen-specific response by a CD4+CD14−CD19−T lymphocyte that expresses cell-surface CD25 and the transcription factor FOXP3. Treg phenotypes, including those associated with the modulation of autoimmune disease and arthritis have been previously documented (see e.g., van Amelsfort et al. 2004 Arthritis Rheum. 50:2775; Frey O et al., 2005 Arthritis Res Ther. 7:R291, PMID: 15743476; Esensten J H et al., 2009 Nat Rev Rheumatol. 5:560; Haque M et al. 2014 Front Oncol. 4:209).

Presently disclosed embodiments also contemplate compositions, including, for example, immunotherapeutic compositions, comprising one or more cit-TNC oligopeptides as provided herein, and a carrier. In certain embodiments an immunotherapeutic composition comprising at least one herein described citrullinated oligopeptide that is bound covalently or noncovalently to a carrier as provided herein, such as a solid carrier, may be prepared for infusion into a subject, for example and in certain embodiments, to promote immunological tolerance. In some instances, the carrier may be a nanoparticle or other solid carrier particle, including such carriers as described herein and/or in publications incorporated by reference, in which the cit-TNC oligopeptide is attached to the solid carrier covalently or non-covalently.

By way of non-limiting example, in certain contemplated embodiments an autoimmune response may be modulated (e.g., reduced in a statistically significant manner relative to an appropriate control) by administering to a subject having or suspected of being at risk for having a condition characterized by a tenascin C-specific autoimmune response, an immunotherapeutic composition comprising one or more herein disclosed cit-TNC oligopeptides and a carrier. Without wishing to be bound by theory, it is believed that modulation of an autoimmune response may be so achieved, for example, through the suppression of autoreactive T-cells and/or the induction of regulatory T cells (Tregs) and/or by another immunological mechanism in response to the administered immunotherapeutic composition. Methods for induction of antigen-specific immunological tolerance using carrier particle-borne tolerizing antigens are documented in the art (e.g., Serra et al., 2018 Eur J Immunol. 48:751; Getts et al., 2015 Trends Immunol. 36:419; Clemente-Casares et al., 2016 Nature 530: 434-40; Getts et al., 2012 Nat. Biotechnol. 30: 1217-24; Hunter et al., 2014 ACS Nano. 8: 2148-60; Kim et al., 2002 Arthritis Rheum. 46: 1109-20; Bryant et al., 2014 Biomaterials 35: 8887-94; Pearson et al., 2017 Mol. Ther. 25: 1655-64). These and related methods as will be known to those familiar with the art may be modified according to the teachings herein for use with the presently disclosed cit-TNC peptides (e.g., the citrullinated oligopeptide comprising any of SEQ ID NOS:1-4, TNC peptides 17, 17A, 56, 56B) to induce antigen-specific immune tolerance and reduce autoimmune response levels. By way of non-limiting theory, such antigen-specific suppression of inappropriate immunological reactivity against citTNC is contemplated to provide unprecedented amelioration of autoimmune disease in RA patients.

Immunotherapeutic compositions comprising a carrier and cit-TNC oligopeptides may comprise one or more citrullinated oligopeptide having the amino acid sequence of any of SEQ ID NOS: 1-4 (TSC peptides 17, 17A, 56, or 56B, or any of the amino acid sequences shown in FIG. 2, 5, 9, 12, or 14 or Tables 1 or 2. The carrier may be a solid carrier and is preferably a biocompatible solid carrier (e.g., Tostanoski et al., 2016 Discov. Med. 21:403; Hunter et al., 2014 ACS Nano. 8:2148; Prasad et al., 2012 Rev. Diabet. Stud. 9:319), which may, in some embodiments, comprise a nanoparticle (e.g., a particle having no dimension that exceeds 1 to 200 μm in size), a microparticle (e.g., 1 to 1000 μm in size), a macroparticle (e.g., 2500 to 10,000 nm), or a magnetic bead. Examples of such solid carriers include, but are not limited to particles composed of metals (gold, copper oxide, aluminum oxide, zinc oxide, iron oxide, and silver) and polymers (chitosan, polylactic acid (PLA), poly[lactic-co-glycolic]acid (PLGA)). One or more peptides can be bound to the solid carriers by covalent or non-covalent chemical interactions according to any of a variety of established methodologies such as those described in the cited literature. Covalent chemical interactions may, for example, involve the conjugation of a reactive moiety on the C-terminus of a peptide to a reactive moiety on the surface of the solid carrier. Non-covalent chemical interactions may include, but are not limited to, affinity interactions (e.g., between avidin and biotin, between antigen and antibody, and/or between receptor and ligand), ionic interactions, and/or hydrophobic interactions. Methods of covalently and non-covalently binding a peptide to a solid carrier are known in the art and have been described previously (e.g., U.S. Pat. No. 8,895,023; US 2020/0095546; US 2013/0337005; US 2006/0233712).

Certain other presently disclosed embodiments, by way of illustration and not limitation, contemplate methods for detecting presence in a biological sample of an antibody that specifically binds to citrullinated tenascin-C. These and related embodiments may be useful for characterizing a biological sample, such as a biological sample obtained from a subject having or suspected of having one or more antibodies that specifically bind to human tenascin-C, for instance, a subject having or suspected of having rheumatoid arthritis, as may be selected according to previously designated criteria (e.g., Wasserman et al., 2011 Am. Fam. Physician 84:1245; Smolen et al., Nature Rev. Dis. Primers 4:18001; Kay et al., 2012 Rheumatol. 51 Suppl. 6:vi5-9). As provided herein, a cit-TNC-specific antibody may be detected by contacting a biological sample with a cit-TNC peptide, and quantitatively determining a test level of specific binding of an antibody in the sample to the cit-TNC peptide (e.g., a citrullinated oligopeptide comprising any of the presently disclosed cit-TNC peptides 17, 17A, 56, or 56B (SEQ ID NOS: 1-4) or any of the amino acid sequences shown in FIG. 2, 5, 9, 12, or 14 or Tables 1 or 2) as compared to the level of binding of an antibody in the sample to a control non-citrullinated oligopeptide, to assess presence in the biological sample of an antibody that specifically binds to citrullinated human tenascin-C.

Exemplary methods for the detection of anti-citrullinated peptide/protein antibodies (ACPA) in biological samples have been previously described (e.g., Trier et al., 2018 J Immunol Methods. 454:6; Dubois-Galopin et al., 2006 Ann Biol Clin (Paris) 64:162; Aggarwal et al., 2009 Arthritis Rheum. 61:1472) such that adaptation of these methodologies to the presently disclosed embodiments is contemplated for detection of cit-TNC-specific autoantibodies, based on the teachings provided herein, including the presently disclosed cit-TNC peptides 17, 17A, 56, or 56B (SEQ ID NOS: 1-4), or any of the amino acid sequences shown in FIG. 2, 5, 9, 12, or 14 or Tables 1 or 2. In certain related embodiments disclosed herein, as also discussed below, there is also contemplated a method for the isolation of TNC-specific antibodies comprising, in pertinent part, incubating a biological sample suspected of containing such antibodies with a herein described citrullinated TNC oligopeptide under conditions permissive for formation of antibody-antigen immune complexes, removing unbound antibodies and recovering the immune complexes, and therefrom isolating TNC-specific antibodies.

An immunoassay substrate composition comprising cit-TNC oligopeptides (e.g., the presently disclosed cit-TNC peptides 17, 17A, 56, or 56B (SEQ ID NOS: 1-4), or any of the amino acid sequences shown in FIG. 2, 5, 9, 12, or 14 or Tables 1 or 2) and a solid carrier is also contemplated herein according to certain embodiments, for instance, for use in detecting the presence of antibodies specific to a TNC-derived peptide to assist in the diagnosis of rheumatoid arthritis or other disease associated with the overabundance of TNC-derived peptides. As described herein, one or more peptides may be bound to the solid carrier by covalent or non-covalent chemical interactions. The composition so formed may be used as an immunoassay substrate in which the solid carrier may be a tube, an assay plate, a well of a multi-well plate, a membrane, a nanoparticle, microparticle, macroparticle, magnetic bead, and/or any other suitable solid phase on which a test antigen can be immobilized. In some embodiments, the solid carrier may be a nanoparticle (e.g., a particle having no dimension in excess of 1 to 200 μm in size), a microparticle (e.g., 1 to 1000 μm in size), a macroparticle (e.g., 2500 to 10,000), or a magnetic bead. Examples of such solid carriers include, but are not limited to particles composed of metals (gold, copper oxide, aluminum oxide, zinc oxide, iron oxide, and silver) and polymers (chitosan, polylactic acid (PLA), poly[lactic-co-glycolic] acid (PLGA)). The immunoassay substrate composition may be used for detecting in a biological sample the presence and/or quantity of citrullinated human tenascin C peptide-specific antibodies that bind specifically to at least one herein described cit-TNC oligopeptide, according to any of the antibody-detecting immunoassayformats described herein or otherwise known generally in the art.

Immune complexes can be detected and quantified using a number of methods known in the art, including but not limited to, immune-enzymatic processes comprising enzyme-linked immunosorbent assays (ELISA), indirect immunofluorescense assays (IFA), radioimmunological assays (RIA), lateral flow immunoassays, immunoblotting, immunoaffinity chromatography, immunoprecipitation, and flow cytometry (see e.g., US2016/0377620; U.S. Pat. No. 9,470,682; US2019/0277857; US2020/0166505; Jason et al. J. Rheum. 31:1). Following a step of incubating a reaction mixture that is formed by contacting a biological sample suspected of containing antibodies that specifically bind TNC with at least one of the herein described citrullinated TNC oligopeptides under conditions permissive for immune complex formation between the antibodies and the oligopeptides, detection and/or quantification of a cit-TNC-specific antibody may, in some embodiments, include isolating one or more immune complexes from the reaction mixture. For instance, this step may involve first removing from the reaction mixture antibodies from the biological sample that are not specifically bound to the citrullinated oligopeptide, followed by recovering from the reaction mixture said immune complexes, and thereby isolating tenascin-C-specific antibodies. A wide range of immunochemical methodologies are known for separating immune complexes from unbound antibodies. Certain further embodiments may comprise quantifying the recovered immune complexes that have formed by citrullination-dependent binding, for example, by determining a test level of specific binding of one or more antibodies to a cit-TNC oligopeptide relative to a control level of specific binding to a non-citrullinated polypeptide comprising the correspondingTNC oligopeptide(s) of general formula (I) in which there is no citrullinated arginine residue.

A binding partner or an antibody is said to be “immunospecific,” “specific for” or to “specifically bind” an immunogen of interest if the antibody reacts at a detectable level with the immunogen or immunogenic fragment thereof, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{d}, and an antibody specifically binds to the immunogen of interest if it binds with a K_{d} of less than or equal to 10⁻⁴ M, less than or equal to about 10⁻⁵ M, less than or equal to about 10⁻⁶ M, less than or equal to 10⁻⁷ M, or less than or equal to 10⁻⁸ M.

Affinities of binding partners or antibodies can be readily determined using conventional techniques, for example, those described by Scatchard et al. (Ann. N. Y. Acad. Sci. USA 51:660 (1949)), or by surface plasmon resonance (SPR; BIACORE™, Biosensor, Piscataway, N.J.), or according to other procedures described in literature cited herein or otherwise known in the art. For surface plasmon resonance, by way of non-limiting example, target molecules are immobilized on a solid phase surface and exposed to a binding partner (or ligand) in a mobile phase running along a flow cell in a light sensor. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al., Cancer Res. 53:2560-2565 (1993)).

According to certain embodiments disclosed herein, a biological sample may be obtained from a subject, for determining in the sample the presence and/or level of an immune response that is specific for an immunogenic TNC-derived polypeptide as described herein. In certain preferred embodiments, a specific immune response is detected (e.g., antibodies or T-cell reactivity) against any of the presently disclosed cit-TNC peptides 17, 17A, 56, or 56B (SEQ ID NOS: 1-4), or any of the amino acid sequences shown in FIG. 2, 5, 9, 12, or 14 or Tables 1 or 2, that contains an epitope recognized by, and immunogenic for, T-cells and B-cells. A “biological sample” as used herein for these and other embodiments may be a blood sample (e.g., blood, from which serum or plasma may be prepared), another body fluid (e.g., lymph, synovial fluid, saliva, sputum, bronchoalveolar lavage (BAL) fluid, urine, cerebrospinal fluid (CSF), serosal fluid), a stool sample, or any other tissue or cell preparation from the subject or a biological source. Biological samples may also be obtained from a subject prior to the subject having been administered any immunogenic composition, which biological sample may be useful in certain embodiments as a control for establishing baseline (i.e., pre-treatment) TNC-specific (including cit-TNC-specific) immunoreactivity data.

In another embodiment there is provided an antigen-specific immunomodulatory composition comprising any one or more of the presently disclosed cit-TNC oligopeptides 17, 17A, 56, or 56B (SEQ ID NOS: 1-4), or any of the cit-TNC amino acid sequences shown in FIG. 2, 5, 9, 12, or 14 or Tables 1 or 2, and a carrier capable of inducing tenascin C-specific immunological tolerance. By way of illustration and not limitation, the antigen-specific immunomodulatory composition may induce tenascin C-specific immunological tolerance that is MHC class II molecule-restricted, wherein the MHC class II molecule is HLA-DRB1*04:01. In some embodiments, the carrier may be a solid carrier, which, as described herein, may include a nanoparticle in which one or more peptides are bound to the solid carriers by covalent or non-covalent chemical interactions.

Also contemplated, by way of illustration and not limitation, are immunotherapeutic protocols involving the adoptive transfer to a subject (e.g., an RA patient) of antigen-presenting cells that have been pulsed in vitro with one or more of the herein disclosed immunogenic cit-TNC oligopeptides or with cit-TNC protein, and/or adoptive transfer to the subject of immunocompatible cit-TNC-specific T-cells that have been induced in vitro by exposure to such antigen-presenting cells (APC) that have been pulsed in vitro with immunogenic cit-TNC peptides such that the APC present the immunogenic cit-TNC peptides to the T-cells at the APC cell surface.

In certain related embodiments, the adoptively transferred cit-TNC-specific T-cells are CD4+ T cells or Treg cells. In certain further embodiments, the T-cell responds antigen-specifically to a citrullinated TNC oligopeptide in a MHC class II molecule-restricted manner, which MHC class II molecule in certain still further embodiments is HLA-DRB1*04:01. Principles of antigen processing by antigen-presenting cells (APC) such as dendritic cells, macrophages, lymphocytes and other cell types, and of antigen presentation by APC to T-cells, including major histocompatibility complex-(MHC) restricted presentation between immunocompatible APC and T-cells, are well established (see, e.g., Murphy, Janeway's Immunobiology (8th Ed.) 2011 Garland Science, NY; chapters 6, 9 and 16). Adoptive transfer protocols using unselected or selected T-cells are also known in the art (e.g., US2011/0052530, US2010/0310534; Ho et al., 2006 J. Imm. Meth. 310:40; Ho et al., 2003 Canc. Cell 3:431) and may be modified according to the teachings herein for use with transfer cell populations containing T-cells that are specifically induced by one or more of the presently disclosed immunogenic cit-TNC-derived T-cell epitope-containing oligopeptides. Adoptive transfer to the subject of cit-TNC-specific T-cells described herein may, in some embodiments, be preceded by an in vitro T-cell expansion step. Following in vitro induction of cit-TNC-specific T-cells by exposure to antigen-presenting cells, the resulting mixture may be cultured for a period of time sufficient to generate adequate cell numbers (see e.g., Tang et al., 2012 Curr Opin Organ Transplant. 17:349) for adoptive transfer.

In certain embodiments, the antigen-specific T cell response is a CD4+ T cell response, and in certain embodiments, the T cell response is a Treg cell response. T cell expansion protocols are well known in the art such that adaptation of these methodologies to the presently disclosed embodiments is contemplated, based on the teachings herein, including those that are directed to the expansion of Tregs (see e.g., Hoffmann et al., 2004 Blood 104:895; Hippen K L et al., 2011 Sci Transl Med. 3:83ra41, PMID:21593401; Fraser et al., 2018 Mol Ther Methods Clin Dev. 8:198; Wiesinger et al., 2017 Front Immunol. 8:1371). Gene editing strategies to obtain cells exhibiting a Treg phenotype by induced FoxP3 expression in T cells are described in WO/2018/080541 and in WO/2019/210078. Exemplary details of forced FOXP3 expression by gene editing including knock-in (insertion) of a full length, codon-optimized FoxP3 cDNA into the FOXP3 or AAVS1 locus may be found in WO/2019/210042. Certain methods for phenotypic and functional characterization of Treg cells, including but not limited to cells in which FoxP3 overexpression has been induced, are known in the art (e.g., WO/2018/080541, WO/2019/210078, McMurchy et al., 2013 Meths. Mol. Biol. 946: 115-132; Thornton et al., 2019 Eur. J. Immunol. 49:398-412; Aarts-Riemens et al., 2008 Eur. J. Immunol. 38: 1381-1390; McGovern et al., 2017 Front. Immunol. 8: Art. 1517.

According to certain preferred embodiments the Treg response is antigen-specific and results from antigen-specific recognition by a Treg cell of at least one of the presently disclosed cit-TNC oligopeptides 17, 17A, 56, or 56B (SEQ ID NOS: 1-4), or of at least one of the cit-TNC amino acid sequences shown in FIG. 2, 5, 9, 12, or 14 or Tables 1 or 2. Without wishing to be bound by theory, such Treg recognition of an epitope present in the citrullinated oligopeptide is mediated by a TCR expressed by the Treg cell, which TCR may in certain embodiments be the expressed product of TCR alpha- and/or beta-chain genes native to the Treg cell. Preferably such antigen-specific recognition depends on recognition of an epitope that comprises a citrullinated arginine residue.

In certain other embodiments, however, a T-cell population may be antigenically stimulated in vitro with the herein disclosed cit-TNC oligopeptides and clonally expanded in vitro to provide clonal T cells from which the TCR alpha- and/or beta-chain genes may be isolated, introduced into the Treg cell by genome engineering (e.g., CRISPR/Cas-mediated gene editing or equivalent) or by other transduction or transfection methods, and functionally expressed to provide a population of engineered cit-TNC-specific Treg cells suitable for adoptive transfer immunotherapy into, e.g., a rheumatoid arthritis patient. In related further embodiments, the cit-TNC antigen-specific T cell response is MHC class II molecule-restricted, and in certain still further embodiments the MHC class II molecule may be HLA-DRB1*04:01.

More specifically, certain presently disclosed embodiments thus contemplate, for example, clonally expanding cit-TNC-reactive T-cells that have been induced in vitro by exposure to antigen-presenting cells that have been pulsed in vitro with the presently disclosed immunogenic cit-TNC peptides (e.g., SEQ ID NOS: 1-4, cit-TNC peptides 17, 17A, 56, 56B), and from such T-cells identifying and cloning the functional (e.g., productively rearranged) T-cell receptor (TCR)-encoding genes, which may then be used to transfect/transduce a T-cell population to generate engineered tenascin C-specific T-cells for adoptive transfer into subjects. In certain embodiments, the T-cell receptor (TCR)-encoding genes are isolated from either a CD4+ T cell or a Treg cell. Recent advances in TCR sequencing have been described (e.g., Robins et al., 2009 Blood 114:4099; Robins et al., 2010 Sci. Translat. Med. 2:47ra64, PMID: 20811043; Robins et al. 2011 J. Imm. Meth. 375:14; Warren et al., 2011 Genome Res. 21:790) and may be employed in the course of practicing these embodiments according to the present disclosure. Similarly, methods for transfecting/transducing T-cells with desired nucleic acids have been described (e.g., US2004/0087025) as have adoptive transfer procedures using T-cells of desired antigen specificity (e.g., Schmitt et al., 2009 Hum. Gen. 20: 1240; Dossett et al., 2009 Mol. Ther. 17:742; Till et al., 2008 Blood 112:2261; Wang et al., 2007 Hum. Gene Ther. 18:712; Kuball et al., 2007 Blood 109:2331; US2011/0243972; US2011/0189141; Leen et al., 2007 Ann. Rev. Immunol. 25:243), such that adaptation of these methodologies to the presently disclosed embodiments is contemplated, based on the teachings herein, including those that are directed to specific cit-TNC-derived oligopeptides that are capable of eliciting antigen-specific T-cell responses.

T-cells of desired antigen specificity may, in certain embodiments, be generated using nucleic acids (e.g., TCR-encoding nucleic acids) contained in an expression vector, or in a genomically targeted DNA repair template for use with a targeted gene editing DNA endonuclease (e.g., CRISPR/Cas or equivalent). One of skill in the art can readily ascertain suitable vectors or DNA repair template constructs comprising TCR-encoding nucleic acid sequences, for use with certain herein disclosed embodiments. A typical vector may comprise a nucleic acid molecule capable of transporting another nucleic acid to which it has been linked, or which is capable of replication in a host organism. Some examples of vectors include plasmids, viral vectors, cosmids, and others. Some vectors may be capable of autonomous replication in a host cell into which they are introduced (e.g., bacterial vectors having a bacterial origin of replication and episomal mammalian vectors), whereas other vectors may be integrated into the genome of a host cell upon introduction into the host cell and thereby replicate along with the host genome. Additionally, some vectors are capable of directing the expression of genes to which they are operatively linked (these vectors may be referred to as “expression vectors”.)

In certain other embodiments, engineered cit-tenascin C-specific T-cells may be artificially induced to express a Treg phenotype prior to adoptive transfer to a subject in need of treatment for RA or another condition characterized by a tenascin C-specific autoimmune response. A number of protocols for artificially generating Tregs are known in the art, including ex vivo and in vivo expansion systems containing exogenous factors such as rapamycin, IL-2, or TGF-β (Putnam et al., 2009 Diabetes 58:652; Battaglia et al. 2006 J Immunol. 177:8338; Monti et al., 2008 Diabetes 57:2341; Matsuoka et al., 2013 Sci Transl Med. 5:179ra43; Koreth et al. 2011 N Engl J Med. 365:2055; Yu et al. 2015 Diabetes 64:2172; US2020/0056152; Zheng et al., 2008 J Immunol 180:7112). Systems utilizing transgenes for the constitutive (see e.g. Allan et al. 2008 Mol Ther. 16:194; US2019/0247443; Seng et al. 2020 Blood Adv 4:1325) and inducible (see e.g. US2019/0247443; US2010/0203068; Allan et al., 2008 Mol Ther. 16:194) expression of FOXP3 in T-cells have also been described and may be employed in the course of practicing these embodiments according to the present disclosure.

Production of Treg cells for therapeutic uses is thus generally known in the art and may be accomplished by any of a number of known methodologies, for example by way of illustration and not limitation, isolation of nTreg from peripheral blood with in vitro expansion, isolation of Tr cells (e.g., IL-10 producing), induction of Treg using TGFb, +/−IL-1b or using other chemical means (e.g., Vitamin D), induced expression of FOXP3 following lentivirus (LV) transduction, induced FOXP3 expression following gene editing, or induced IL-10 expression (e.g., via lentiviral transduction or gene editing) to create TR1 cells. See, e.g., Raffin et al., 2020 Nat. Rev. Immunol. 20: 158-72; Roncarolo et al., 2018 Immunity 49: 1004-1009; Kanamori et al., 2016 Trends Immunol. 37: 803-811; Schneider and Buckner, 2011 Methods Mol. Biol. 707: 233-41. As provided herein, such Treg cells may then be engineered to express cit-TNC-specific TCR to obtain antigen-specific T cells that can respond specifically to the presently disclosed cit-TNC oligopeptides.

Methods for detecting human tenascin C-specific T cell-mediated immune response activity in a subject in need of treatment for RA, or for another condition characterized by a tenascin C-specific autoimmune response, are also presently contemplated. The method may encompass in vitro detection and functional characterization in a biological sample of T-cells that specifically bind to a cit-TNC oligopeptide as herein disclosed, such as a cit-TNC polypeptide comprising one or more of the presently disclosed cit-TNC oligopeptides 17, 17A, 56, or 56B (SEQ ID NOS: 1-4), or any of the cit-TNC amino acid sequences shown in FIG. 2, 5, 9, 12, or 14 or Tables 1 or 2, or the herein disclosed isolated citrullinated oligopeptide of no more than 18, 17, 16, 15, 14, 13, 12, 11, 10, or 9 amino acids, preferably of no more than 14, 13, 12, 11, 10, or 9 amino acids, which comprises a polypeptide of general formula (I) which comprises at least one arginine residue (R) that is citrullinated ([Cit]):

N—X—C (I)

said citrullinated oligopeptide being capable of eliciting an antigen-specific T-cell response to human tenascin-C specific or specifically binding an antibody, wherein [R/Cit] is either arginine or citrullinated arginine and preferably is citrullinated arginine, and wherein,
(a) X comprises an amino acid sequence that is selected from:

| | [Peptide 17A] |
|---|---|
| (SEQ ID NO: 1) | |
| | IS[R/Cit][R/Cit]GDMSS |
| | (TNC 874-882/Cit 876-877), |
| | [Peptide 17] |
| (SEQ ID NO: 2) | |
| | SLIS[R/Cit][R/Cit]GDMSSNPA |
| | (TNC 872-885/Cit 876-877), |
| | [Peptide 56] |
| (SEQ ID NO: 3) | |
| | GQYEL[R/Cit]VDL[R/Cit]DHGE |
| | (TNC 2068-2081/Cit2073, 2077), |
| | and |
| | [Peptide56B] |
| (SEQ ID NO: 4) | |
| | YEL[R/Cit]VDL[R/Cit]D |
| | (TNC 2070-2078/Cit 2073, 2077); |

(b) N is an amino terminus of the oligopeptide and consists of 0, 1, 2, or 3 amino acids that are independently selected from natural amino acids and non-natural amino acids; and
(c) C is a carboxy terminus of the oligopeptide and consists of 0, 1, 2, or 3, amino acids that are independently selected from natural amino acids and non-natural amino acids. Preferably and in certain embodiments, in general formula [I] N—X—C, the amino acid sequence of X in general formula [I] is citrullinated at two arginine residues.

Steps for detection and quantification may include contacting a biological sample with a cit-TNC peptide and quantitatively determining levels of T-cells that specifically bind citrullinated tenascin-C oligopeptides as compared to a suitable control peptide of unrelated structure as will be known to the skilled artisan (e.g., a “scrambled” peptide having a similar overall distribution of charged, neutral, and/or hydrophobic amino acid side chains but a distinct sequence), to assess presence in the biological sample of T cells that specifically bind to citrullinated human tenascin-C. Methods for the detection of antigen-specific T cells have been previously described; non-limiting examples include use of fluorescently-labelled MHC-Multimers combined with flow cytometry, cell surface trapping of cytokines using magnetic bead technology (e.g. Miltenyi cytokine-capture System®), CD25/OX40 co-expression assays, and enhanced flow cytometry techniques for single-cell analysis (see e.g. Bentzen et al., 2017 Cancer Immunol Immunother. 66: 657; Manz et al., 1995 Proc Natl Acad Sci USA 92:1921; Zaunders et al., 2009 J Immunol. 183:2827; US2017/0343545; US2019/0094224; U.S. Pat. No. 10,202,640).

In certain additional embodiments, functional characterization of a human tenascin C-specific T cell-mediated immune response is also currently contemplated. Steps may include contacting a biological sample comprising T lymphocytes (T cells) from a subject (e.g., a subject having or suspected of being at risk for RA) with a cit-TNC peptide under conditions (e.g., including appropriate presentation to T-cells by immunocompatible antigen-presenting cells) and for a time sufficient for specific cit-TSC T-cell epitope-containing oligopeptide recognition by one or more T cell receptors (TCR) and stimulation of a T-cell response. A “T-cell response” refers, e.g., to antigen-specifically induced T-cell proliferation and/or activation of T-cell effector functions as induced by an antigenic peptide in vitro or in vivo following contact with a specific peptide antigen, and may be characterized according to any of a large number of art accepted methodologies for assaying T-cell activity, for example, those described elsewhere herein.

In certain embodiments, the T cell to be characterized is a Treg cell, and Treg cell activity further includes Treg cell proliferation, Treg suppression of antigen-stimulated T cell proliferation, Treg suppression of antigen-independent T cell proliferation, and/or release of at least one Treg cytokine. Assays for evaluating Treg suppression and T cell proliferation have been described previously and may be adapted for use in the currently disclosed embodiments (see e.g. Collison et al. 2011 Methods Mol Biol. 707:21.). Levels of cytokines may also be determined according to methods described elsewhere herein and generally known in the art. As non-limiting examples, T cell-derived cytokines, factors, and mediators that may be detected as indicators of T cell immune response activity include IFNγ, IL-22, IL-10, IL-17, TGF-β, IL-35, IL-2, and/or granzyme B. Functional characterization of T cells by these or other indicator criteria may also be combined with techniques for detecting the presence of antigen-specific T cells (e.g., detection of cell surface-bound fluorescently-labelled MHC-multimers by flow cytometry) as an approach for targeted characterization of tenascin C-specific T cells.

The level of a T-cell immune response thus may be determined by any one of numerous immunological methods described herein and/or routinely practiced in the art. The level of a T-cell immune response may be determined prior to and following administration of any one of the herein described TNC-derived cit-TNC polypeptides or oligopeptides that contain epitopes recognized by, and immunogenic for, cit-TNC-specific T-cells (or in certain alternative embodiments, following administration of a composition comprising a polynucleotide encoding such a polypeptide).

As used in this specification and the appended claims, the singular forms “a,” “an” and “the” include plural references unless the content clearly dictates otherwise. Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Each embodiment in this specification is to be applied mutatis mutandis to every other embodiment unless expressly stated otherwise.

### EXAMPLES

### Example 1

### Citrullinated-Tenascin-C Peptides Bind HLA-DRB1*04:01 and are Immunogenic.

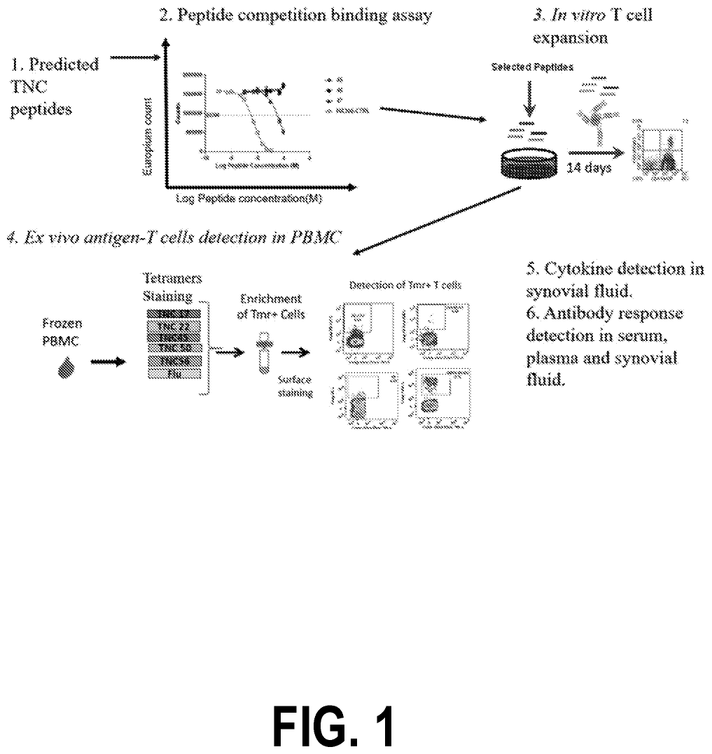

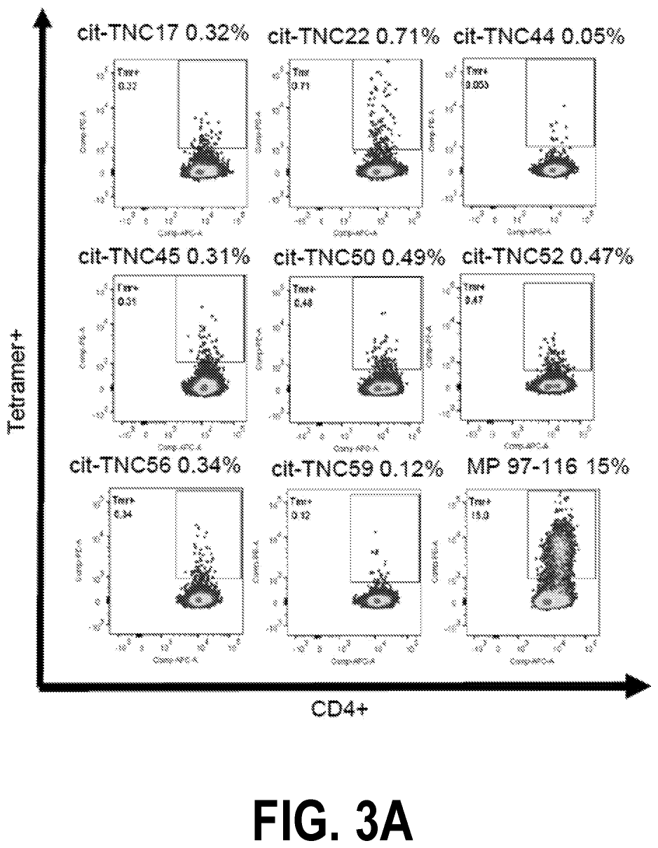

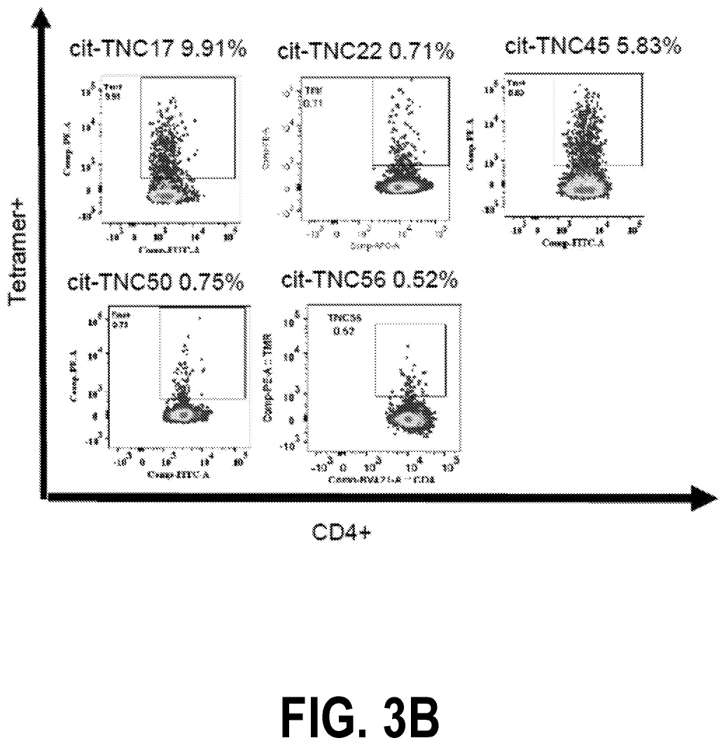

TNC is a large protein (2,110 amino acids in length) comprised of an N-terminal assembly domain, 14.5 epidermal growth-factor-like repeats (EGF-L), 8 constant fibronectin type III-like repeats (FNIII) plus an additional 9 alternatively spliced FNIII elements, and a C-terminal fibrinogen-like globe (FBG) (Midwood and Orend 2009 J Cell Commun Signal. 3:287). A previously described algorithm (James et al. 2014 Arthritis Rheumatol. 66:1712; James et al. 2010 Arthritis Rheum. 62:2909), was used to scan the entire length of the TNC monomer. Sixty-four arginine-containing peptides were identified with motifs that would be predicted to permit binding to HLA-DRB1*04:01 in either their non-citrullinated native form or citrullinated form or both. Based on these predictions, citrullinated versions of these peptides were synthesized and tested for their ability to bind HLA-DRB1*04:01 and to elicit CD4+ T cell expansion in vitro (FIG. 1). Of the peptides synthesized, 8 cit-TNC peptides bound to HLA-DRB1*04:01 with moderate to high affinity (FIG. 2). Among these, cit-TNC17, cit-TNC22, cit-TNC45, cit-TNC50 and cit-TNC56 were determined to be the most immunogenic, based on their ability to drive epitope-specific T cell expansion, as measured by HLA Class II tetramer staining of peripheral blood mononuclear cells (PBMC) from HLA-DRB1*04:01 positive subjects following two weeks of in vitro expansion (FIG. 2 and FIG. 3A-3B).

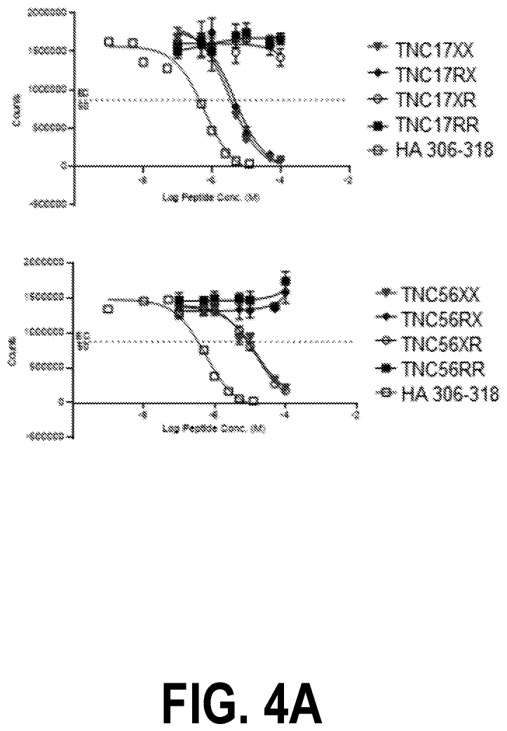

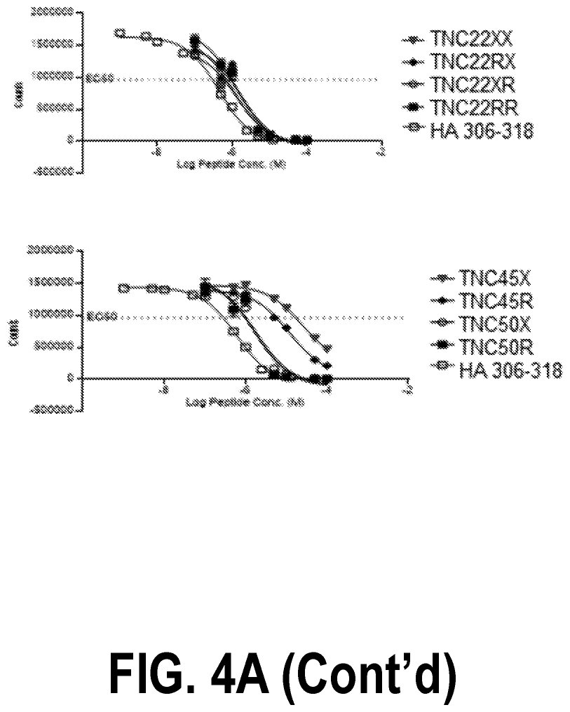

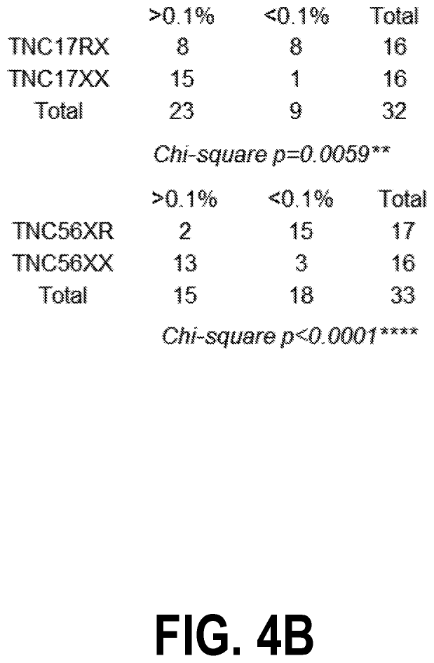

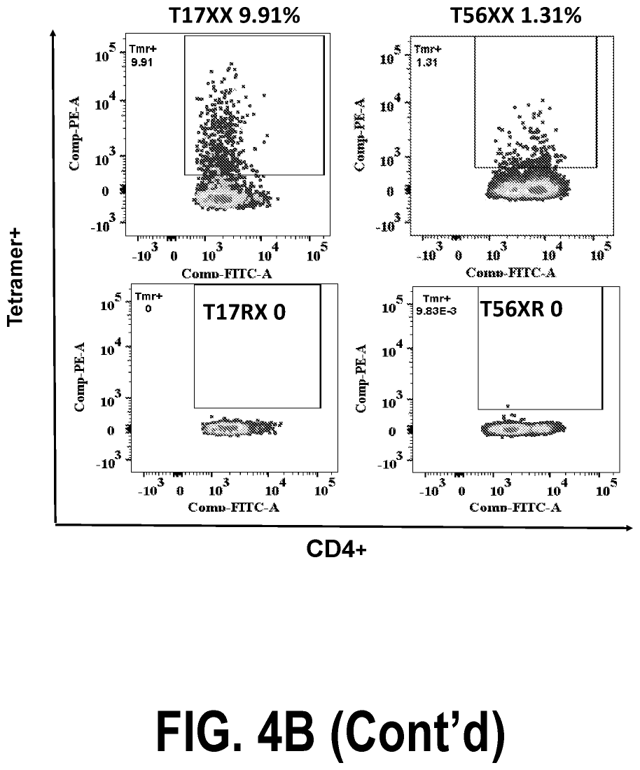

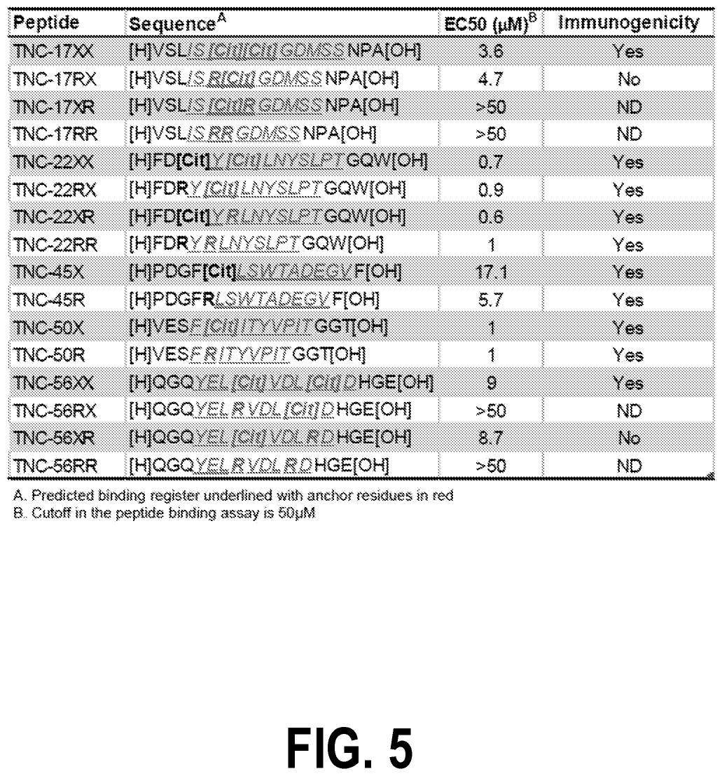

To assess whether citrullination was required for the HLA binding and immunogenicity of these peptides, the corresponding unmodified arginine-containing peptides (arg-TNC) were synthesized. Among the arg-TNC peptides synthesized, arg-TNC22, arg-TNC45 and arg-TNC50 bound to HLA-DRB1*04:01, whereas arg-TNC17 and arg-TNC56 did not (FIG. 4A and FIG. 5). This result suggested that cit-TNC17 and cit-TNC56 were citrullinated at sites that were critical for binding. Indeed, the cit-TNC-17 and cit-TNC56 peptides each contained a citrulline residue that was predicted to bind in pocket 4 of HLA-DRB1*04:01. To determine whether any of the citrullinated sites were critical for TCR recognition, the immunogenicity of the arg-TNC22, arg-TNC45 and arg-TNC50 were assessed, observing that each of these non-citrullinated versions had the ability to elicit T cell responses in vitro (FIG. 5). Partially citrullinated versions of cit-TNC17 and cit-TNC56, were also investigated, showing that each contained a predicted TCR contact that was associated with increased immunogenicity (FIG. 4B). These findings indicated that TNC had the ability to promote T cell responses in both its citrullinated and non-citrullinated forms but that some epitopes were uniquely immunogenic when citrullinated.

### Example 2

### The Frequency of Cit-Tenascin-C-Specific Cd4+ Memory T-Cells is Increased in RA Subjects

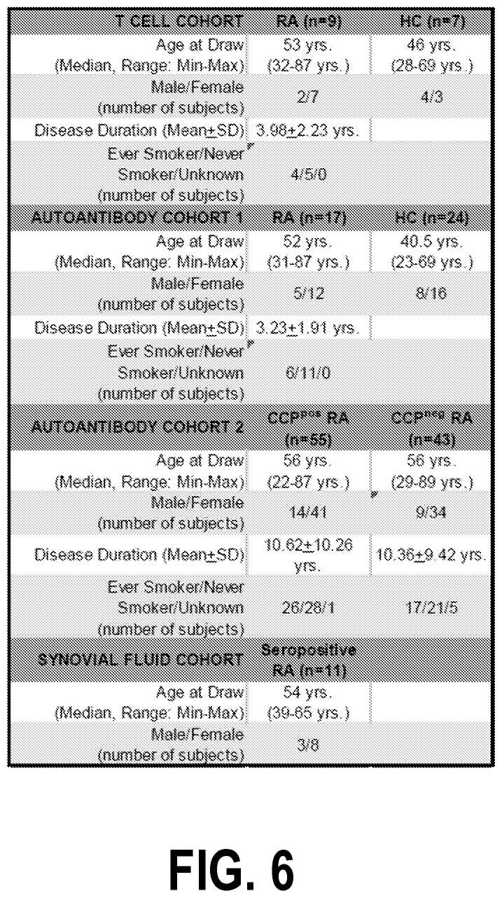

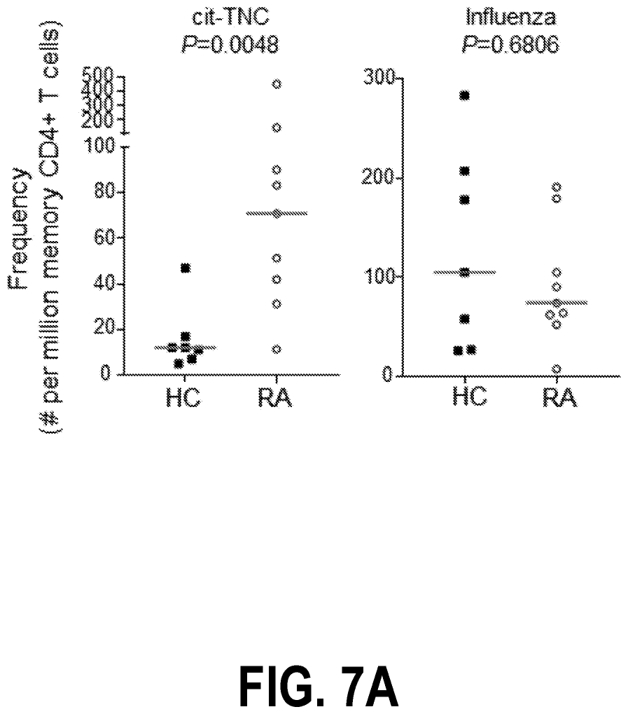

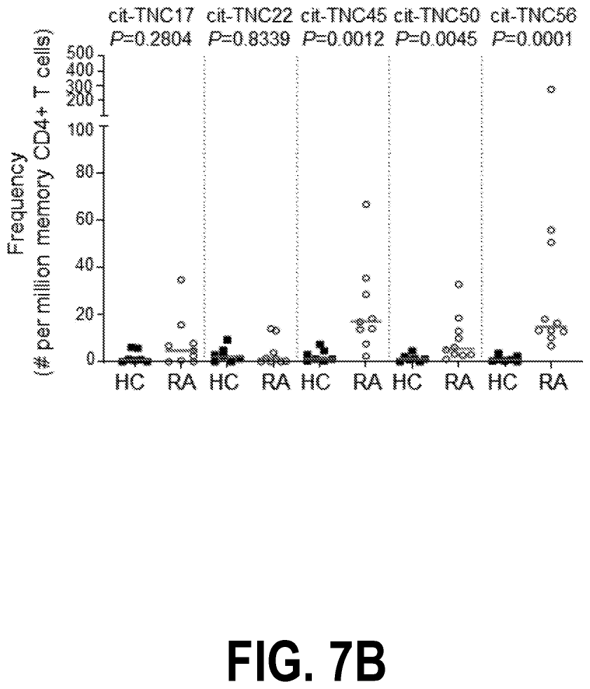

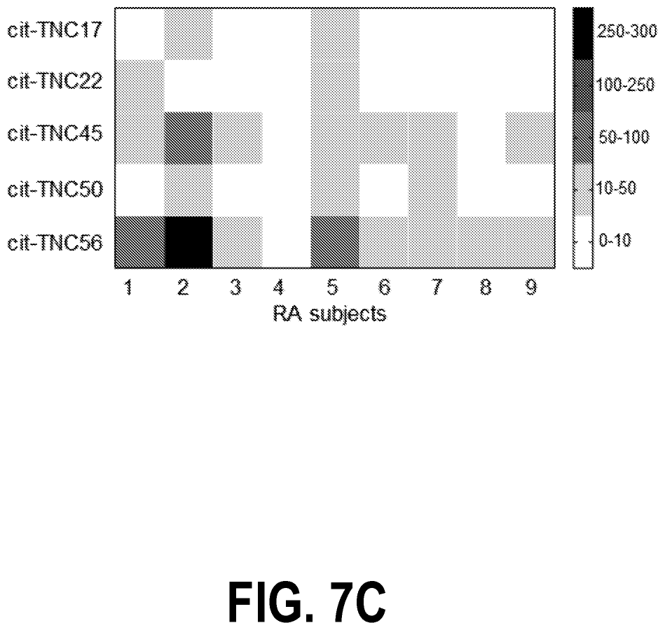

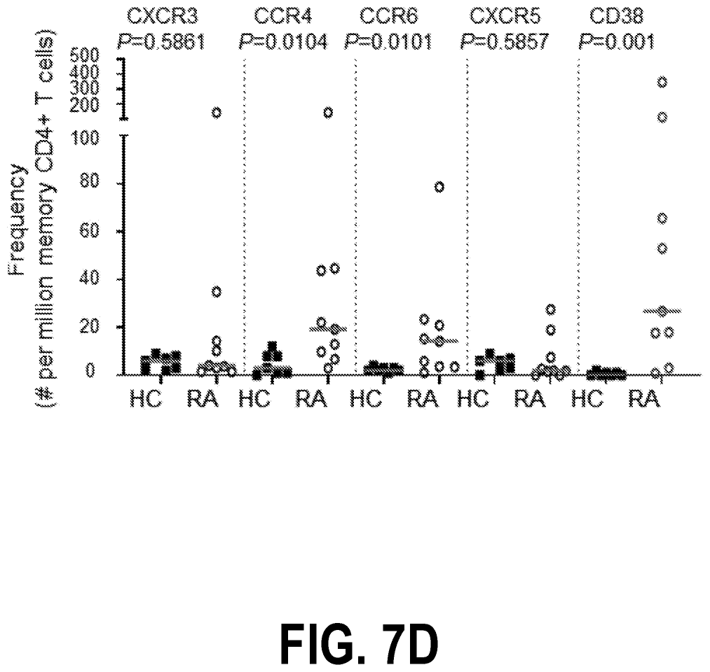

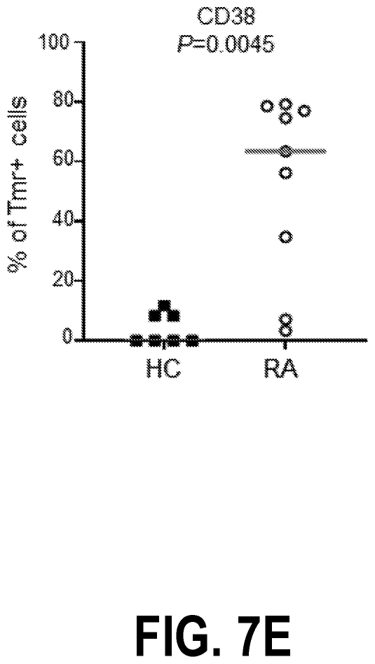

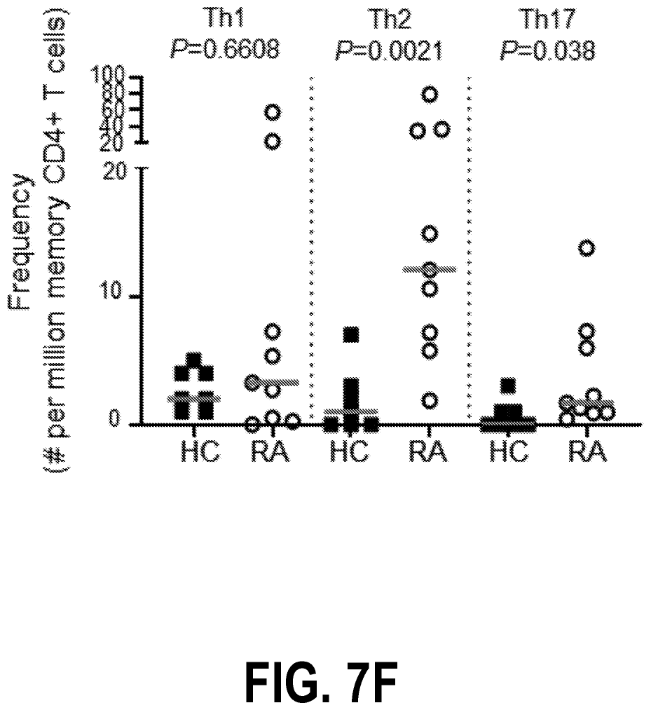

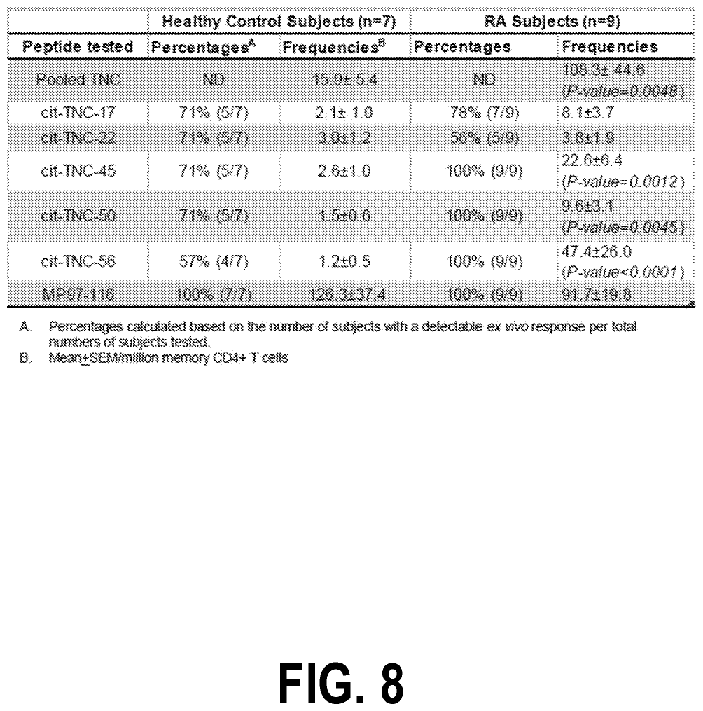

The presence of cit-TNC-specific T cells was evaluated in individuals with RA (n=9) and healthy control (HC) subjects (n=7) (FIG. 6, T cell cohort). The frequency and surface phenotype of the cit-TNC-specific T cells were directly assessed using a multiplex HLA class II tetramer staining approach that allowed ex vivo enrichment and detection of multiple tetramer specificities in a single peripheral blood sample and co-staining with cell surface marker antibodies (Rims et al. 2019 Arthritis Rheumatol. 71:518). The flow cytometry panel included the five immunogenic cit-TNC specificities: cit-TNC17, cit-TNC22, cit-TNC45, cit-TNC50, and cit-TNC56 (SEQ ID NOS: 53, 55, 57, 59, and 61, respectively), plus an influenza peptide (MP 97-116) as a positive control. Combining all cit-TNC specificities, there was a significant increase in the frequency of memory cit-TNC-specific T cells in patients with RA compared to HC subjects (FIG. 7A). In contrast, the frequency of memory influenza-specific T cells did not differ between RA and HC subjects. Notably, cit-TNC45, cit-TNC50 and cit-TNC56, contributed the most to the increased frequency of cit-TNC-specific T cells, with a trend toward higher cit-TNC17-specific T cells that did not reach statistical significance (FIGS. 7B-7C, FIG. 8). The frequency of memory influenza-specific T cells, however, did not differ between RA and HC subjects. The frequencies of cit-TNC45, cit-TNC50 and cit-TNC56 specific T cells were all significantly increased, while the other specificities were only detected in a few of the RA patient samples (FIGS. 7B-C and FIG. 8). As can be seen in FIG. 7C, each patient sample contained several cit-TNC T cell specificities, but with variable frequencies.

### Example 3

Cit-Tenascin-C-Specific T-Cells were Recently Activated and Exhibited Predominantly Th2 and Th17 Phenotypes.

Cell surface phenotype of cit-TNC-specific T cells in peripheral blood was utilized to draw inferences about T cell lineage, observing key differences between rheumatoid arthritis (RA) subjects and healthy control (HC) subjects. The cell surface markers included CD45RA as a marker of naïve T cells, CD38 as a marker of recent activation, and CXCR3, CCR4, CXCR5 and CCR6 to define Th1, Th2, Tfh, and Th17 T helper subsets, respectively (Acosta-Rodriguez et al. 2007 Nat Immunol. 8:639; Becattini et al. 2015 Science. 347:400; Quarona et al. 2013 Cytometry Part B, Clinical cytometry. 84:207). The frequency and percentage of CD38+ cit-TNC-tetramer (Tmr)+CD4 memory T cells were increased in RA compared to HC subjects (FIGS. 7D-7E). On memory cells, this marker has been shown to indicate in vivo activation due to antigen exposure (Malavasi et al. 1992 International journal of clinical & laboratory research. 22:73; Wambre et al. 2012 The Journal of allergy and clinical immunology. 129:544, 5). Therefore, the presence of CD38 suggested that a substantial proportion of cit-TNC specific T cells in RA subjects were recently activated. In addition, there was a significant increase in the frequency of both CCR4+ and CCR6+ cit-TNC-specific T cells in RA subjects compared to HC subjects (FIG. 7D). Further gating of these Tmr+ cells indicated that this represented a significant increase in the Th2 (CCR4+CCR6−CXCR3−) and Th17 (CCR4+CCR6+CXCR3−) population within the RA cohort (FIG. 7F). Therefore, these results suggest that cit-TNC-specific T cells were more frequent in subjects with RA than in controls, and exhibited an activated Th2/Th17 phenotype in peripheral blood.

### Example 4

### Synovial Fluid Mononuclear Cells Secreted Cytokines in Response to Cit-TNC Peptides.

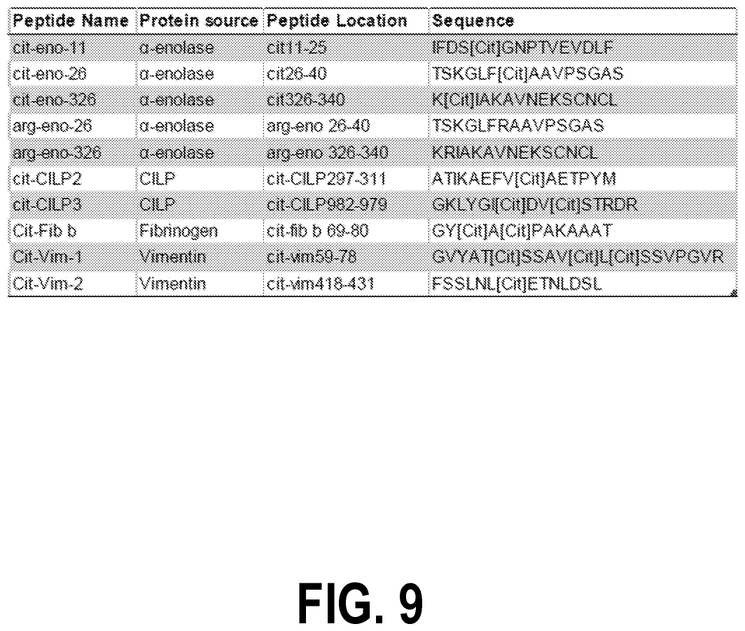

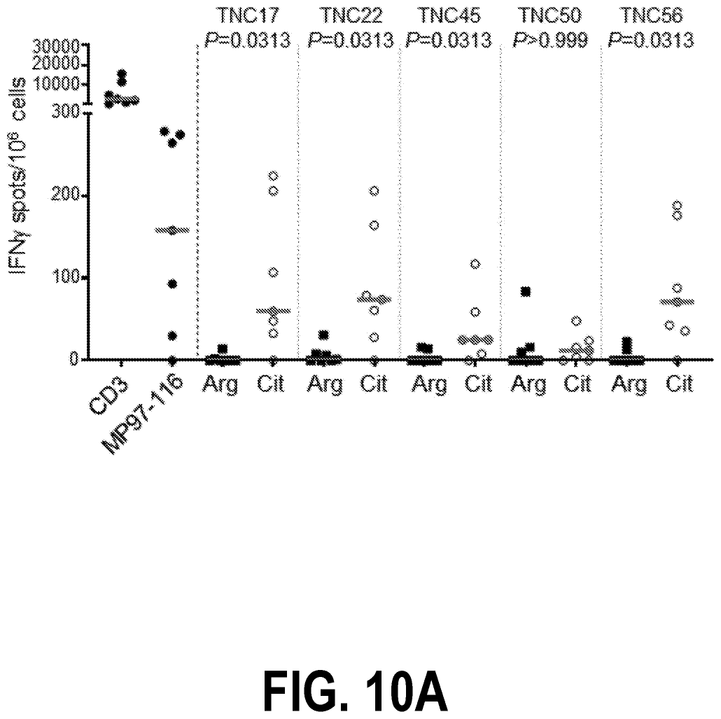

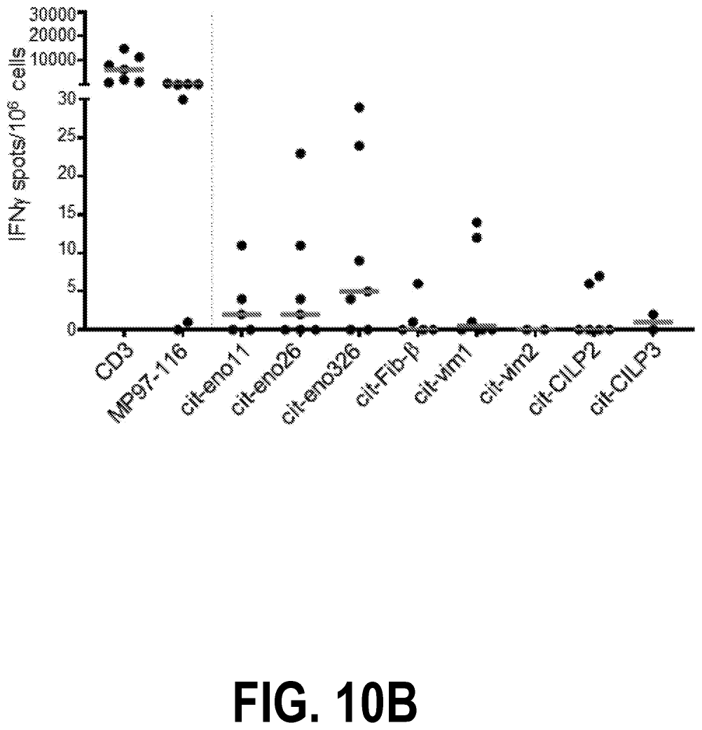

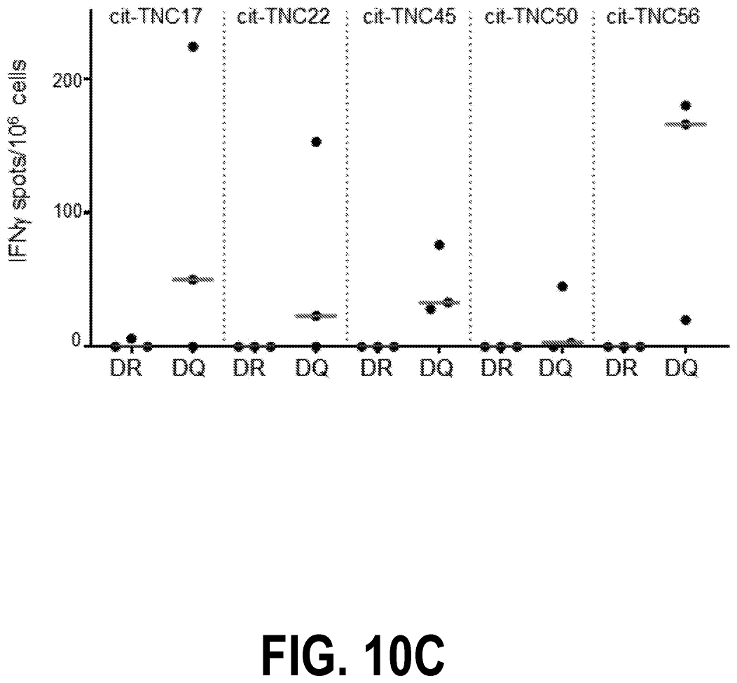

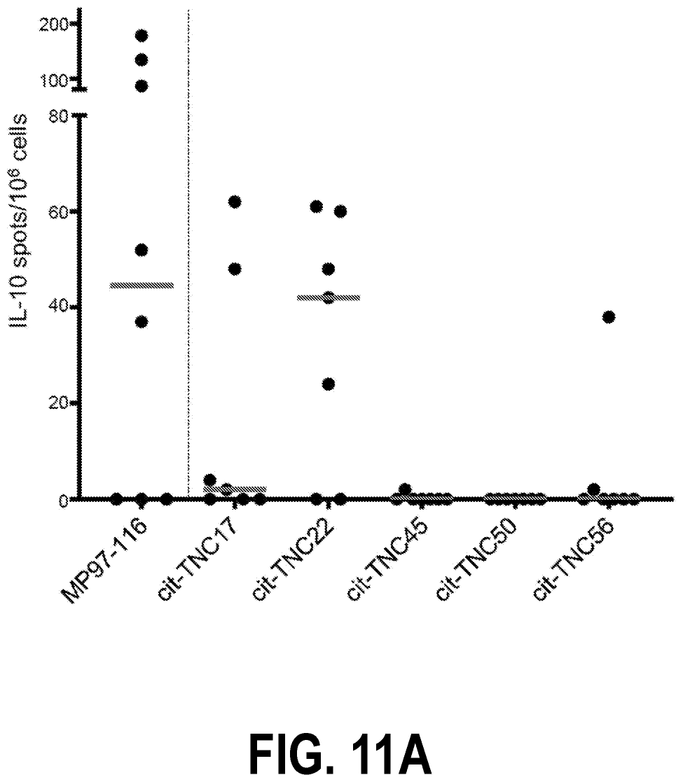

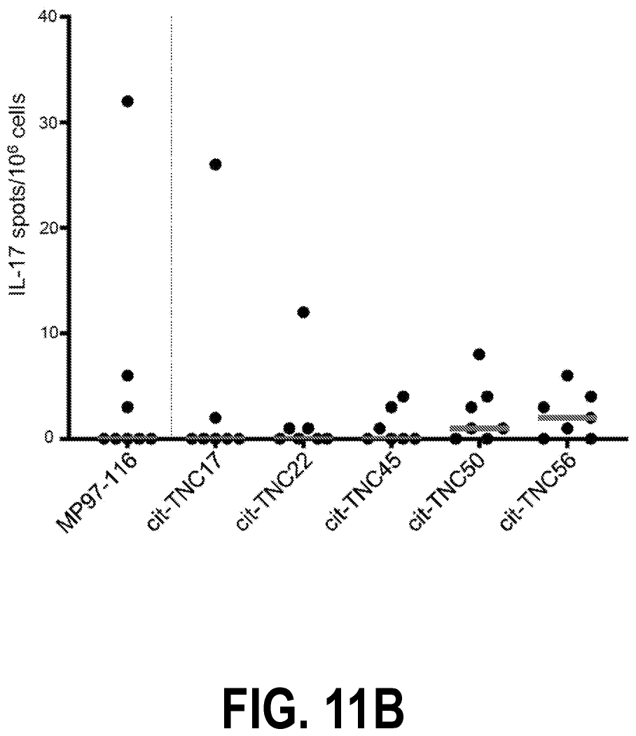

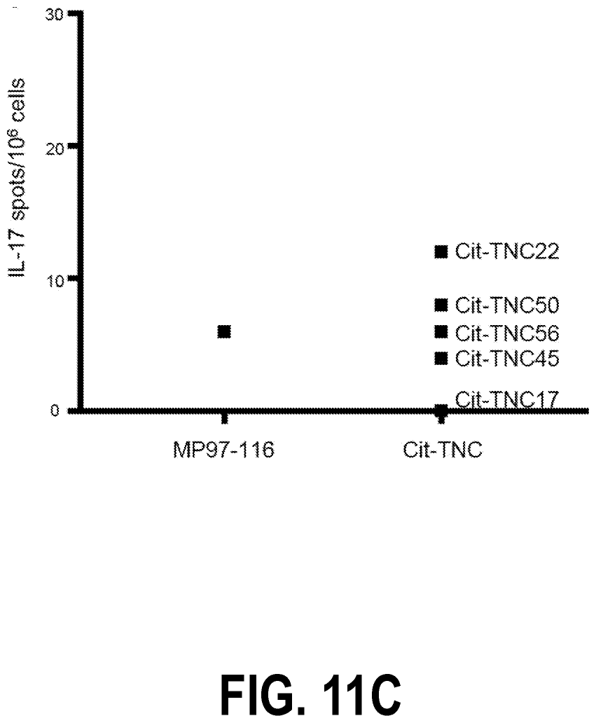

Having established that cit-TNC specific T cells were present in peripheral blood, their presence at disease relevant sites of inflammation were subsequently evaluated. Synovial fluid mononuclear cells (SFMC) from HLA-DRB1*04:01 RA patients (n=7) were stimulated with the five immunogenic cit-TNC peptides or their corresponding arg-TNC peptides, and cytokine secretion (IFN-γ/IL-17/IL-10) was then assessed using a FluoroSpot assay. Citrullinated peptides derived from α-enolase, fibrinogen, vimentin and CILP were included as reference epitopes, since these proteins were previously reported as RA autoantigens (James et al. 2014 Arthritis Rheumatol. 66:1712; Pieper et al. 2018 J Autoimmun. 92:47; Gerstner et al. 2016 Frontiers in immunology. 7:494; Lundberg et al. 2008 Arthritis Rheum. 58:3009; van Steendam et al. 2010 Arthritis Res Ther.12:R132) (FIG. 9). Cit-TNC17, cit-TNC22, cit-TNC45 and cit-TNC56 (but not cit-TNC50) elicited IFN-γ secretion by SFMC (FIG. 10A). Even though arg-TNC peptides could promote peripheral blood T cell expansion, cytokine responses to the unmodified peptides in the synovial fluid samples were rarely observed, while the cit-TNC peptides displayed a robust IFN-γ output (FIG. 10A). The observed responses toward cit-TNC were almost an order of magnitude greater than those seen for cit-enolase, cit-fibrinogen, cit-vimentin and cit-CILP (FIG. 10B)—in some cases approaching the level of response seen for the influenza peptide. The observed SFMC response to cit-TNC peptides was HLA-DR restricted, as HLA-DR blocking, but not HLA-DQ blocking, completely abrogated the induction of IFN-γ secretion (FIG. 10C). The cit-TNC peptides also elicited modest IL-10 and IL-17 secretion by SFMC (including some IFN-γ and IL-10 double positives), although this appeared to be epitope and subject specific (FIGS. 11A-11C).

### Example 5

Cit-Tnc17 and Cit-Tnc56 were Recognized by Antibodies in Ra Subjects

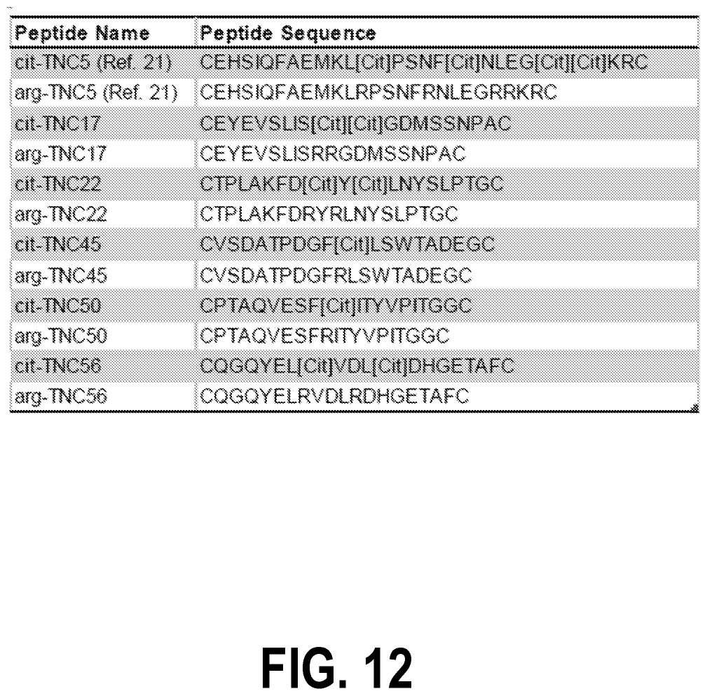

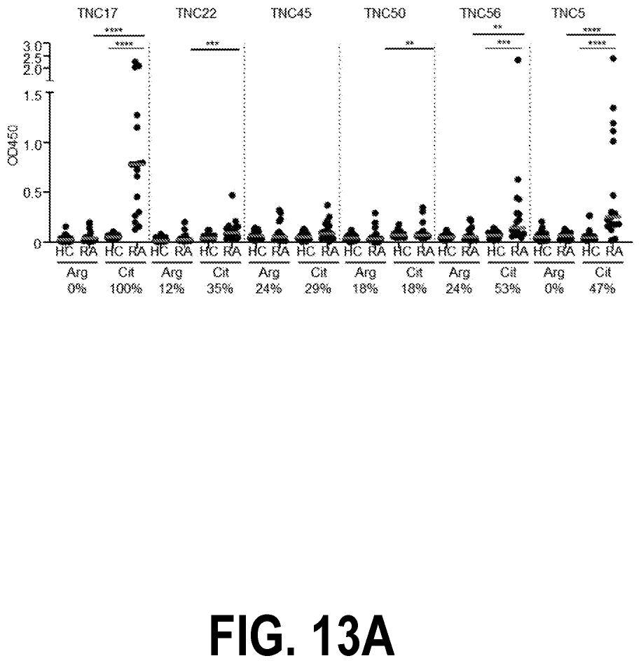

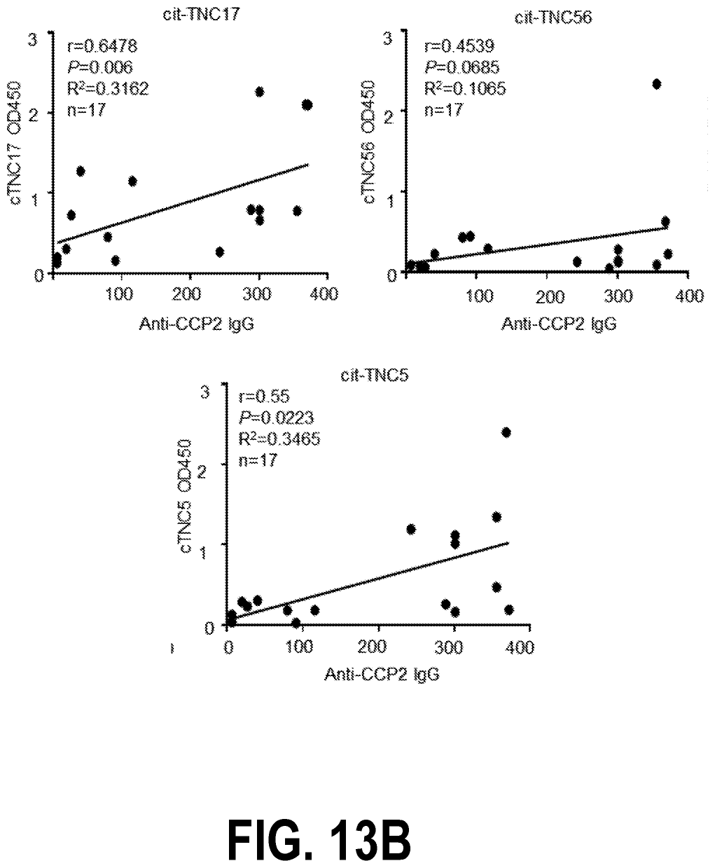

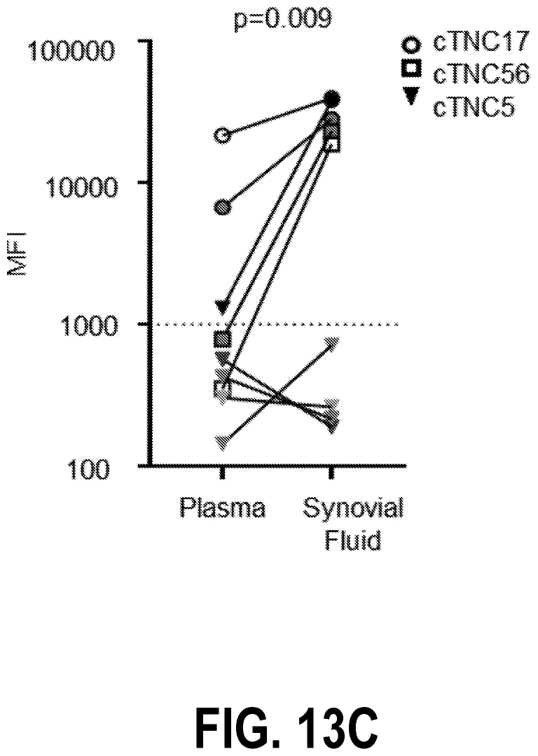

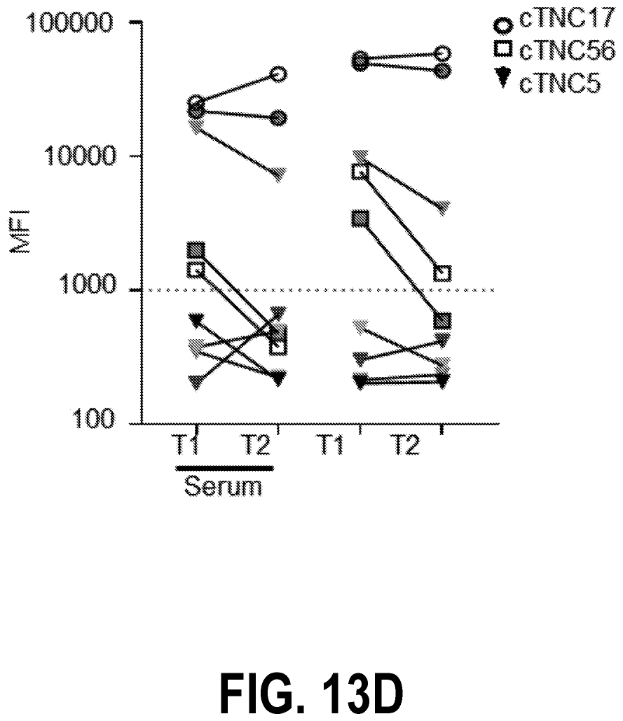

Given the importance of anti-citrullinated protein antibodies (ACPA) as markers for the diagnosis of RA and prior observations that a citrullinated peptide from the C-terminal FBG domain of TNC was recognized by serum ACPA (Riedl et al. 2001 Int J Colorectal Dis. 16:285), the peptides found to promote the T cell response to TNC were evaluated to determine if they also function as antibody epitopes. To assess this property, antibody responses to cit-TNC and arg-TNC peptides (plus the previously published cit-TNC5 peptide as a positive control) were measured in serum samples from HLA-DRB1*04:01 and CCP positive RA (n=17) and HLA-DRB1*04:01 matched HC (n=24) subjects (FIG. 6, Autoantibody Cohort 1 and FIG. 12). This autoantibody cohort had only partial overlap with the T cell cohort. Antibodies to the previously reported cit-TNC5 were present in 47% of the RA sera, consistent with the levels previously seen in two independent cohorts (Schwenzer et al. 2016 Ann Rheum Dis. 75:1876). In contrast, antibodies to cit-TNC17 were present in 100% of the RA sera while antibodies to cit-TNC56, cit-22 and cit-TNC45 were present in 53%, 35%, and 29% of the RA sera, respectively. Antibody titers were highest for TNC17, TNC5, and TNC56 [SEQ ID NOS: 17, 51, 56] respectively, while more modest reactivity was observed for TNC22 and TNC45 [SEQ ID NOS:20, 18] (FIG. 13A). There was little or no antibody reactivity directed against the corresponding arg-TNC peptides (FIG. 13A). Furthermore, antibody reactivity was virtually absent among the HC sera. Together, these observations suggested that TNC was selectively recognized as a citrullinated antigen by antibodies present in the sera from RA patients that were essentially absent from HLA matched controls.

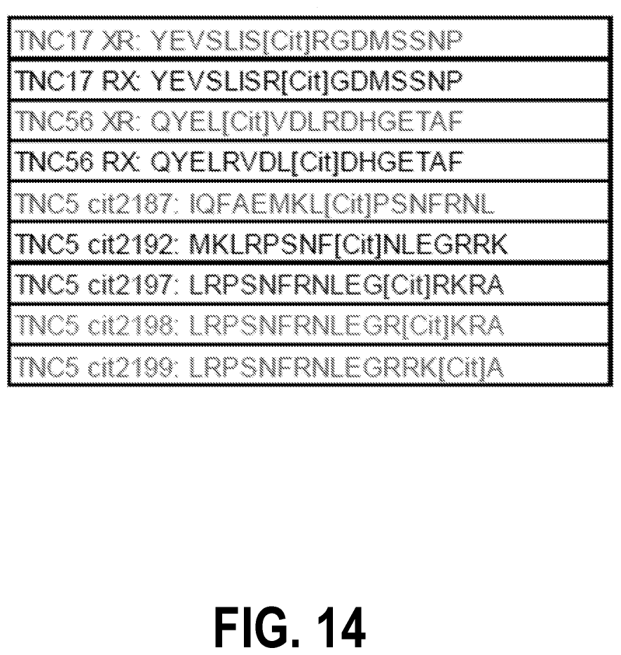

To further probe their potential significance, observed levels of cit-TNC reactive antibodies were correlated with anti-CCP2 antibody levels (FIG. 13B), observing a positive correlation in this cohort between anti-CCP2 and antibody levels for both cit-TNC17 (r=0.6478, p=0.006) and cit-TNC5 (r=0.55, p=0.0223), but that trend did not reach significance for cit-TNC56 (r=0.4539, p=0.0685). Since ACPA are thought to be directly involved in joint pathology, polyclonal CCP-pools purified from both plasma and synovial fluid samples were tested for reactivity to an array of TNC peptides covering every arginine/citrulline residue within the protein (Ossipova et al. 2014 Arthritis Res Ther. 16:R167). Among the peptides tested, reactivity to cit-TNC17, cit-TNC56 and cit-TNC5 in plasma could be verified and a strong positive signal was observed in synovial fluid derived antibody pools (FIG. 13C and FIG. 14). The observation of elevated levels of TNC reactivity in synovial fluid versus plasma was further replicated in a set of individually paired serum and synovial fluid samples taken 10 years apart, suggesting that cit-TNC antibodies persisted over time (FIG. 13D and FIG. 14).

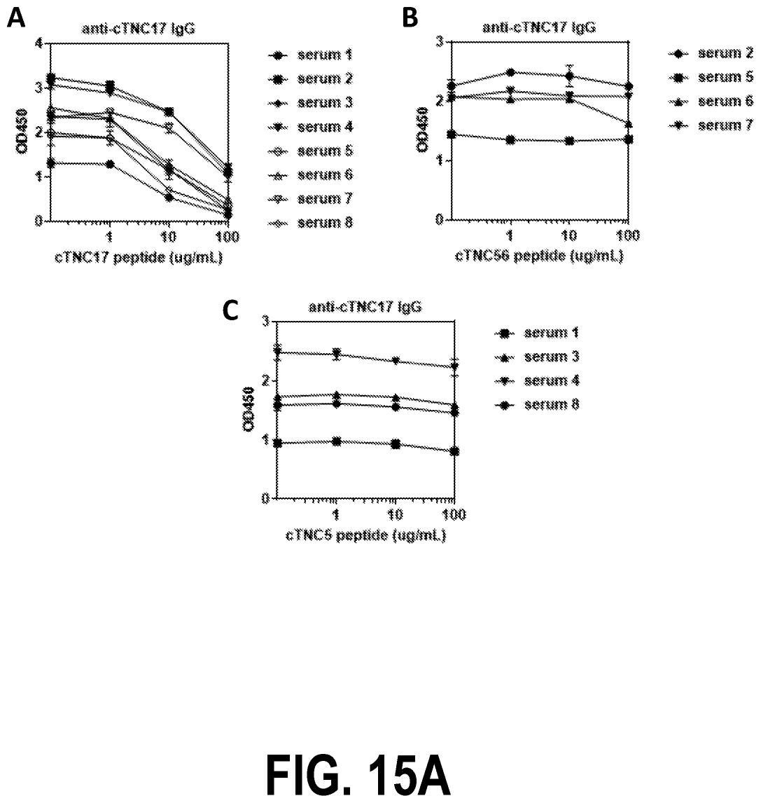

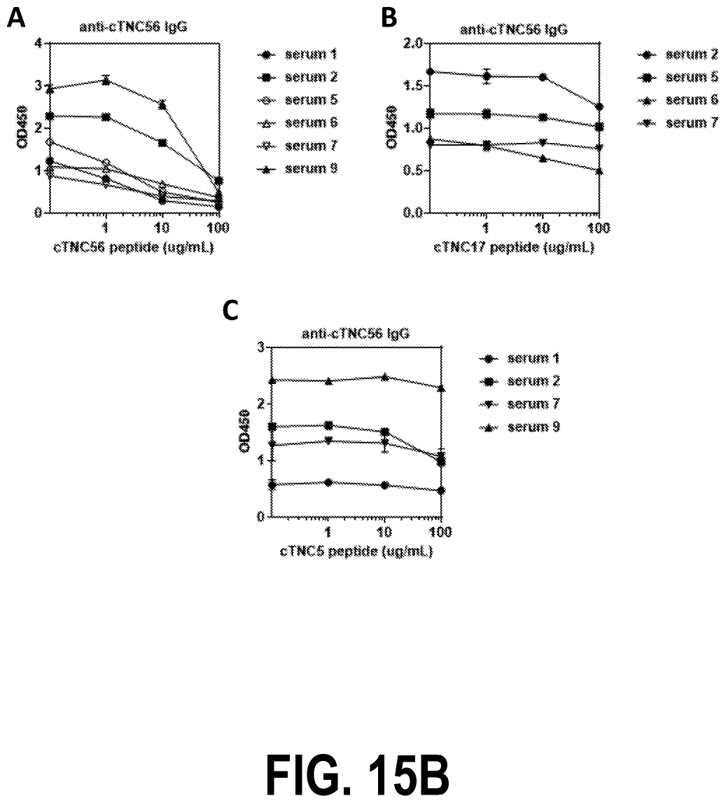

Since it has been recently shown that ACPA can cross-react against multiple peptides (especially those that contain a common citrulline-glycine motif (Sahlström et al. 2020 Arthritis Rheumatol. 72:1643), the specificity and potential cross-reactivity between cit-TNC17 and cit-TNC56 reactive antibodies was examined by performing inhibition experiments using sera from subjects that were double-reactive for cit-TNC17 and cit-TNC56 antibodies. Absorption by the homologous peptide was efficient for both cit-TNC17 and cit-TNC56, (FIG. 15A-15B, panel A), whilst cross-reactivity was limited; only partial inhibition was observed in serum 2 (maximum inhibition 24.8%: 56 vs 17) and serum 6 (maximum inhibition 22%: 17 vs 56 and 43% for 56 vs 17). (FIG. 15A-15B, panel B). There was also no cross-reactivity between cit-TNC17 and cit-TNC5, and only limited cross-reactivity between cit-TNC56 and cit-TNC5 (maximum inhibition of serum 2:38.7%) (FIG. 15A-15B, panel C). Cumulatively, this minimal cross-reactivity suggested that distinct antibodies recognized these different cit-TNC peptides.

### Example 6

Cit-Tnc17 and Cit-Tnc56 Antibodies were Associated with Clinical Features of Ra

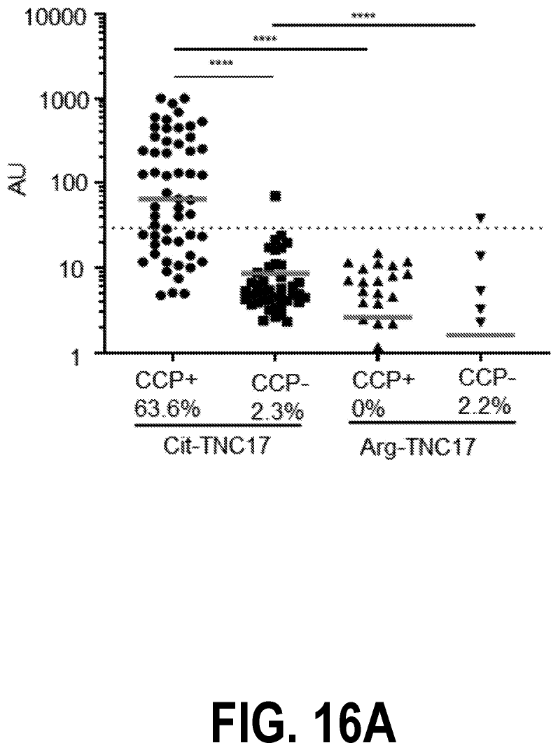

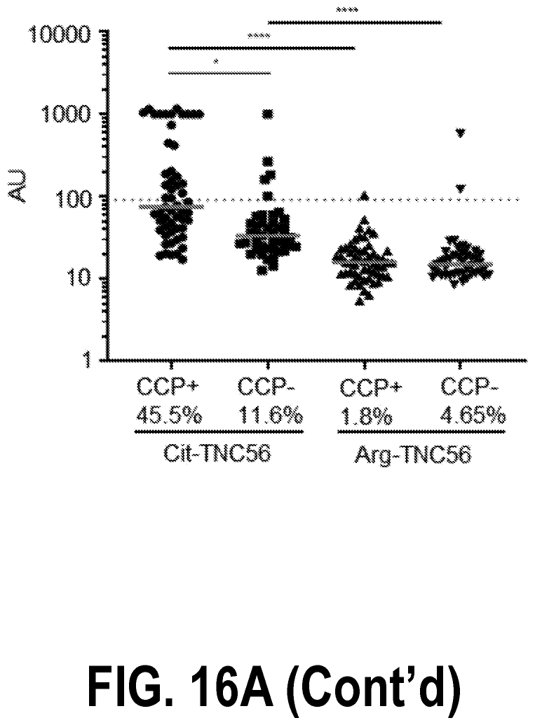

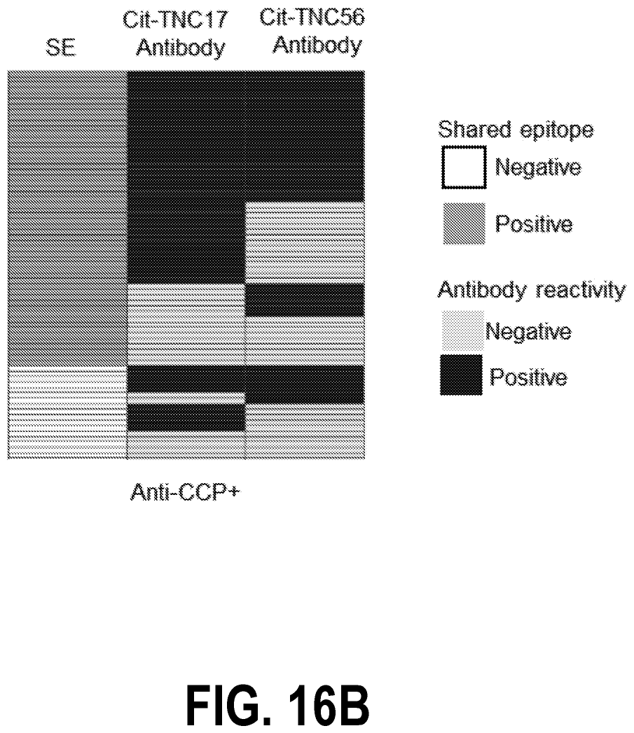

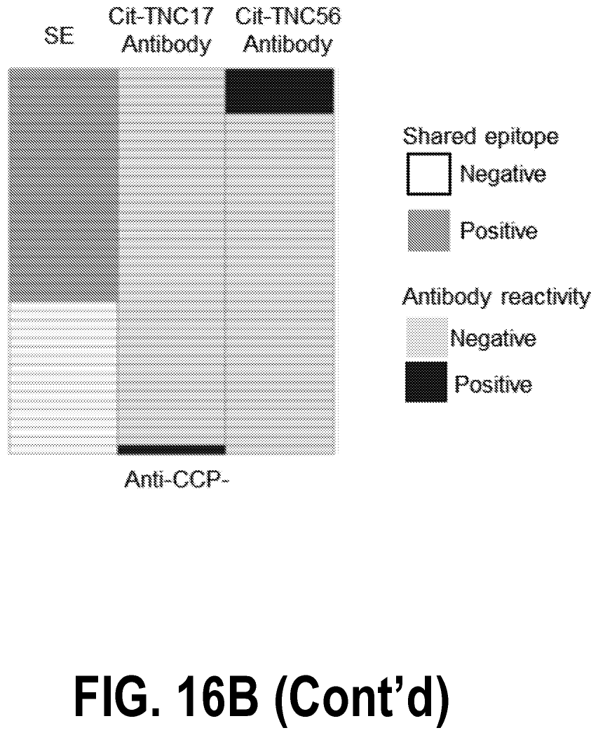

Given the prevalence of antibody reactivity toward cit-TNC17 and cit-TNC56 in subjects with RA, levels of these antibodies were investigated to determine if they correlated with specific clinical features. To facilitate this analysis, cit-TNC seropositivity was examined in an independent cohort of RA subjects. This cohort included CCP+RA (n=55) and CCP−RA (n=43) subjects and who were either positive or negative for the HLA DRB1*04:01 haplotype (FIG. 6, Autoantibody Cohort 2). In this cohort, cit-TNC17 antibodies were present in 64% of CCP+ subjects but only 2.3% of CCP− subjects, and cit-TNC56 antibodies were present in 46% of the CCP+ cohort and 12% of the CCP− subjects (FIG. 16A). The antibody reactivity for arg-TNC17 and arg-TNC56 peptides was below the threshold for positivity in almost all subjects (FIG. 16A). Therefore, data from this patient cohort supported preferred antibody reactivity toward cit-TNC and an association with cyclic citrullinated peptide (CCP) positivity.

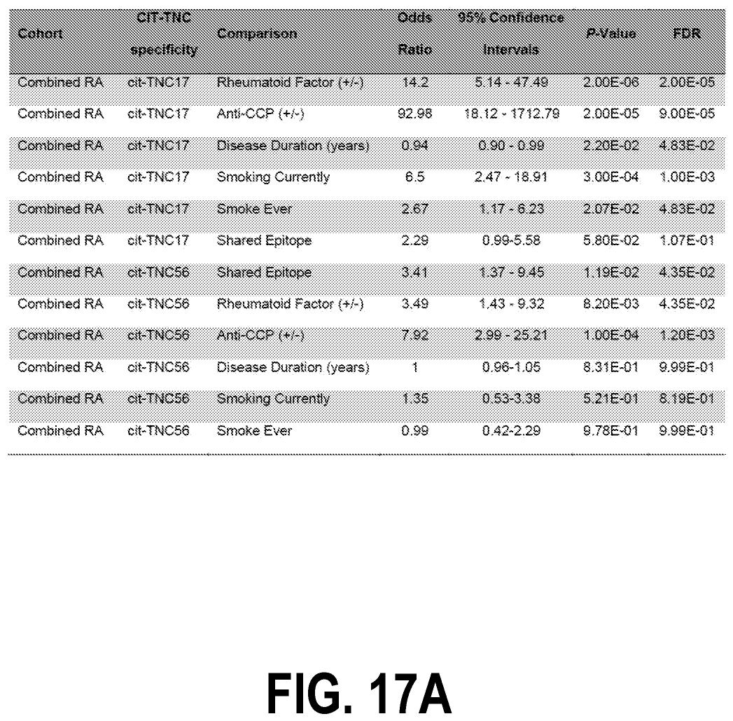

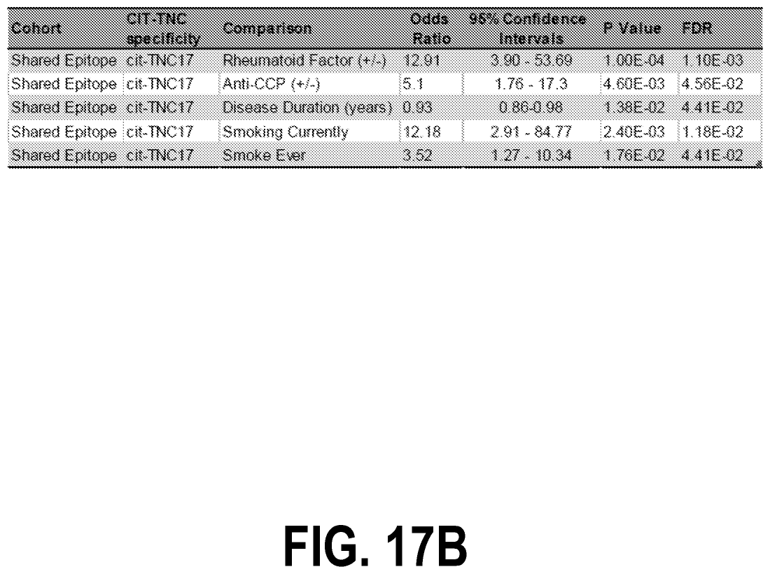

The clinical and serologic data from Autoantibody Cohorts 1 and 2 were combined to maximize the ability to assess relationships between cit-TNC17 and cit-TNC56 reactive antibodies with specific features of RA. In the combined cohort 79% of the CCP+ subjects exhibited positive reactivity toward at least one of the cit-TNC peptide. Of these seropositive subjects, 28% were single positive for anti-cit-TNC17 antibodies, 11% were single positive for anti-cit-TNC56 antibodies, 40% were dual positive for both antibodies (FIG. 16B). Furthermore, there was a positive association between cit-TNC seropositivity and the HLA shared epitope (SE) (FIG. 16B). This result was further confirmed using a logistic regression model in which age and sex were used as co-variates to calculate odds ratios (OR) (FIG. 17A).

In addition, cit-TNC17 seropositivity was strongly associated with both rheumatoid factor (OR=14.2, FDR=0.00002) and CCP seropositivity (OR=92.98, FDR=0.00009) (FIG. 17A). To a lesser extent, cit-TNC56 seropositivity was also associated with rheumatoid factor (OR=3.49, FDR=0.04345) and CCP seropositivity (OR=7.92, FDR=0.0012) (FIG. 17A). cit-TNC17 seropositivity was also associated with smoking (current smoking OR=6.5, FDR 0.001) (FIG. 17A), but not associated with disease activity and disease duration. Applying the model exclusively to shared epitope positive RA subjects, the associations between cit-TNC17 seropositivity and rheumatoid factor, CCP seropositivity and smoking remained strong and significant (FIG. 17B). The association of antibodies against cit-TNC17 and TNC56 with the shared epitope, smoking and rheumatoid factor mirrored the characteristics described in ACPA+ subjects who go on to develop RA (Rantapaa-Dahlqvist et al. 2003 Arthritis Rheum. 48:2741; van Beers et al. 2012 Arthritis Res Ther. 14:R35; Silman et al. 1996 Arthritis Rheum. 39:732; Wagner et al. 2015 Ann Rheum Dis. 74:579; Stolt et al. 2003 Ann Rheum Dis. 62:835; Hensvold 2015 et al. Ann Rheum Dis. 74:375; Mahdi et al. 2009 Nat Genet. 41:1319; de Vries-Bouwstra et al. 2008 Arthritis Rheum.58:1293; Huizinga et al. 2005 Arthritis Rheum.; 52(11):3433-8). Therefore, the results suggested that cit-TNC responsiveness was most pronounced in subjects with high risk HLA genotypes and who have a history of smoking.

### Example 7

### Materials and Methods

### Human Subjects

All RA subjects met the 2010 American College of Rheumatology (ACR)/European League Against Rheumatism (EULAR) 2010 Rheumatoid Arthritis Classification Criteria and had at least one HLA-DRB1*04:01 allele, and ACPA positivity was determined based on clinical testing for CCP. All HC subjects had no history of autoimmune disease themselves or among their first degree relatives, had at least one HLA-DRB1*04:01 allele, and were ACPA negative. The characteristics of each of the RA and HC cohorts are summarized in FIG. 6. PBMC and serum samples from both RA and HC subjects were from the Benaroya Research Institute Immune-Mediated Disease Registry and Repository (Seattle, WA). Synovial fluid samples were obtained from patients with RA undergoing arthrocentesis as part of clinical treatment at the Karolinska University Hospital. For the study testing polyclonal CCP-pools purified from both plasma/serum and synovial fluid samples for reactivity to an array of TNC peptides, the plasma and serum samples were obtained from RA patients attending the Karolinska University Hospital as previously described (Steen et al., 2019 Arthritis Rheumatol. 71:196, James et al. 2010 Arthritis Rheum. 10:2909).

### Isolation and Cryopreservation of PBMC and SFMC

PBMC and SFMC were isolated from heparinized blood and synovial fluid respectively by centrifugation over Ficoll-Hypaque gradients, and frozen in liquid nitrogen in 10% DMSO and 90% heat-inactivated FBS. Cryopreserved PBMC were thawed in a 37° C. water bath and prepared by drop-wise addition of RPMI-1640 media supplemented with 10% FBS and 0.001% benzonase nuclease (Sigma-Aldrich), with a final suspension in complete medium (RPMI-1640 supplemented with 2 mM L-glutamine, 100 U/ml penicillin, 100 μg/ml streptomycin, 10 mM HEPES) supplemented with 10% commercial human pooled serum (HPS). SFMC were thawed in a 37° C. water bath and rapidly resuspended in complete medium with ≥5 U/ml benzonase nuclease (Sigma-Aldrich) and 10% FBS. Before in vitro stimulation, the cells were resuspended in complete medium with 10% HPS (Sigma-Aldrich).

### Epitope Prediction and Peptide Synthesis

Putative cit-TNC epitopes were predicted using the scanning algorithm as previously described (James et al. 2014 Arthritis Rheumatol. 66:1712, James et al. 2010 Arthritis Rheum. 62:2909). Briefly, motif scores were calculated by multiplying coefficients corresponding to each anchor residue for all possible core 9-mers within the protein that included an internal or flanking arginine or citrulline residue. A total of 64 peptides with motif scores of 0.1 or higher were synthesized and purified by the manufacturer (Sigma-Aldrich). All peptides were dissolved in DMSO to a stock concentration of 20 mg/ml. For tetramer production and further studies, selected citrullinated peptides and their corresponding native peptides were resynthesized by Sigma-Aldrich, GenScript or Pepceuticals.

### Peptide Binding to HLA-DRB1*04:01

To assess peptide binding to HLA-DRB1*04:01, increasing concentrations of each non-biotinylated test peptide were incubated in competition with 0.01 μM biotinylated influenza HA306-318 (PKYVKQNTLKLAT) (SEQ ID NO: 16) in wells coated with anti-HLA-DR antibody (Clone L243, supplied by the BRI Tetramer Core) as previously described (Ettinger et al. 2006 J Immunol. 176:1988). Europium-conjugated streptavidin (PerkinElmer) was used to label residual biotinylated peptide bound to the HLA-DR protein and was quantified using a Victor2 multi-label time resolved-fluorometer (PerkinElmer). Binding curves were fitted by non-linear regression with a sigmoidal dose response curve mode using GraphPad Prism 7.0 and EC50 values were calculated as the peptide concentration needed to displace 50% of the reference peptide. Peptides selected for further study based on positive binding results were re-synthesized at a higher purity by another manufacturer (GenScript or Pepceuticals).

### HLA Class-II Tetramer Production

Recombinant HLA-DRB1*04:01 protein was produced by the BRI Tetramer Core (Benaroya Research Institute, Seattle, WA) as previously described (Novak et al. 1999 J Clin Inv.104:R63). Soluble HLA-DRB1*04:01 monomer was purified from insect cell culture supernatants and biotinylated at a sequence-specific site using biotin ligase (Avidity) prior to dialysis into phosphate storage buffer. The biotinylated monomer was loaded with 0.2 mg/ml of peptide by incubating at 37° C. for 72 hours in the presence of 2.5 mg/ml n-octyl-β-D-glucopyranoside and 1 mM Pefabloc SC (Sigma-Aldrich). Peptide loaded monomers were conjugated into tetramers using fluorescently labeled streptavidin (Invitrogen) for 6-18 hours at room temperature at a molar ratio of 8:1.

### In Vitro Expansion Cit-TNC-Specific T Cells and HLA Class-II Tetramer Staining

Cryopreserved PBMCs from RA and healthy subjects were cultured at 5×10⁶ cells/well in a 48 well plate in RPMI-1640+10% HPS with 10 μg/ml of peptide. IL-2 (Novartis) was added at 325 IU/ml on day 6. On day 14, cells were stained for expression of tetramer-PE, CD25 APC (BD) and CD4 FITC (BioLegend), and then characterized by immunocytofluorimetry on a FACSCanto™ (BD). The data were analyzed by FlowJo software version 10.

### SFMC Secretion of Cytokines in Response to Cit-TNC Peptides

Cryopreserved SFMC from seven patients were stimulated with cit-TNC17, cit-TNC 22, cit-TNC 45, cit-TNC 50, cit-TNC56 peptides and their arginine counterparts. Peptide stimulation with citrullinated α-enolase, vimentin, CILP and fibrinogen peptides was also performed as a comparison for four of these seven patients (n=2 at the same time-point, n=2 at a different time-point and sample), and additionally for three patients. Non-TNC peptides used in the assay were synthesized by GenScript and are described in FIG. 9. One additional patient was only included in a TNC HLA-blocking experiment. A three-color fluorospot assay was performed using the Mabtech IFN-γ/IL-10/IL-17 kit for all tenascin-C experiments and the IFNγ/IL-22/IL-17 kit for the additional experiments with other citrullinated peptides. Briefly, SFMC were thawed and 250-850 thousand cells were plated per well, in complete medium with 10% human serum (Sigma-Aldrich), in a pre-coated fluorospot plate and stimulated with 20 μg/ml of the separate peptides for 48 hours at 37° C., 5% CO₂. 20 μg/ml influenza peptide (MP97-116) and anti-CD3 were used as positive control. 10 μg/ml HLA-DR (BioLegend) and HLA-DQ (Beckman Coulter) blocking antibodies were used in addition to the cit-TNC peptides for three patients. After incubation, the plate was washed in PBS and stained with antibodies according to the manufacturer's protocol. The plates were read using an ELISpot/Fluorospot reader (iSpot Spectrum, AID, Strassberg, Germany), software version 7.0 (build 151117) with automated spot count. The number of spots was normalized to spots per million cells, and the number of spots seen in the unstimulated wells was subtracted from the spot count in the stimulated wells prior to further analyses. The Wilcoxon matched pairs signed rank test was used for the fluorospot assays and p<0.05 was considered significant.

### Ex Vivo Detection of Flu and Cit-TNC-Reactive T Cells by HLA Class-II Tetramers

Ex vivo tetramer staining and enrichment was accomplished using previously published protocols (Rims et al. 2019 Arthritis Rheumatol. 71:518; Uchtenhagen et al. 2016 Nat Comm 7:12614). A total of 40-60 million PBMCs were thawed and rested overnight in a tissue culture incubator at 37° C., 5% CO₂. PBMCs were equally divided into two FACS tubes for MP97-116 and citrulline TNC Tetramer tests, each in 200 μl of T cell culture medium following Dasatinib treatment for 10 minutes at 37° C. PBMCs were then stained with 6 μl of each of TNC-PE, TNC-PE-CY5, TNC-PE-CF or MP97-116-BV421-labeled tetramers at room temperature for 90 minutes. Cells were washed and incubated with 40 μl anti-PE and 10 μl anti-Myc magnetic beads (Miltenyi Biotec) at 4° C. for 20 minutes, washed again, and a 1/100th fraction was saved for antibody staining (“Pre”). The other fraction was passed through a MS magnetic column (Miltenyi Biotec). Bound, PE, PE-CY5, PE-CF or BV421-labeled cells were flushed and collected. Both enriched (“Post”) and non-enriched (“Pre”) fractions were labeled with Sytox, CD4 V500, CD45RA AF700, CD38 BUV395 (BD Bioscience), CXCR3 AF647, CCR4 BV605, CXCR5 PE-Cy7, CCR6 BV650, CCR7 APC-Cy7, CD14 FITC and CD19 FITC (BioLegend). Samples were run on a BD LSRII flow cytometer, and data was analyzed using FlowJo software version 10. The frequency (F) of epitope-specific T cells per million memory CD4+ T cells was calculated as follows: F=(1,000,000×tetramer-positive events from enriched tube)/(100×number of memory CD4+ T cells from the “Pre” fraction). Single T cells were gated CD4+/CD14-CD19-sytox- and a triple-negative population was gated for each of the three possible tetramer fluorophores. These served as the parent populations from which one of the four tetramer positive T-cell populations were then gated on and analyzed for surface receptor expression. T-cell lineage was assigned on CD45RA− memory cells as follows: Th1 (CXCR3+, CCR4− and CCR6−); Th2 (CXCR3−, CCR4+ and CCR6−); Th17 (CXCR3−, CCR4+ and CCR6+); Th1*(CXCR3+, CCR4− and CCR6+) (24, 25).

### ELISAs for Detection of Antibodies

ELISAs were used to detect antibodies against citrullinated peptides in sera from patients in Autoantibody Cohorts 1 and 2 (FIG. 6). Peptides used in the assay were synthesized by either GenScript or Pepceutical and are described in FIG. 12. The ELISAs were conducted as previously described (Riedl et al. 2001 Int J Colorectal Dis.16:285; Quarona et al. 2013 Cytometry Part B, Clinical cytometry 84:207). In brief, 96-well Nunc Maxisorp plates were coated with 10 μg/ml peptide in PBS, blocked with 2% BSA and incubated with sera diluted 1:100. Bound antibodies were detected with an HRP-conjugated anti-human IgG Fc monoclonal antibody (6043HRP, Stratech). A standard curve of positive sera was used to calculate relative antibody titers in arbitrary units (AU) for each sample. The CCP2 ELISA for Autoantibody Cohort 1 was performed as by the manufacturer's instruction (FCCP600, Axis-Shield). Cross-reactivity was analyzed in human sera that were reactive to both cTNC56 or cTNC17 and cTNC5. Sera were diluted 1:100, incubated with 1, 10 and 100 μg/ml of peptides for 2 hours, centrifuged at 10,000 g for 10 min and the supernatant added to peptide-coated plates for analysis by ELISA as described above. Positivity was defined by the cut-off of the 98% percentile of healthy control samples from Autoantibody Cohort 1.

### Extracellular Matrix Peptide Microarray.

The custom-made microarray was performed as previously described (Steen et al. 2019 Arthritis Rheumatol. 71:196). In brief, 16-aa long peptides that covered all arginine residues of 1,610 extracellular matrix proteins and RA related proteins were synthesized in situ (Roche NimbleGen). For tenascin C, there were a total of 217 peptides including both native and citrullinated variants of each peptide synthesized. Samples tested for reactivity against TNC derived peptides included synovial and plasma ACPA pools containing CCP-reactive antibodies from 26 and 38 RA patients, respectively (Ossipova et al. 2014 Arthritis Res Ther. 16:R167; Sahlström et al. 2020 Arthritis Rheumatol. 72:1643) Paired synovial fluid and serum samples from one RA patient taken at two time points 10 years apart were also screened. ACPA pools were run at a concentration of 15 μg/ml, whereas synovial fluid and plasma or serum samples were diluted 1/100 and tested for citrulline reactivity using a NimbleGen MS200 Scanner (Roche NimbleGen). A peptide signal intensity variation (spot size) of pixels was used as a basis for calculating the median fluorescence intensities. The cutoff for positive signals was defined as 5 times the fluorescence intensity of the 98th percentile of values for a set of monoclonal antibodies without citrulline reactivity. These were included in the same experiment and were tested at the same time.

### Statistics

All statistical tests were performed using GraphPad Prism version 7.02. Tests that were used (as appropriate) included unpaired T tests, Wilcoxon singed ranked test, Spearman correlations, Mann-Whitney tests, and Kruskal Wallis test with Dunn's multiple comparison test. P values <0.05 were considered significant. To determine the association of tenascin-C seropositivity with clinical variables—including smoking, HLA-DRB1 SE, Rapid3 disease activity scores, disease duration, and PTPN22 C1858T SNP, odds ratios (OR) with 95% Wald confidence intervals (95% CI) were calculated using logistic regression models including age and sex as covariates. p-values were corrected for multiple testing with the Benjamini & Hochberg method.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/109,768, filed on Nov. 4, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES CITED

Acosta-Rodriguez E V, Rivino L, Geginat J, Jarrossay D, Gattorno M, Lanzavecchia A, et al. Surface phenotype and antigenic specificity of human interleukin 17-producing T helper memory cells. Nat Immunol. 2007; 8(6):639-46.
Becattini S, Latorre D, Mele F, Foglierini M, De Gregorio C, Cassotta A, et al. T cell immunity. Functional heterogeneity of human memory CD4(+) T cell clones primed by pathogens or vaccines. Science. 2015; 347(6220):400-6.
de Vries-Bouwstra J K, Goekoop-Ruiterman Y P, Verpoort K N, Schreuder G M, Ewals J A, Terwiel J P, et al. Progression of joint damage in early rheumatoid arthritis: association with HLA-DRB1, rheumatoid factor, and anti-citrullinated protein antibodies in relation to different treatment strategies. Arthritis Rheum. 2008; 58(5):1293-8.
Ettinger R A, Papadopoulos G K, Moustakas A K, Nepom G T, and Kwok W W. Allelic variation in key peptide-binding pockets discriminates between closely related diabetes-protective and diabetes-susceptibleHLA-DQB1*06 alleles. J Immunol. 2006; 176(3):1988-98.
Gerstner C, Dubnovitsky A, Sandin C, Kozhukh G, Uchtenhagen H, James E A, et al. Functional and Structural Characterization of a Novel HLA-DRB1*04:01-Restricted alpha-Enolase T Cell Epitope in Rheumatoid Arthritis. Frontiers in immunology. 2016; 7:494.
Hensvold A H, Magnusson P K, Joshua V, Hansson M, Israelsson L, Ferreira R, et al. Environmental and genetic factors in the development of anticitrullinated protein antibodies (ACPAs) and ACPA-positive rheumatoid arthritis: an epidemiological investigation in twins. Ann Rheum Dis. 2015; 74(2):375-80.
Huizinga T W, Amos C I, van der Helm-van Mil A H, Chen W, van Gaalen F A, Jawaheer D, et al. Refining the complex rheumatoid arthritis phenotype based on specificity of the HLA-DRB1 shared epitope for antibodies to citrullinated proteins. Arthritis Rheum. 2005; 52(11):3433-8.
James E A, Moustakas A K, Bui J, Papadopoulos G K, Bondinas G, Buckner J H, et al. HLA-DR1001 presents “altered-self” peptides derived from joint-associated proteins by accepting citrulline in three of its binding pockets. Arthritis Rheum. 2010; 62(10):2909-18.
James E A, Rieck M, Pieper J, Gebe J A, Yue B B, Tatum M, et al. Citrulline-specific Th1 cells are increased in rheumatoid arthritis and their frequency is influenced by disease duration and therapy. Arthritis Rheumatol. 2014; 66(7):1712-22.
Lundberg K, Kinloch A, Fisher B A, Wegner N, Wait R, Charles P, et al. Antibodies to citrullinated alpha-enolase peptide 1 are specific for rheumatoid arthritis and cross-react with bacterial enolase. Arthritis Rheum. 2008; 58(10):3009-19.
Mahdi H, Fisher B A, Kallberg H, Plant D, Malmström V, Rönnelid J, et al. Specific interaction between genotype, smoking and autoimmunity to citrullinated alpha-enolase in the etiology of rheumatoid arthritis. Nat Genet. 2009; 41(12):1319-24.
Malavasi F, Funaro A, Alessio M, DeMonte L B, Ausiello C M, Dianzani U, et al. CD38: a multi-lineage cell activation molecule with a split personality. International journal of clinical & laboratory research. 1992; 22(2):73-80.
Midwood K S, and Orend G. The role of tenascin-C in tissue injury and tumorigenesis. J Cell Commun Signal. 2009; 3(3-4):287-310.
Novak E J, Liu A W, Nepom G T, and Kwok W W. MHC class II tetramers identify peptide-specific human CD4(+) T cells proliferating in response to influenza A antigen. The Journal of Clinical Investigation. 1999; 104(12): R63-7.
Ossipova E, Cerqueira C F, Reed E, Kharlamova N, Israelsson L, Holmdahl R, et al. Affinity purified anti-citrullinated protein/peptide antibodies target antigens expressed in the rheumatoid joint. Arthritis Res Ther. 2014; 16(4):R167.
Pieper J, Dubnovitsky A, Gerstner C, James E A, Rieck M, Kozhukh G, et al. Memory T cells specific to citrullinated alpha-enolase are enriched in the rheumatic joint. J Autoimmun. 2018; 92:47-56.
Quarona V, Zaccarello G, Chillemi A, Brunetti E, Singh V K, Ferrero E, et al. CD38 and CD157: a long journey from activation markers to multifunctional molecules. Cytometry Part B, Clinical cytometry. 2013; 84(4):207-17.
Rantapaa-Dahlqvist S, de Jong B A, Berglin E, Hallmans G, Wadell G, Stenlund H, et al. Antibodies against cyclic citrullinated peptide and IgA rheumatoid factor predict the development of rheumatoid arthritis. Arthritis Rheum. 2003; 48(10):2741-9.
Riedl S, Tandara A, Reinshagen M, Hinz U, Faissner A, Bodenmuller H, et al. Serum tenascin-C is an indicator of inflammatory bowel disease activity. Int J Colorectal Dis. 2001; 16(5):285-91.
Rims C, Uchtenhagen H, Kaplan M J, Carmona-Rivera C, Carlucci P, Mikecz K, et al. Citrullinated Aggrecan Epitopes as Targets of Autoreactive CD4+ T Cells in Patients With Rheumatoid Arthritis. Arthritis Rheumatol. 2019; 71(4):518-28.
Sahlström P, Hansson M, Steen J, Amara K, Titcombe P J, Forsström B, et al. Different hierarchies of anti-modified protein autoantibody reactivities in rheumatoid arthritis. Arthritis Rheumatol. 2020. 72(10):1643-1657
Schwenzer A, Jiang X, Mikuls T R, Payne J B, Sayles H R, Quirke A M, et al. Identification of an immunodominant peptide from citrullinated tenascin-C as a major target for autoantibodies in rheumatoid arthritis. Ann Rheum Dis. 2016; 75(10):1876-83.
Silman A J, Newman J, and MacGregor AJ. Cigarette smoking increases the risk of rheumatoid arthritis. Results from a nationwide study of disease-discordant twins. Arthritis Rheum. 1996; 39(5):732-5.
Steen J, Forsström B, Sahlström P, Odowd V, Israelsson L, Krishnamurthy A, et al. Recognition of Amino Acid Motifs, Rather Than Specific Proteins, by Human Plasma Cell-Derived Monoclonal Antibodies to Posttranslationally Modified Proteins in Rheumatoid Arthritis. Arthritis Rheumatol. 2019; 71(2):196-209.
Stolt P, Bengtsson C, Nordmark B, Lindblad S, Lundberg I, Klareskog L, et al. Quantification of the influence of cigarette smoking on rheumatoid arthritis: results from a population based case-control study, using incident cases. Ann Rheum Dis. 2003; 62(9):835-41.
Uchtenhagen H, Rims C, Blahnik G, Chow I T, Kwok W W, Buckner J H, et al. Efficient ex vivo analysis of CD4+ T-cell responses using combinatorial HLA class II tetramer staining. Nature communications. 2016; 7:12614.
van Beers J J, Willemze A, Stammen-Vogelzangs J, Drijfhout J W, Toes R E, and Pruijn G J. Anti-citrullinated fibronectin antibodies in rheumatoid arthritis are associated with human leukocyte antigen-DRB1 shared epitope alleles. Arthritis Res Ther. 2012; 14(1):R35.
van Steendam K, Tilleman K, De Ceuleneer M, De Keyser F, Elewaut D, and Deforce D. Citrullinated vimentin as an important antigen in immune complexes from synovial fluid of rheumatoid arthritis patients with antibodies against citrullinated proteins. Arthritis Res Ther. 2010; 12(4):R132.
Wagner C A, Sokolove J, Lahey L J, Bengtsson C, Saevarsdottir S, Alfredsson L, et al. Identification of anticitrullinated protein antibody reactivities in a subset of anti-CCP− negative rheumatoid arthritis: association with cigarette smoking and HLA-DRB1 ‘shared epitope’ alleles. Ann Rheum Dis. 2015; 74(3):579-86.
Wambre E, DeLong J H, James E A, LaFond R E, Robinson D, and Kwok W W. Differentiation stage determines pathologic and protective allergen-specific CD4+ T-cell outcomes during specific immunotherapy. The Journal of allergy and clinical immunology. 2012; 129(2):544-51, 51.el-7.

1. An isolated citrullinated oligopeptide of no more than 14, 13, 12, 11, 10, or 9 amino acids, comprising a polypeptide of general formula (I) which comprises at least one arginine residue (R) that is citrullinated ([Cit]):
N—X—C (I)
said citrullinated oligopeptide being capable of eliciting an antigen-specific T cell response to human tenascin-C—, wherein [R/Cit] is either arginine or citrullinated arginine and wherein:
(a) X comprises an amino acid sequence that is selected from:

| | [Peptide 17A] |
|---|---|
| (SEQ ID NO: 1) | |
| | IS[R/Cit][R/Cit]GDMSS |
| | (TNC 874-882/Cit 876-877), |
| | [Peptide 17] |
| (SEQ ID NO: 2) | |
| | SLIS[R/Cit][R/Cit]GDMSSNPA |
| | (TNC 872-885/Cit 876-877), |
| | [Peptide 56] |
| (SEQ ID NO: 3) | |
| | GQYEL[R/Cit]VDL[R/Cit]DHGE |
| | (TNC 2068-2081/Cit 2073, 2077), |
| | and |
| | [Peptide 56B] |
| (SEQ ID NO: 4) | |
| | YEL[R/Cit]VDL[R/Cit]D |
| | (TNC 2070-2078/Cit 2073, 2077); |

(b) N is an amino terminus of the oligopeptide and consists of 0, 1, 2, 3, 4, or 5 amino acids that are independently selected from natural amino acids and non-natural amino acids; and
(c) C is a carboxy terminus of the oligopeptide and consists of 0, 1, 2, 3, 4, or 5 amino acids that are independently selected from natural amino acids and non-natural amino acids.

2. The isolated citrullinated oligopeptide of claim 1 in which the polypeptide of general formula (I) comprises at least two arginine residues that are citrullinated.

3. The isolated citrullinated oligopeptide of claim 2 wherein the amino acid sequence of X in general formula (I) comprises said at least two arginine residues that are citrullinated.

4. The isolated citrullinated oligopeptide of claim 1, wherein binding affinity of said oligopeptide of general formula (I) to a MHC class II molecule is greater than the binding affinity to the MHC class II molecule of a hypocitrullinated polypeptide comprising the oligopeptide of general formula (I) in which said at least one arginine residue is non-citrullinated.

5. The isolated citrullinated oligopeptide of claim 4, wherein said MHC class II molecule is an HLA-DRB1*04:01 molecule.

6. The isolated citrullinated oligopeptide of claim 3, wherein binding affinity of said oligopeptide of general formula (I) to a MHC class II molecule is greater than the binding affinity to the MHC class II molecule of a hypocitrullinated polypeptide comprising the oligopeptide of general formula (I) in which said at least two arginine residues in the amino acid sequence of X are non-citrullinated.

7. The isolated citrullinated oligopeptide of claim 6, wherein said MHC class II molecule is an HLA-DRB1*04:01 molecule.

8. The isolated citrullinated oligopeptide of claim 1 wherein the antigen-specific T cell response is a CD4+ T cell response.

9. The isolated citrullinated oligopeptide of claim 8 wherein the CD4+ T cell is a regulatory T (Treg) cell.

10. A composition, comprising the isolated citrullinated oligopeptide of claim 1 and a carrier.

11. (canceled)

12. The composition of claim 10 wherein the carrier is a solid carrier.

13. The composition of claim 12 wherein the solid carrier comprises a nanoparticle, a microparticle, a macroparticle, or a magnetic bead, or wherein the composition comprises an immunoassay substrate in which the solid carrier is a tube, an assay plate, a well of a multi-well plate, a membrane, a nanoparticle, a microparticle, a macroparticle, or a magnetic bead.

14. A method of detecting presence in a biological sample of an antibody that specifically binds to citrullinated human tenascin C, comprising:
(a) contacting (i) a biological sample obtained from a subject having or suspected of having one or more antibodies that specifically bind to human tenascin C, with (ii) an isolated citrullinated oligopeptide of no more than 14, 13, 12, 11, 10, or 9 amino acids, comprising a polypeptide of general formula (I) which comprises at least one arginine residue (R) that is citrullinated ([Cit]): N—X—C(I) wherein [R/Cit] is either arginine or citrullinated arginine,
under conditions and for a time sufficient for specific binding of said one or more antibodies to the citrullinated oligopeptide; and
(b) determining a test level of specific binding of said one or more antibodies to the citrullinated oligopeptide that is greater than a control level of specific binding of said one or more antibodies to a non-citrullinated polypeptide comprising the oligopeptide of general formula (I) in which there is no citrullinated arginine residue, and therefrom detecting presence in the biological sample of an antibody that specifically binds to citrullinated human tenascin C, wherein in general formula (I):
(1) X comprises an amino acid sequence that is selected from:

| | [Peptide 17A] |
|---|---|
| (SEQ ID NO: 1) | |
| | IS[R/Cit][R/Cit]GDMSS |
| | (TNC 874-882/Cit 876-877), |
| | [Peptide 17] |
| (SEQ ID NO: 2) | |
| | SLIS[R/Cit][R/Cit]GDMSSNPA |
| | (TNC 872-885/Cit 876-877), |
| | [Peptide 56] |
| (SEQ ID NO: 3) | |
| | GQYEL[R/Cit]VDL[R/Cit]DHGE |
| | (TNC 2068-2081/Cit 2073, 2077), |
| | and |
| | [Peptide 56B] |
| (SEQ ID NO: 4) | |
| | YEL[R/Cit]VDL[R/Cit]D |
| | (TNC 2070-2078/Cit 2073, 2077); |

(2) N is an amino terminus of the oligopeptide and consists of 0, 1, 2, 3, 4, or 5 amino acids that are independently selected from natural amino acids and non-natural amino acids; and
(3) C is a carboxy terminus of the oligopeptide and consists of 0, 1, 2, 3, 4, or 5 amino acids that are independently selected from natural amino acids and non-natural amino acids.

15.-21. (canceled)

22. An antigen-specific immunomodulatory composition comprising the isolated citrullinated oligopeptide of claim 1 and a pharmaceutically acceptable carrier, wherein the immunomodulatory composition is capable of inducing human tenascin C-specific immunological tolerance following administration to a human subject.

23. (canceled)

24. The immunomodulatory composition of claim 22 wherein the carrier is a solid carrier.

25. The immunomodulatory composition of claim 24 in which the solid carrier comprises a nanoparticle.

26. The composition of claim 22 which induces tenascin C-specific immunological tolerance that is MHC class II molecule-restricted, wherein the MHC class II molecule is HLA-DRB1*04:01.

27. A method of isolating one or a plurality of tenascin C-specific antibodies from a biological sample, comprising:
(a) incubating a reaction mixture that is formed by contacting (i) a biological sample obtained from a subject having or suspected of having one or more antibodies that specifically bind to human tenascin C, with (ii) an artificial antigen that comprises an isolated citrullinated oligopeptide of no more than 14, 13, 12, 11, 10, or 9 amino acids, comprising a polypeptide of general formula (I) which comprises at least one arginine residue (R) that is citrullinated ([Cit]): N—X—C(I) wherein [R/Cit] is either arginine or citrullinated arginine,
under conditions and for a time sufficient for specific binding of said one or more antibodies from the biological sample to the citrullinated oligopeptide to form one or more immune complexes comprising said antibodies specifically bound to the oligopeptide; and
(b) removing from the reaction mixture antibodies from the biological sample that are not specifically bound to the citrullinated oligopeptide, to recover said one or more immune complexes from the reaction mixture, and thereby isolating one or a plurality of tenascin-C-specific antibodies,
wherein in general formula (I):
(1) X comprises an amino acid sequence that is selected from:

| | [Peptide 17A |
|---|---|
| (SEQ ID NO: 1) | |
| | IS[R/Cit][R/Cit]GDMSS |
| | (TNC 874-882/Cit 876-877), |
| | [Peptide 17 |
| (SEQ ID NO: 2) | |
| | SLIS[R/Cit][R/Cit]GDMSSNPA |
| | (TNC 872-885/Cit 876-877), |
| | [Peptide 56 |
| (SEQ ID NO: 3) | |
| | GQYEL[R/Cit]VDL[R/Cit]DHGE |
| | (TNC 2068-2081/Cit 2073, 2077), |
| | and |
| | [Peptide 56B |
| (SEQ ID NO: 4) | |
| | YEL[R/Cit]VDL[R/Cit]D |
| | (TNC 2070-2078/Cit 2073, 2077); |

(2) N is an amino terminus of the oligopeptide and consists of 0, 1, 2, 3, 4, or 5 amino acids that are independently selected from natural amino acids and non-natural amino acids; and
(3) C is a carboxy terminus of the oligopeptide and consists of 0, 1, 2, 3, 4, or 5 amino acids that are independently selected from natural amino acids and non-natural amino acids.

28.-32. (canceled)

33. A method for in vitro preparation of antigen-pulsed antigen-presenting cells that are immunocompatible with a subject, comprising:
contacting in vitro, under conditions and for a time sufficient for antigen processing and presentation by antigen-presenting cells to take place, (i) a population of antigen-presenting cells that are immunocompatible with the subject, and (ii) the isolated citrullinated oligopeptide of claim 1, thereby obtaining antigen-pulsed antigen-presenting cells capable of eliciting an antigen-specific T cell response to human tenascin-C.

34.-37. (canceled)

38. An antigen-pulsed antigen-presenting cell comprising an antigen-presenting cell having an antigen-presenting cell surface on which is presented the citrullinated oligopeptide polypeptide of claim 1 capable of being specifically recognized by tenascin C-specific T cells.

39. A method of generating tenascin C-specific T cells, the method comprising contacting in vitro the antigen-pulsed antigen-presenting cell of claim 38 with one or a plurality of immunocompatible T-cells, under conditions and for a time sufficient to generate tenascin C-specific T-cells.

40.-46. (canceled)

47. A method for treating a condition characterized by a tenascin C-specific autoimmune response, comprising administering to a subject in need thereof a therapeutically effective amount of the engineered tenascin C-specific Treg cells generated according to the method of claim 39.

48.-50. (canceled)

51. An engineered tenascin C-specific Treg cell comprising at least one recombinant expression vector encoding a T-cell receptor polypeptide that specifically binds in a human class II HLA-restricted manner to a citrullinated oligopeptide of no more than 18, 17, 16, 15, 14, 13, 12, 11, 10, or 9 amino acids which comprises at least one arginine residue (R) that is citrullinated ([Cit]), said citrullinated oligopeptide comprising an amino acid sequence that is selected from:

| | [Peptide 17A] |
|---|---|
| (SEQ ID NO: 1) | |
| | IS[R/Cit][R/Cit]GDMSS |
| | (TNC 874-882/Cit 876-877), |
| | [Peptide 17] |
| (SEQ ID NO: 2) | |
| | SLIS[R/Cit][R/Cit]GDMSSNPA |
| | (TNC 872-885/Cit 876-877), |
| | [Peptide 56] |
| (SEQ ID NO: 3) | |
| | GQYEL[R/Cit]VDL[R/Cit]DHGE |
| | (TNC 2068-2081/Cit 2073, 2077), |
| | and |
| | [Peptide 56B] |
| (SEQ ID NO: 4) | |
| | YEL[R/Cit]VDL[R/Cit]D |
| | (TNC 2070-2078/Cit 2073, 2077). |

52. A method for treating a condition characterized by a tenascin C-specific autoimmune response in a subject, comprising adoptively transferring to the subject an effective amount of the engineered tenascin C-specific Treg cell of claim 51.

53. A method for detecting human tenascin C-specific T cell-mediated immune response activity in a subject, comprising:
(a) incubating in vitro (i) a biological sample obtained from the subject, said sample comprising T lymphocytes and antigen-presenting cells, with (ii) an isolated citrullinated oligopeptide of no more than 14, 13, 12, 11, 10, or 9 amino acids, comprising a polypeptide of general formula (I) which comprises at least one arginine residue (R) that is citrullinated ([Cit]):
N—X—C(I) wherein [R/Cit] is either arginine or citrullinated arginine,
under conditions and for a time sufficient for specific recognition by one or more T cell receptors (TCR) that are present in said T lymphocytes of the citrullinated oligopeptide to stimulate a T cell response activity; and
(b) determining a test level of the T cell response activity that is stimulated by specific TCR recognition of the citrullinated oligopeptide and that is greater than a control level of T cell response activity that is stimulated by incubating the biological sample with a non-citrullinated polypeptide comprising the oligopeptide of general formula (I) in which there is no citrullinated arginine residue, and therefrom detecting presence in the biological sample of human tenascin C-specific T cell-mediated immune response activity, wherein in general formula (I):
(a) X comprises an amino acid sequence that is selected from:

| | [Peptide 17A] |
|---|---|
| (SEQ ID NO: 1) | |
| | IS[R/Cit][R/Cit]GDMSS |
| | (TNC 874-882/Cit 876-877), |
| | [Peptide 17] |
| (SEQ ID NO: 2) | |
| | SLIS[R/Cit][R/Cit]GDMSSNPA |
| | (TNC 872-885/Cit 876-877), |
| | [Peptide 56] |
| (SEQ ID NO: 3) | |
| | GQYEL[R/Cit]VDL[R/Cit]DHGE |
| | (TNC 2068-2081/Cit 2073, 2077), |
| | and |
| | [Peptide56B] |
| (SEQ ID NO: 4) | |
| | YEL[R/Cit]VDL[R/Cit]D |
| | (TNC 2070-2078/Cit 2073, 2077); |

(b) N is an amino terminus of the oligopeptide and consists of 0, 1, 2, 3, 4, or 5 amino acids that are independently selected from natural amino acids and non-natural amino acids; and
(c) C is a carboxy terminus of the oligopeptide and consists of 0, 1, 2, 3, 4, or 5 amino acids that are independently selected from natural amino acids and non-natural amino acids.

54.-59. (canceled)

60. An isolated citrullinated oligopeptide of no more than 18, 17, 16, 15, 14, 13, 12, 11, 10, or 9 amino acids, comprising a polypeptide of general formula (I) which comprises at least one arginine residue (R) that is citrullinated ([Cit]):
N—X—C (I)
said citrullinated oligopeptide being capable of eliciting an antigen-specific T cell response to human tenascin-C, wherein [R/Cit] is either arginine or citrullinated arginine and wherein:
(a) X comprises an amino acid sequence that is selected from:

| | [Peptide 17A] |
|---|---|
| (SEQ ID NO: 1) | |
| | IS[R/Cit][R/Cit]GDMSS |
| | (TNC 874-882/Cit 876-877), |
| | [Peptide 17] |
| (SEQ ID NO: 2) | |
| | SLIS[R/Cit][R/Cit]GDMSSNPA |
| | (TNC 872-885/Cit 876-877), |
| | [Peptide 56] |
| (SEQ ID NO: 3) | |
| | GQYEL[R/Cit]VDL[R/Cit]DHGE |
| | TNC 2068-2081/Cit 2073, 2077), |
| | and |
| | [Peptide 56B] |
| (SEQ ID NO: 4) | |
| | YEL[R/Cit]VDL[R/Cit]D |
| | (TNC 2070-2078/Cit 2073, 2077); |

(b) N is an amino terminus of the oligopeptide and consists of 0, 1, 2, 3, 4, or 5 amino acids that are independently selected from natural amino acids and non-natural amino acids; and
(c) C is a carboxy terminus of the oligopeptide and consists of 0, 1, 2, 3, 4, or 5 amino acids that are independently selected from natural amino acids and non-natural amino acids.

61. An isolated polynucleotide comprising a nucleic acid sequence that encodes the polypeptide of general formula (I) of either claim 1 wherein [R/Cit] is arginine.

62.-65. (canceled)


SEQUENCE LISTING (78 sequences)

SEQ ID NO: 1 - protein, 9 aa, Artificial
  Synthetic oligopeptide
  MOD_RES (3)..(4): Xaa = Arg or citrullinated arginine
ISXXGDMSS

SEQ ID NO: 2 - protein, 14 aa, Artificial
  Synthetic oligopeptide
  MOD_RES (5)..(6): Xaa = Arg or citrullinated arginine
SLISXXGDMSSNPA

SEQ ID NO: 3 - protein, 14 aa, Artificial
  Synthetic oligopeptide
  MOD_RES (6)..(10): Xaa = Arg or citrullinated arginine
GQYELXVDLXDHGE

SEQ ID NO: 4 - protein, 9 aa, Artificial
  Synthetic oligopeptide
  MOD_RES (4)..(8): Xaa = Arg or citrullinated arginine
YELXVDLXD

SEQ ID NO: 5 - protein, 14 aa, Artificial
  Synthetic oligopeptide
  MOD_RES (1)..(5): Xaa = Arg or citrullinated arginine
XVDLXDHGETAFAV

SEQ ID NO: 6 - protein, 14 aa, Artificial
  Synthetic oligopeptide
  MOD_RES (10)..(14): Xaa = Arg or citrullinated arginine
ITAQGQYELXVDLX

SEQ ID NO: 7 - protein, 14 aa, Artificial
  Synthetic oligopeptide
  MOD_RES (5)..(9): Xaa = Arg or citrullinated arginine
QYELXVDLXDHGET

SEQ ID NO: 8 - protein, 14 aa, Artificial
  Synthetic oligopeptide
  MOD_RES (6)..(7): Xaa = Arg or citrullinated arginine
VSLISXXGDMSSNP

SEQ ID NO: 9 - protein, 14 aa, Artificial
  Synthetic oligopeptide
  MOD_RES (1)..(2): Xaa = Arg or citrullinated arginine
XXGDMSSNPAKETF

SEQ ID NO: 10 - protein, 14 aa, Artificial
  Synthetic oligopeptide
  MOD_RES (11)..(12): Xaa = Arg or citrullinated arginine
DTEYEVSLISXXGD

SEQ ID NO: 11 - protein, 14 aa, Artificial
  Synthetic oligopeptide
  MOD_RES (6)..(7): Xaa = Arg or citrullinated arginine
VSLISXXGDMSSNP

SEQ ID NO: 12 - protein, 18 aa, Artificial
  Synthetic oligopeptide
  MOD_RES 9,10: citrullinated arginine
EYEVSLISRRGDMSSNPA

SEQ ID NO: 13 - protein, 18 aa, Artificial
  Synthetic oligopeptide
  MOD_RES 6, 7: citrullinated arginine
VSLISRRGDMSSNPAKET

SEQ ID NO: 14 - protein, 18 aa, Artificial
  Synthetic oligopeptide
  MOD_RES 10, 14: citrullinated arginine
TAQQGQYELRVDLRDHGE

SEQ ID NO: 15 - protein, 18 aa, Artificial
  Synthetic oligopeptide
  MOD_RES 7,11: citrullinated arginine
QGQYELRVDLRDHGETAF

SEQ ID NO: 16 - protein, 13 aa, Artificial
  Synthetic oligopeptide
PKYVKQNTLKLAT

SEQ ID NO: 17 - protein, 15 aa, Artificial
  Synthetic oligopeptide
  MOD_RES 6,7: citrullinated arginine
VSLISRRGDMSSNPA

SEQ ID NO: 18 - protein, 15 aa, Artificial
  Synthetic oligopeptide
  MOD_RES 5: citrullinated arginine
PDGFRLSWTADEGVF

SEQ ID NO: 19 - protein, 15 aa, Artificial
  Synthetic oligopeptide
  MOD_RES 5: citrullinated arginine
VESFRITYVPITGGT

SEQ ID NO: 20 - protein, 15 aa, Artificial
  Synthetic oligopeptide
  MOD_RES 3, 5: citrullinated arginine
FDRYRLNYSLPTGQW

SEQ ID NO: 21 - protein, 15 aa, Artificial
  Synthetic oligopeptide
  MOD_RES 3: citrullinated arginine
LARWQPAIATVDSYV

SEQ ID NO: 22 - protein, 15 aa, Artificial
  Synthetic oligopeptide
  MOD_RES 5: citrullinated arginine
TKPLRAEIVTEAEPE

SEQ ID NO: 23 - protein, 15 aa, Artificial
  Synthetic oligopeptide
  MOD_RES 8: citrullinated arginine
SMAYHNGRSFSTFDK

SEQ ID NO: 24 - protein, 13 aa, Artificial
  Synthetic oligopeptide
PKYVKQNTLKLAT

SEQ ID NO: 25 - protein, 15 aa, Artificial
  Synthetic oligopeptide
  MOD_RES 6,7: citrullinated arginine
VSLISRRGDMSSNPA

SEQ ID NO: 26 - protein, 15 aa, Artificial
  Synthetic oligopeptide
  MOD_RES 7: citrullinated arginine
VSLISRRGDMSSNPA

SEQ ID NO: 27 - protein, 15 aa, Artificial
  Synthetic oligopeptide
  MOD_RES 6: citrullinated arginine
VSLISRRGDMSSNPA

SEQ ID NO: 28 - protein, 15 aa, Artificial
  Synthetic oligopeptide
VSLISRRGDMSSNPA

SEQ ID NO: 29 - protein, 15 aa, Artificial
  Synthetic oligopeptide
  MOD_RES 3,5: citrullinated arginine
FDRYRLNYSLPTGQW

SEQ ID NO: 30 - protein, 15 aa, Artificial
  Synthetic oligopeptide
  MOD_RES 5: citrullinated arginine
FDRYRLNYSLPTGQW

SEQ ID NO: 31 - protein, 15 aa, Artificial
  Synthetic oligopeptide
  MOD_RES 3: citrullinated arginine
FDRYRLNYSLPTGQW

SEQ ID NO: 32 - protein, 15 aa, Artificial
  Synthetic oligopeptide
FDRYRLNYSLPTGQW

SEQ ID NO: 33 - protein, 15 aa, Artificial
  Synthetic oligopeptide
  MOD_RES 5: citrullinated arginine
PDGFRLSWTADEGVF

SEQ ID NO: 34 - protein, 15 aa, Artificial
  Synthetic oligopeptide
PDGFRLSWTADEGVF

SEQ ID NO: 35 - protein, 15 aa, Artificial
  Synthetic oligopeptide
  MOD_RES 5: citrullinated arginine
VESFRITYVPITGGT

SEQ ID NO: 36 - protein, 15 aa, Artificial
  Synthetic oligopeptide
VESFRITYVPITGGT

SEQ ID NO: 37 - protein, 15 aa, Artificial
  Synthetic oligopeptide
  MOD_RES 7,11: citrullinated arginine
QGQYELRVDLRDHGE

SEQ ID NO: 38 - protein, 15 aa, Artificial
  Synthetic oligopeptide
  MOD_RES 11: citrullinated arginine
QGQYELRVDLRDHGE

SEQ ID NO: 39 - protein, 15 aa, Artificial
  Synthetic oligopeptide
  MOD_RES 7: citrullinated arginine
QGQYELRVDLRDHGE

SEQ ID NO: 40 - protein, 15 aa, Artificial
  Synthetic oligopeptide
QGQYELRVDLRDHGE

SEQ ID NO: 41 - protein, 15 aa, Artificial
  Synthetic oligopeptide
  MOD_RES 5: citrullinated arginine
IFDSRGNPTVEVDLF

SEQ ID NO: 42 - protein, 15 aa, Artificial
  Synthetic oligopeptide
  MOD_RES 7: citrullinated arginine
TSKGLFRAAVPSGAS

SEQ ID NO: 43 - protein, 15 aa, Artificial
  Synthetic oligopeptide
  MOD_RES 2: citrullinated arginine
KRIAKAVNEKSCNCL

SEQ ID NO: 44 - protein, 15 aa, Artificial
  Synthetic oligopeptide
TSKGLFRAAVPSGAS

SEQ ID NO: 45 - protein, 15 aa, Artificial
  Synthetic oligopeptide
KRIAKAVNEKSCNCL

SEQ ID NO: 46 - protein, 15 aa, Artificial
  Synthetic oligopeptide
  MOD_RES 9: citrullinated arginine
ATIKAEFVRAETPYM

SEQ ID NO: 47 - protein, 15 aa, Artificial
  Synthetic oligopeptide
  MOD_RES 7,10: citrullinated arginine
GKLYGIRDVRSTRDR

SEQ ID NO: 48 - protein, 12 aa, Artificial
  Synthetic oligopeptide
  MOD_RES 3,5: citrullinated arginine
GYRARPAKAAAT

SEQ ID NO: 49 - protein, 20 aa, Artificial
  Synthetic oligopeptide
  MOD_RES 6,11,13: citrullinated arginine
GVYATRSSAVRLRSSVPGVR

SEQ ID NO: 50 - protein, 14 aa, Artificial
  Synthetic oligopeptide
  MOD_RES 7: citrullinated arginine
FSSLNLRETNLDSL

SEQ ID NO: 51 - protein, 27 aa, Artificial
  Synthetic oligopeptide
  MOD_RES 13, 18, 23, 24: citrullinated arginine
CEHSIQFAEMKLRPSNFRNLEGRRKRC

SEQ ID NO: 52 - protein, 27 aa, Artificial
  Synthetic oligopeptide
CEHSIQFAEMKLRPSNFRNLEGRRKRC

SEQ ID NO: 53 - protein, 20 aa, Artificial
  Synthetic oligopeptide
  MOD_RES 10, 11: citrullinated arginine
CEYEVSLISRRGDMSSNPAC

SEQ ID NO: 54 - protein, 20 aa, Artificial
  Synthetic oligopeptide
CEYEVSLISRRGDMSSNPAC

SEQ ID NO: 55 - protein, 20 aa, Artificial
  Synthetic oligopeptide
  MOD_RES 9, 11: citrullinated arginine
CTPLAKFDRYRLNYSLPTGC

SEQ ID NO: 56 - protein, 20 aa, Artificial
  Synthetic oligopeptide
CTPLAKFDRYRLNYSLPTGC

SEQ ID NO: 57 - protein, 20 aa, Artificial
  Synthetic oligopeptide
  MOD_RES 11: citrullinated arginine
CVSDATPDGFRLSWTADEGC

SEQ ID NO: 58 - protein, 20 aa, Artificial
  Synthetic oligopeptide
CVSDATPDGFRLSWTADEGC

SEQ ID NO: 59 - protein, 20 aa, Artificial
  Synthetic oligopeptide
  MOD_RES 10: citrullinated arginine
CPTAQVESFRITYVPITGGC

SEQ ID NO: 60 - protein, 20 aa, Artificial
  Synthetic oligopeptide
CPTAQVESFRITYVPITGGC

SEQ ID NO: 61 - protein, 20 aa, Artificial
  Synthetic oligopeptide
  MOD_RES 8, 12: citrullinated arginine
CQGQYELRVDLRDHGETAFC

SEQ ID NO: 62 - protein, 20 aa, Artificial
  Synthetic oligopeptide
CQGQYELRVDLRDHGETAFC

SEQ ID NO: 63 - protein, 16 aa, Artificial
  Synthetic oligopeptide
  MOD_RES 8: citrullinated arginine
YEVSLISRRGDMSSNP

SEQ ID NO: 64 - protein, 16 aa, Artificial
  Synthetic oligopeptide
  MOD_RES 9: citrullinated arginine
YEVSLISRRGDMSSNP

SEQ ID NO: 65 - protein, 16 aa, Artificial
  Synthetic oligopeptide
  MOD_RES 5: citrullinated arginine
QYELRVDLRDHGETAF

SEQ ID NO: 66 - protein, 16 aa, Artificial
  Synthetic oligopeptide
  MOD_RES 9: citrullinated arginine
QYELRVDLRDHGETAF

SEQ ID NO: 67 - protein, 16 aa, Artificial
  Synthetic oligopeptide
  MOD_RES 9: citrullinated arginine
IQFAEMKLRPSNFRNL

SEQ ID NO: 68 - protein, 16 aa, Artificial
  Synthetic oligopeptide
  MOD_RES 9: Xaa = Cit
MKLRPSNFRNLEGRRK

SEQ ID NO: 69 - protein, 16 aa, Artificial
  Synthetic oligopeptide
  MOD_RES 12: citrullinated arginine
LRPSNFRNLEGRRKRA

SEQ ID NO: 70 - protein, 16 aa, Artificial
  Synthetic oligopeptide
  MOD_RES 13: citrullinated arginine
LRPSNFRNLEGRRKRA

SEQ ID NO: 71 - protein, 16 aa, Artificial
  Synthetic oligopeptide
  MOD_RES 15: citrullinated arginine
LRPSNFRNLEGRRKRA

SEQ ID NO: 72 - protein, 2201 aa, Artificial
  Synthetic oligopeptide
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGLDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDLTGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAQALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 73 - protein, 15 aa, Artificial
  Synthetic oligopeptide
VSLISRRGDMSSNPA

SEQ ID NO: 74 - protein, 15 aa, Artificial
  Synthetic oligopeptide
FDRYRLNYSLPTGQW

SEQ ID NO: 75 - protein, 15 aa, Artificial
  Synthetic oligopeptide
PDGFRLSWTADEGVF

SEQ ID NO: 76 - protein, 15 aa, Artificial
  Synthetic oligopeptide
VESFRITYVPITGGT

SEQ ID NO: 77 - protein, 15 aa, Artificial
  Synthetic oligopeptide
LARWQPAIATVDSYV

SEQ ID NO: 78 - protein, 15 aa, Artificial
  Synthetic oligopeptide
QGQYELRVDLRDHGE